(19) United States
(12) Patent Application Publication
(10) Pub. No.: US 2024/0307422 A1
(43) Pub. Date: Sep. 19, 2024

(54) BORONIC ACID DERIVATIVES AND THERAPEUTIC USES THEREOF

(71) Applicant: Qpex Biopharma, Inc., San Diego, CA (US)

(72) Inventors: Raja K. Reddy, San Diego, CA (US); Scott J. Hecker, Del Mar, CA (US)

(21) Appl. No.: 18/665,333

(22) Filed: May 15, 2024

Related U.S. Application Data
(62) Division of application No. 17/048,052, filed on Oct. 15, 2020, now Pat. No. 12,016,868.
(60) Provisional application No. 62/660,729, filed on Apr. 20, 2018.

(51) Int. Cl. A61K 31/69; A61K 31/407; A61K 31/4196; A61K 31/427; A61K 31/43; A61K 31/431; A61K 31/4439; A61K 31/545; A61K 31/546; A61P 31/04; C07F 5/02; C07F 5/04

(52) U.S. Cl. CPC A61K 31/69; A61K 31/407; A61K 31/4196; A61K 31/427; A61K 31/43; A61K 31/431; A61K 31/4439; A61K 31/545; A61K 31/546; A61P 31/04; C07F 5/025; C07F 5/04

(57) ABSTRACT

Disclosed herein are antimicrobial compounds, compositions, pharmaceutical compositions, and the use and preparation thereof. Some embodiments relate to boronic acid derivatives and their use as therapeutic agents.

### CROSS REFERENCE TO RELATED APPLICATIONS

The present application is a divisional of U.S. patent application Ser. No. 17/048,052, filed Oct. 15, 2020, which is a U.S. National Phase of International Application No. PCT/US2019/027844, filed on Apr. 17, 2019, and published on Oct. 24, 2019, as WO 2019/204419, which claims the benefit of U.S. Provisional Application No. 62/660,729, filed Apr. 20, 2018, entitled “BORONIC ACID DERIVATIVES AND THERAPEUTIC USES THEREOF”, which is incorporated by reference in its entirety.

### BACKGROUND

### Field of the Invention

The present invention relates to the fields of chemistry and medicine. More particularly, the present invention relates to boronic acid antimicrobial compounds, compositions, their preparation, and their use as therapeutic agents.

### Description of the Related Art

Antibiotics have been effective tools in the treatment of infectious diseases during the last half-century. From the development of antibiotic therapy to the late 1980s there was almost complete control over bacterial infections in developed countries. However, in response to the pressure of antibiotic usage, multiple resistance mechanisms have become widespread and are threatening the clinical utility of anti-bacterial therapy. The increase in antibiotic resistant strains has been particularly common in major hospitals and care centers. The consequences of the increase in resistant strains include higher morbidity and mortality, longer patient hospitalization, and an increase in treatment costs.

Various bacteria have evolved β-lactam deactivating enzymes, namely, β-lactamases, that counter the efficacy of the various β-lactam antibiotics. β-lactamases can be grouped into 4 classes based on their amino acid sequences, namely, Ambler classes A, B, C, and D. Enzymes in classes A, C, and D include active-site serine β-lactamases, and class B enzymes, which are encountered less frequently, are Zn-dependent. These enzymes catalyze the chemical degradation of β-lactam antibiotics, rendering them inactive. Some β-lactamases can be transferred within and between various bacterial strains and species. The rapid spread of bacterial resistance and the evolution of multi-resistant strains severely limits β-lactam treatment options available.

The increase of class D β-lactamase-expressing bacterium strains such as Acinetobacter baumannii has become an emerging multidrug-resistant threat. A. baumannii strains express A, C, and D class β-lactamases. The class D β-lactamases such as the OXA families are particularly effective at destroying carbapenem type β-lactam antibiotics, e.g., imipenem, the active carbapenems component of Merck's Primaxin® (Montefour, K.; et al. Crit. Care Nurse 2008, 28, 15; Perez, F. et al. Expert Rev. Anti Infect. Ther. 2008, 6, 269; Bou, G.; Martinez-Beltran, J. Antimicrob. Agents Chemother. 2000, 40, 428. 2006, 50, 2280; Bou, G. et al, J. Antimicrob. Agents Chemother. 2000, 44, 1556). This has imposed a pressing threat to the effective use of drugs in that category to treat and prevent bacterial infections. Indeed the number of catalogued serine-based β-lactamases has exploded from less than ten in the 1970s to over 300 variants. These issues fostered the development of five “generations” of cephalosporins. When initially released into clinical practice, extended-spectrum cephalosporins resisted hydrolysis by the prevalent class A β-lactamases, TEM-1 and SHV-1. However, the development of resistant strains by the evolution of single amino acid substitutions in TEM-1 and SHV-1 resulted in the emergence of the extended-spectrum β-lactamase (ESBL) phenotype.

New β-lactamases have recently evolved that hydrolyze the carbapenem class of antimicrobials, including imipenem, biapenem, doripenem, meropenem, and ertapenem, as well as other β-lactam antibiotics. These carbapenemases belong to molecular classes A, B, and D. Class A carbapenemases of the KPC-type predominantly in Klebsiella pneumoniae but now also reported in other Enterobacteriaceae, Pseudomonas aeruginosa and Acinetobacter baumannii. The KPC carbapenemase was first described in 1996 in North Carolina, but since then has disseminated widely in the US. It has been particularly problematic in the New York City area, where several reports of spread within major hospitals and patient morbidity have been reported. These enzymes have also been recently reported in France, Greece, Sweden, United Kingdom, and an outbreak in Germany has recently been reported. Treatment of resistant strains with carbapenems can be associated with poor outcomes.

The zinc-dependent class B metallo-β-lactamases are represented mainly by the VIM, IMP, and NDM types. IMP and VIM-producing K. pneumonia were first observed in 1990s in Japan and 2001 in Southern Europe, respectively. IMP-positive strains remain frequent in Japan and have also caused hospital outbreaks in China and Australia. However, dissemination of IMP-producing Enterobacteriaceae in the rest of the word appears to be somewhat limited. VIM-producing enterobacteria can be frequently isolated in Mediterranean countries, reaching epidemic proportions in Greece. Isolation of VIM-producing strains remains low in Northern Europe and in the United States. In stark contrast, a characteristic of NDM-producing K. pneumonia isolates has been their rapid dissemination from their epicenter, the Indian subcontinent, to Western Europe, North America, Australia and Far East. Moreover, NDM genes have spread rapidly to various species other than K. pneumonia.

The plasmid-expressed class D carbapenemases belong to OXA-48 type. OXA-48 producing K. pneumonia was first detected in Turkey, in 2001. The Middle East and North Africa remain the main centers of infection. However, recent isolation of OXA-48-type producing organisms in India, Senegal and Argentina suggest the possibility of a global expansion. Isolation of OXA-48 in bacteria other than K. pneumonia underlines the spreading potential of OXA-48.

Treatment of strains producing any of these carbapenemases with carbapenems can be associated with poor outcomes.

Another mechanism of β-lactamase mediated resistance to carbapenems involves permeability or efflux mechanisms combined with hyper production of β-lactamases. One example is the loss of a porin combined with hyperproduction of ampC β-lactamase resulting in resistance to imipenem in Pseudomonas aeruginosa. Efflux pump overexpression combined with hyperproduction of the ampC β-lactamase can also result in resistance to a carbapenem such as meropenem.

Thus, there is a need for improved β-lactamase inhibitors.

### SUMMARY OF THE INVENTION

Some embodiments disclosed herein relate to a compound having the structure of the Formula (I) or Formula (II):

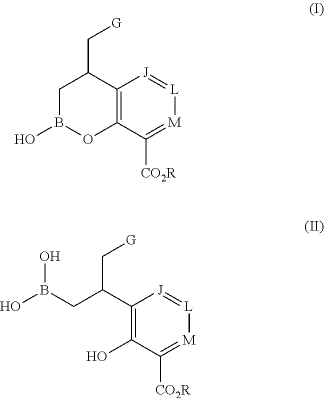

or a pharmaceutically acceptable salt thereof, wherein:

G is selected from the group consisting of —OR¹—C(O)R¹, —C(O)(CH₂)₀₋₃SR¹, —C(O)(CH₂)₁₋₃R¹, —C(O)OR₁, —C(O)NR¹R², —C(O)NR¹OR², —N₃, —NR¹R², —NR¹C(O)R², —NR¹C(O)NR²R³, —NR¹C(O)OR², —NR¹S(O)₂R², —NR¹S(O)₂NR²R³, —C(═NR¹)R², —C(═NR¹)NR²R³, —NR¹CR²(═NR³), —NR¹C(═NR²)NR³R⁴, —S(O)₂R¹, optionally substituted C₁₋₁₀ alkyl, optionally substituted C₂₋₁₀ alkenyl, optionally substituted C₂₋₁₀ alkynyl, optionally substituted C₃₋₇ carbocyclyl, optionally substituted 5-10 membered heterocyclyl, optionally substituted C₆₋₁₀ aryl, optionally substituted 5-10 membered heteroaryl, optionally substituted C₃₋₇ carbocyclyl-C₁₋₆ alkyl, optionally substituted 5-10 membered heterocyclyl-C₁₋₆ alkyl, optionally substituted C₆₋₁₀ aryl-C₁₋₆ alkyl, and optionally substituted 5-10 membered heteroaryl-C₁₋₆ alkyl;

R¹, R², R³, and R⁴ are each independently selected from the group consisting of —H, optionally substituted C₁₋₄ alkyl, optionally substituted C₃₋₇ carbocyclyl, optionally substituted 4-10 membered heterocyclyl, optionally substituted C₆₋₁₀ aryl, optionally substituted C₆₋₁₀ aryl-C₁₋₆ alkyl, and optionally substituted 5-10 membered heteroaryl;

J is selected from the group consisting of CR⁵ and N;

L is selected from the group consisting of CR⁶ and N;

M is selected from the group consisting of CR⁷ and N;

R⁵, R⁶, and R⁷ are each independently selected from the group consisting of —H, —OR⁸, halogen, —CF₃, optionally substituted C₂-C₆ alkenyl, optionally substituted C₂-C₆ alkynyl, optionally substituted C₁-C₆ heteroalkyl, optionally substituted C₃-C₇ carbocyclyl, optionally substituted 5-10 membered heterocyclyl, optionally substituted C₆₋₁₀ aryl, optionally substituted 5-10 membered heteroaryl, cyano, C₁-C₆ alkoxy(C₁-C₆)alkyl, aryloxy, and sulfhydryl (mercapto);

R⁸ is selected from the group consisting of hydrogen, optionally substituted C₁₋₄ alkyl, optionally substituted C₃₋₇ carbocyclyl, optionally substituted 4-10 membered heterocyclyl, optionally substituted C₆₋₁₀ aryl, and optionally substituted 5-10 membered heteroaryl;

R is selected from the group consisting of —H, —C₁₋₉ alkyl, —CR⁹R¹⁰OC(O)C₁₋₉alkyl, —CR⁹R¹⁰OC(O)OC₁₋₉alkyl, —CR⁹R¹⁰OC(O)C₆₋₁₀aryl, —CR⁹R¹⁰OC(O)OC₆₋₁₀aryl,

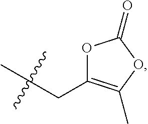 [SMILES: *Cc1oc(=O)oc1C]

—CR⁹R¹⁰OC(O)C₃₋₇carbocyclyl, —CR⁹R¹⁰OC(O)OC₃₋₇carbocyclyl, —CR⁹R¹⁰OC(O)(5-10 membered heterocyclyl), and —CR⁹R¹⁰OC(O)O(5-10 membered heterocyclyl); and

R⁹ and R¹⁰ are independently selected from the group consisting of —H, optionally substituted C₁₋₄ alkyl, optionally substituted C₃₋₇ carbocyclyl, optionally substituted 5-10 membered heterocyclyl, optionally substituted C₆₋₁₀ aryl, and optionally substituted 5-10 membered heteroaryl.

Other embodiments disclosed herein include a pharmaceutical composition comprising a therapeutically effective amount of a compound disclosed herein and a pharmaceutically acceptable excipient.

Other embodiments disclosed herein include a method of treating or preventing a bacterial infection, comprising administering to a subject in need thereof a compound disclosed herein.

### DETAILED DESCRIPTION

In some embodiments, compounds that contain a boronic acid moiety are provided that act as antimicrobial agents and/or as potentiators of antimicrobial agents. Various embodiments of these compounds include compounds having the structures of Formula (I) as described above or pharmaceutically acceptable salts thereof.

Some embodiments of compounds of Formulas (I) and (II) or their pharmaceutically acceptable salts have the following stereochemistry as shown in the structure of Formula (Ia) or Formula (IIa):

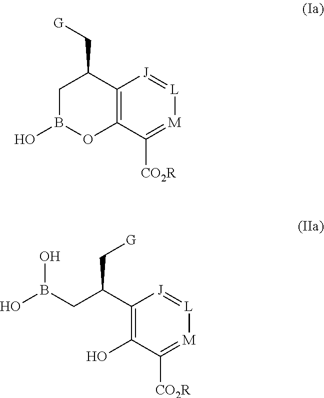

Some embodiments of compounds of Formulas (I) and (II) or their pharmaceutically acceptable salts have the following stereochemistry as shown in the structure of Formula (Ib) or Formula (IIb):

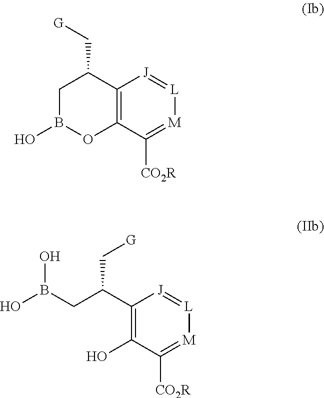

Some embodiments of compounds of Formulas (I) and (II) or their pharmaceutically acceptable salts include compounds having the structure of Formula (Ic) or Formula (IIc):

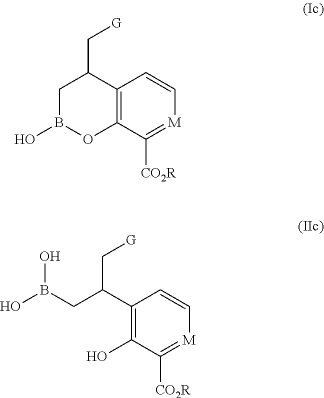

In some embodiments of compounds of Formula (I), (Ia), (Ib), (Ic), (II), (IIa), (IIb), or (IIc) G is selected from the group consisting of —OR^{1,} N₃, —NR¹R², NR¹C(O)R², optionally substituted C₁₋₄ alkyl, and optionally substituted 5-10 membered heteroaryl;

In some embodiments of compounds of Formula (I), (Ia), (Ib), (Ic), (II), (IIa), (IIb), or (IIc) G is —OR¹.

In some embodiments of compounds of Formula (I), (Ia), (Ib), (Ic), (II), (IIa), (IIb), or (IIc) G is selected from the group consisting of —OH, —OMe, —OBn, —CH₂OH, N₃, NH₂, —NHC(═O)H, —NHC(═O)CH₃, and

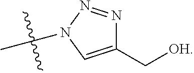 [SMILES: *n1cc(CO)nn1]

In some embodiments of compounds of Formula (I), (Ia), (Ib), (Ic), (II), (IIa), (IIb), or (IIc) G is selected from the group consisting of —OH and —OBn.

In some embodiments of compounds of Formula (I), (Ia), (Ib), (Ic), (II), (IIa), (IIb), or (IIc) G is —OH.

In some embodiments of compounds of Formula (I), (Ia), (Ib), (Ic), (II), (IIa), (IIb), or (IIc) M is CR⁷ and R⁷ is selected from the group consisting of —H, —OR⁸, and halogen.

In some embodiments of compounds of Formula (I), (Ia), (Ib), (Ic), (II), (IIa), (IIb), or (IIc) R⁸ is optionally substituted C₁₋₄ alkyl.

In some embodiments of compounds of Formula (I), (Ia), (Ib), (Ic), (II), (IIa), (IIb), or (IIc) M is selected from the group consisting of —CH, —COMe, CF, and N.

In some embodiments of compounds of Formula (I), (Ia), (Ib), (Ic), (II), (IIa), (IIb), or (IIc) M is —COMe.

In some embodiments of compounds of Formula (I), (Ia), (Ib), (Ic), (II), (IIa), (IIb), or (IIc) R is —H.

Some embodiments include a compound selected from the group consisting of:

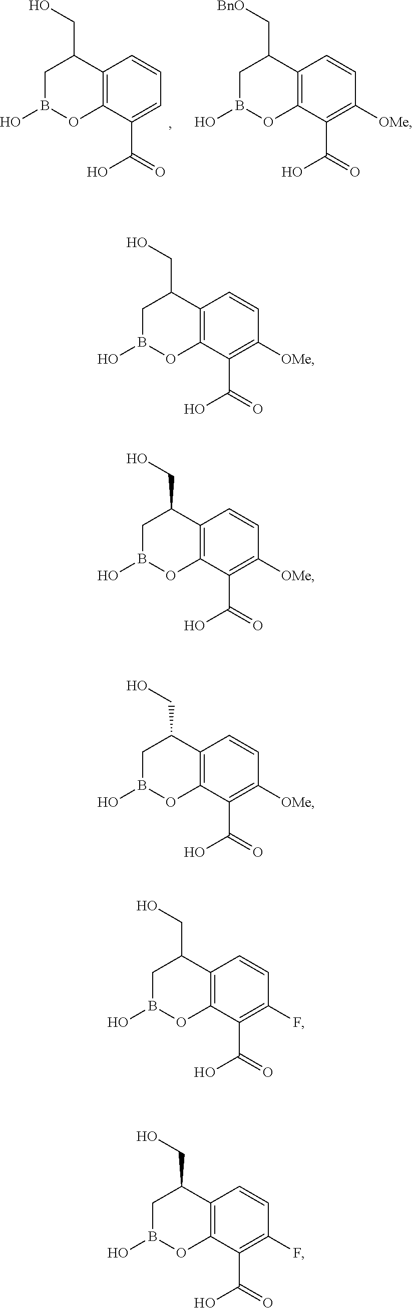

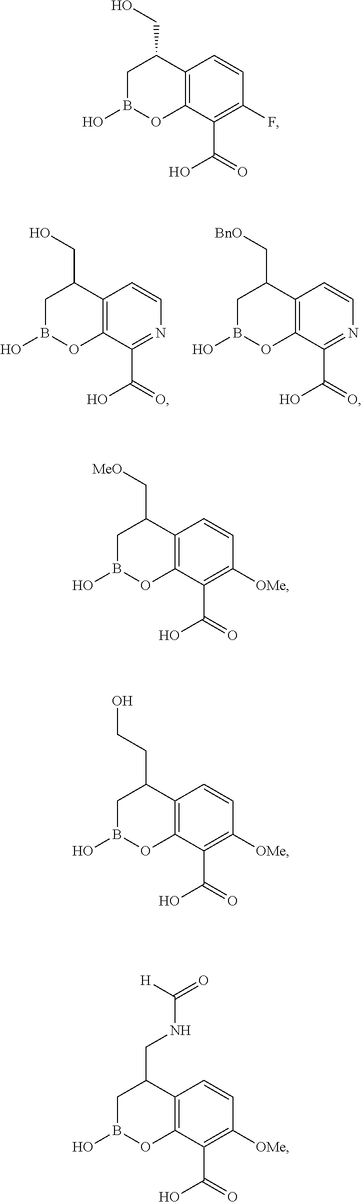 [SMILES: O=C(O)c1c(F)ccc2c1OB(O)C[C@@H]2CO ; O=C(O)c1nccc2c1OB(O)CC2CO ; O=C(O)c1nccc2c1OB(O)CC2COCc1ccccc1 ; COCC1CB(O)Oc2c1ccc(OC)c2C(=O)O ; COc1ccc2c(c1C(=O)O)OB(O)CC2CCO ; COc1ccc2c(c1C(=O)O)OB(O)CC2CNC=O]

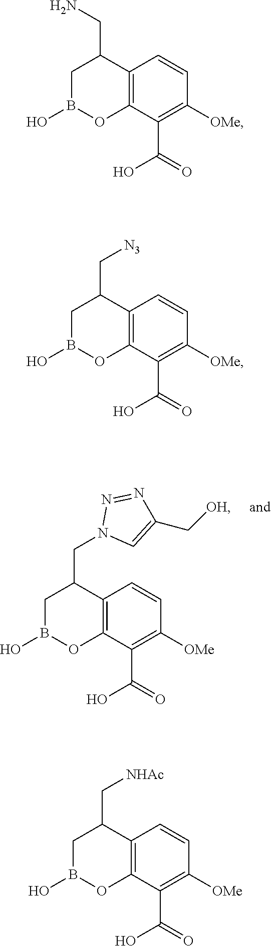 [SMILES: COc1ccc2c(c1C(=O)O)OB(O)CC2CN ; COc1ccc2c(c1C(=O)O)OB(O)CC2CN=[N+]=[N-] ; COc1ccc2c(c1C(=O)O)OB(O)CC2Cn1cc(CO)nn1 ; "and" ; COc1ccc2c(c1C(=O)O)OB(O)CC2CNC(C)=O]

or a pharmaceutically acceptable salt thereof.

Where the compounds disclosed herein have at least one chiral center, they may exist as individual enantiomers and diastereomers or as mixtures of such isomers, including racemates. Separation of the individual isomers or selective synthesis of the individual isomers is accomplished by application of various methods which are well known to practitioners in the art. Unless otherwise indicated, all such isomers and mixtures thereof are included in the scope of the compounds disclosed herein. Furthermore, compounds disclosed herein may exist in one or more crystalline or amorphous forms. Unless otherwise indicated, all such forms are included in the scope of the compounds disclosed herein including any polymorphic forms. In addition, some of the compounds disclosed herein may form solvates with water (i.e., hydrates) or common organic solvents. Unless otherwise indicated, such solvates are included in the scope of the compounds disclosed herein.

The skilled artisan will recognize that some structures described herein may be resonance forms or tautomers of compounds that may be fairly represented by other chemical structures, even when kinetically; the artisan recognizes that such structures may only represent a very small portion of a sample of such compound(s). Such compounds are considered within the scope of the structures depicted, though such resonance forms or tautomers are not represented herein.

In some embodiments, due to the facile exchange of boron esters, the compounds described herein may convert to or exist in equilibrium with alternate forms. Accordingly, in some embodiments, the compounds described herein may exist in combination with one or more of these forms. For example, as shown below, the compounds disclosed herein may exist in cyclic boronate monoesters as formula I or in acyclic form as boronic acids as formula II, or may exist as a mixture of the two forms depending on the medium. When G is —OH or NHR₂, compounds of formula II may also cyclize to give compounds of formula III where G″ is O or NR₂. In this case, the compounds may exist as a mixture of all three forms depending on the medium.

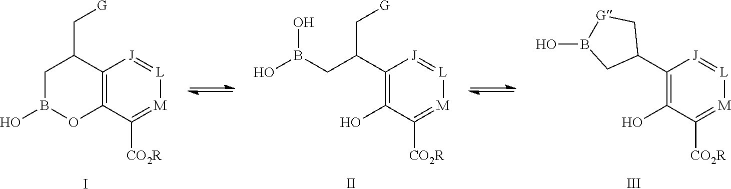

In some embodiments, the compounds described herein may exist in cyclic dimeric form as Formula (A) or trimeric form as Formula (B), tetrameric form as Formula (C) as shown below, or acylic dimeric, trimeric or tetrameric forms and the like.

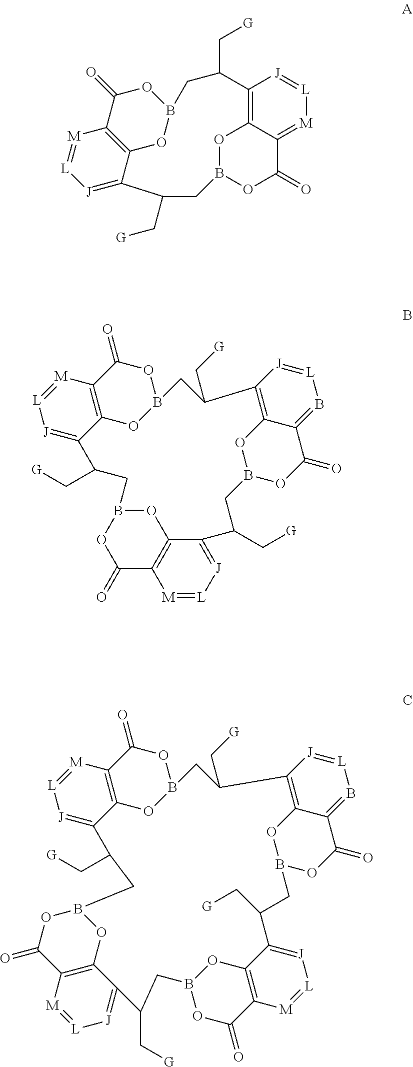

Isotopes may be present in the compounds described. Each chemical element as represented in a compound structure may include any isotope of said element. For example, in a compound structure a hydrogen atom may be explicitly disclosed or understood to be present in the compound. At any position of the compound that a hydrogen atom may be present, the hydrogen atom can be any isotope of hydrogen, including but not limited to hydrogen-1 (protium) and hydrogen-2 (deuterium). Thus, reference herein to a compound encompasses all potential isotopic forms unless the context clearly dictates otherwise.

### Definitions

Unless defined otherwise, all technical and scientific terms used herein have the same meaning as is commonly understood by one of ordinary skill in the art to which this disclosure belongs. All patents, applications, published applications, and other publications referred to herein are incorporated by reference in their entirety. In the event that there is a plurality of definitions for a term herein, those in this section prevail unless stated otherwise.

A “prodrug” refers to an agent that is converted into the parent drug in vivo. Prodrugs are often useful because, in some situations, they may be easier to administer than the parent drug. They may, for instance, be bioavailable by oral administration whereas the parent is not. The prodrug may also have improved solubility in pharmaceutical compositions over the parent drug. An example, without limitation, of a prodrug would be a compound which is administered as an ester (the “prodrug”) to facilitate transmittal across a cell membrane where water solubility is detrimental to mobility but which then is metabolically hydrolyzed to the carboxylic acid, the active entity, once inside the cell where water-solubility is beneficial. A further example of a prodrug might be a short peptide (polyaminoacid) bonded to an acid group where the peptide is metabolized to reveal the active moiety. Conventional procedures for the selection and preparation of suitable prodrug derivatives are described, for example, in Design of Prodrugs, (ed. H. Bundgaard, Elsevier, 1985), which is hereby incorporated herein by reference in its entirety.

The term “pro-drug ester” refers to derivatives of the compounds disclosed herein formed by the addition of any of several ester-forming groups that are hydrolyzed under physiological conditions. Examples of pro-drug ester groups include pivoyloxymethyl, acetoxymethyl, phthalidyl, indanyl and methoxymethyl, as well as other such groups known in the art, including a (5-R-2-oxo-1,3-dioxolen-4-yl)methyl group. Other examples of pro-drug ester groups can be found in, for example, T. Higuchi and V. Stella, in “Pro-drugs as Novel Delivery Systems”, Vol. 14, A.C.S. Symposium Series, American Chemical Society (1975); and “Bioreversible Carriers in Drug Design: Theory and Application”, edited by E. B. Roche, Pergamon Press: New York, 14-21 (1987) (providing examples of esters useful as prodrugs for compounds containing carboxyl groups). Each of the above-mentioned references is herein incorporated by reference in their entirety.

“Metabolites” of the compounds disclosed herein include active species that are produced upon introduction of the compounds into the biological milieu.

“Solvate” refers to the compound formed by the interaction of a solvent and a compound described herein, a metabolite, or salt thereof. Suitable solvates are pharmaceutically acceptable solvates including hydrates.

The term “pharmaceutically acceptable salt” refers to salts that retain the biological effectiveness and properties of a compound, which are not biologically or otherwise undesirable for use in a pharmaceutical. In many cases, the compounds herein are capable of forming acid and/or base salts by virtue of the presence of amino and/or carboxyl groups or groups similar thereto. Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, aluminum, and the like; particularly preferred are the ammonium, potassium, sodium, calcium and magnesium salts. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine. Many such salts are known in the art, as described in WO 87/05297, Johnston et al., published Sep. 11, 1987 (incorporated by reference herein in its entirety). Some examples of pharmaceutically acceptable base addition salts of the compounds disclosed herein have the structure of Formula (I-salt) or (II-salt):

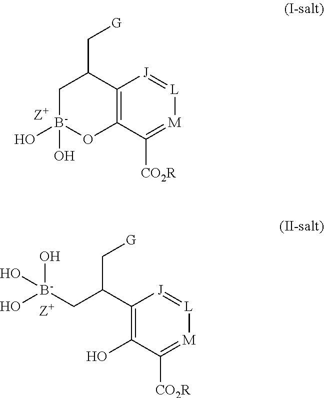

wherein Z can be an alkali metal or NH₄⁺.

As used herein, “Cₐ to C_{b}” or “C_{a-b}” in which “a” and “b” are integers refer to the number of carbon atoms in the specified group. That is, the group can contain from “a” to “b”, inclusive, carbon atoms. Thus, for example, a “C₁ to C₄ alkyl” or “C₁₋₄ alkyl” group refers to all alkyl groups having from 1 to 4 carbons, that is, CH₃—, CH₃CH₂—, CH₃CH₂CH₂—, (CH₃)₂CH—, CH₃CH₂CH₂CH₂—, CH₃CH₂CH(CH₃)— and (CH₃)₃C—.

The term “halogen” or “halo,” as used herein, means any one of the radio-stable atoms of column 7 of the Periodic Table of the Elements, e.g., fluorine, chlorine, bromine, or iodine, with fluorine and chlorine being preferred.

As used herein, “alkyl” refers to a straight or branched hydrocarbon chain that is fully saturated (i.e., contains no double or triple bonds). The alkyl group may have 1 to 20 carbon atoms (whenever it appears herein, a numerical range such as “1 to 20” refers to each integer in the given range; e.g., “1 to 20 carbon atoms” means that the alkyl group may consist of 1 carbon atom, 2 carbon atoms, 3 carbon atoms, etc., up to and including 20 carbon atoms, although the present definition also covers the occurrence of the term “alkyl” where no numerical range is designated). The alkyl group may also be a medium size alkyl having 1 to 9 carbon atoms. The alkyl group could also be a lower alkyl having 1 to 4 carbon atoms. The alkyl group of the compounds may be designated as “C₁₋₄ alkyl” or similar designations. By way of example only, “C₁₋₄ alkyl” indicates that there are one to four carbon atoms in the alkyl chain, i.e., the alkyl chain is selected from the group consisting of methyl, ethyl, propyl, iso-propyl, n-butyl, iso-butyl, sec-butyl, and t-butyl. Typical alkyl groups include, but are in no way limited to, methyl, ethyl, propyl, isopropyl, butyl, isobutyl, tertiary butyl, pentyl, hexyl, and the like.

As used herein, “alkoxy” refers to the formula-OR wherein R is an alkyl as is defined above, such as “C₁₋₉ alkoxy”, including but not limited to methoxy, ethoxy, n-propoxy, 1-methylethoxy (isopropoxy), n-butoxy, iso-butoxy, sec-butoxy, tert-butoxy, and the like.

As used herein, “alkylthio” refers to the formula-SR wherein R is an alkyl as is defined above, such as “C₁₋₉ alkylthio” and the like, including but not limited to methylmercapto, ethylmercapto, n-propylmercapto, 1-methylethylmercapto (isopropylmercapto), n-butylmercapto, iso-butylmercapto, sec-butylmercapto, tert-butylmercapto, and the like.

As used herein, “alkenyl” refers to a straight or branched hydrocarbon chain containing one or more double bonds. The alkenyl group may have 2 to 20 carbon atoms, although the present definition also covers the occurrence of the term “alkenyl” where no numerical range is designated. The alkenyl group may also be a medium size alkenyl having 2 to 9 carbon atoms. The alkenyl group could also be a lower alkenyl having 2 to 4 carbon atoms. The alkenyl group of the compounds may be designated as “C₂₋₄ alkenyl” or similar designations. By way of example only, “C₂₋₄ alkenyl” indicates that there are two to four carbon atoms in the alkenyl chain, i.e., the alkenyl chain is selected from the group consisting of ethenyl, propen-1-yl, propen-2-yl, propen-3-yl, buten-1-yl, buten-2-yl, buten-3-yl, buten-4-yl, 1-methyl-propen-1-yl, 2-methyl-propen-1-yl, 1-ethyl-ethen-1-yl, 2-methyl-propen-3-yl, buta-1,3-dienyl, buta-1,2,-dienyl, and buta-1,2-dien-4-yl. Typical alkenyl groups include, but are in no way limited to, ethenyl, propenyl, butenyl, pentenyl, hexenyl, and the like.

As used herein, “alkynyl” refers to a straight or branched hydrocarbon chain containing one or more triple bonds. The alkynyl group may have 2 to 20 carbon atoms, although the present definition also covers the occurrence of the term “alkynyl” where no numerical range is designated. The alkynyl group may also be a medium size alkynyl having 2 to 9 carbon atoms. The alkynyl group could also be a lower alkynyl having 2 to 4 carbon atoms. The alkynyl group of the compounds may be designated as “C₂₋₄ alkynyl” or similar designations. By way of example only, “C₂₋₄ alkynyl” indicates that there are two to four carbon atoms in the alkynyl chain, i.e., the alkynyl chain is selected from the group consisting of ethynyl, propyn-1-yl, propyn-2-yl, butyn-1-yl, butyn-3-yl, butyn-4-yl, and 2-butynyl. Typical alkynyl groups include, but are in no way limited to, ethynyl, propynyl, butynyl, pentynyl, hexynyl, and the like.

As used herein, “heteroalkyl” refers to a straight or branched hydrocarbon chain containing one or more heteroatoms, that is, an element other than carbon, including but not limited to, nitrogen, oxygen and sulfur, in the chain backbone. The heteroalkyl group may have 1 to 20 carbon atoms although the present definition also covers the occurrence of the term “heteroalkyl” where no numerical range is designated. The heteroalkyl group may also be a medium size heteroalkyl having 1 to 9 carbon atoms. The heteroalkyl group could also be a lower heteroalkyl having 1 to 4 carbon atoms. The heteroalkyl group of the compounds may be designated as “C₁₋₄ heteroalkyl” or similar designations. The heteroalkyl group may contain one or more heteroatoms. By way of example only, “C₁₋₄ heteroalkyl” indicates that there are one to four carbon atoms in the heteroalkyl chain and additionally one or more heteroatoms in the backbone of the chain.

The term “aromatic” refers to a ring or ring system having a conjugated pi electron system and includes both carbocyclic aromatic (e.g., phenyl) and heterocyclic aromatic groups (e.g., pyridine). The term includes monocyclic or fused-ring polycyclic (i.e., rings which share adjacent pairs of atoms) groups provided that the entire ring system is aromatic.

As used herein, “aryl” refers to an aromatic ring or ring system (i.e., two or more fused rings that share two adjacent carbon atoms) containing only carbon in the ring backbone. When the aryl is a ring system, every ring in the system is aromatic. The aryl group may have 6 to 18 carbon atoms, although the present definition also covers the occurrence of the term “aryl” where no numerical range is designated. In some embodiments, the aryl group has 6 to 10 carbon atoms. The aryl group may be designated as “C₆₋₁₀ aryl,” “C₆ or C₁₀ aryl,” or similar designations. Examples of aryl groups include, but are not limited to, phenyl, naphthyl, azulenyl, and anthracenyl.

As used herein, “aryloxy” and “arylthio” refers to RO— and RS—, in which R is an aryl as is defined above, such as “C₆₋₁₀ aryloxy” or “C₆₋₁₀ arylthio” and the like, including but not limited to phenyloxy.

An “aralkyl” or “arylalkyl” is an aryl group connected, as a substituent, via an alkylene group, such “C₇₋₁₄ aralkyl” and the like, including but not limited to benzyl, 2-phenylethyl, 3-phenylpropyl, and naphthylalkyl. In some cases, the alkylene group is a lower alkylene group (i.e., a C₁₋₄ alkylene group).

As used herein, “heteroaryl” refers to an aromatic ring or ring system (i.e., two or more fused rings that share two adjacent atoms) that contain(s) one or more heteroatoms, that is, an element other than carbon, including but not limited to, nitrogen, oxygen and sulfur, in the ring backbone. When the heteroaryl is a ring system, every ring in the system is aromatic. The heteroaryl group may have 5-18 ring members (i.e., the number of atoms making up the ring backbone, including carbon atoms and heteroatoms), although the present definition also covers the occurrence of the term “heteroaryl” where no numerical range is designated. In some embodiments, the heteroaryl group has 5 to 10 ring members or 5 to 7 ring members. The heteroaryl group may be designated as “5-7 membered heteroaryl,” “5-10 membered heteroaryl,” or similar designations. Examples of heteroaryl rings include, but are not limited to, furyl, thienyl, phthalazinyl, pyrrolyl, oxazolyl, thiazolyl, imidazolyl, pyrazolyl, isoxazolyl, isothiazolyl, triazolyl, thiadiazolyl, pyridinyl, pyridazinyl, pyrimidinyl, pyrazinyl, triazinyl, quinolinyl, isoquinlinyl, benzimidazolyl, benzoxazolyl, benzothiazolyl, indolyl, isoindolyl, and benzothienyl.

A “heteroaralkyl” or “heteroarylalkyl” is heteroaryl group connected, as a substituent, via an alkylene group. Examples include but are not limited to 2-thienylmethyl, 3-thienylmethyl, furylmethyl, thienylethyl, pyrrolylalkyl, pyridylalkyl, isoxazollylalkyl, and imidazolylalkyl. In some cases, the alkylene group is a lower alkylene group (i.e., a C₁₋₄ alkylene group).

As used herein, “carbocyclyl” means a non-aromatic cyclic ring or ring system containing only carbon atoms in the ring system backbone. When the carbocyclyl is a ring system, two or more rings may be joined together in a fused, bridged or spiro-connected fashion. Carbocyclyls may have any degree of saturation provided that at least one ring in a ring system is not aromatic. Thus, carbocyclyls include cycloalkyls, cycloalkenyls, and cycloalkynyls. The carbocyclyl group may have 3 to 20 carbon atoms, although the present definition also covers the occurrence of the term “carbocyclyl” where no numerical range is designated. The carbocyclyl group may also be a medium size carbocyclyl having 3 to 10 carbon atoms. The carbocyclyl group could also be a carbocyclyl having 3 to 6 carbon atoms. The carbocyclyl group may be designated as “C₃₋₆ carbocyclyl” or similar designations. Examples of carbocyclyl rings include, but are not limited to, cyclopropyl, cyclobutyl, cyclopentyl, cyclohexyl, cyclohexenyl, 2,3-dihydro-indene, bicycle[2.2.2]octanyl, adamantyl, and spiro[4.4]nonanyl.

A “(carbocyclyl)alkyl” is a carbocyclyl group connected, as a substituent, via an alkylene group, such as “C₄₋₁₀ (carbocyclyl)alkyl” and the like, including but not limited to, cyclopropylmethyl, cyclobutylmethyl, cyclopropylethyl, cyclopropylbutyl, cyclobutylethyl, cyclopropylisopropyl, cyclopentylmethyl, cyclopentylethyl, cyclohexylmethyl, cyclohexylethyl, cycloheptylmethyl, and the like. In some cases, the alkylene group is a lower alkylene group.

As used herein, “cycloalkyl” means a fully saturated carbocyclyl ring or ring system. Examples include cyclopropyl, cyclobutyl, cyclopentyl, and cyclohexyl.

As used herein, “cycloalkenyl” means a carbocyclyl ring or ring system having at least one double bond, wherein no ring in the ring system is aromatic. An example is cyclohexenyl.

As used herein, “heterocyclyl” means a non-aromatic cyclic ring or ring system containing at least one heteroatom in the ring backbone. Heterocyclyls may be joined together in a fused, bridged or spiro-connected fashion. Heterocyclyls may have any degree of saturation provided that at least one ring in the ring system is not aromatic. The heteroatom(s) may be present in either a non-aromatic or aromatic ring in the ring system. The heterocyclyl group may have 3 to 20 ring members (i.e., the number of atoms making up the ring backbone, including carbon atoms and heteroatoms), although the present definition also covers the occurrence of the term “heterocyclyl” where no numerical range is designated. The heterocyclyl group may also be a medium size heterocyclyl having 3 to 10 ring members. The heterocyclyl group could also be a heterocyclyl having 3 to 6 ring members. The heterocyclyl group may be designated as “3-6 membered heterocyclyl” or similar designations. In preferred six membered monocyclic heterocyclyls, the heteroatom(s) are selected from one up to three of O, N or S, and in preferred five membered monocyclic heterocyclyls, the heteroatom(s) are selected from one or two heteroatoms selected from O, N, or S. Examples of heterocyclyl rings include, but are not limited to, azepinyl, acridinyl, carbazolyl, cinnolinyl, dioxolanyl, imidazolinyl, imidazolidinyl, morpholinyl, oxiranyl, oxepanyl, thiepanyl, piperidinyl, piperazinyl, dioxopiperazinyl, pyrrolidinyl, pyrrolidonyl, pyrrolidionyl, 4-piperidonyl, pyrazolinyl, pyrazolidinyl, 1,3-dioxinyl, 1,3-dioxanyl, 1,4-dioxinyl, 1,4-dioxanyl, 1,3-oxathianyl, 1,4-oxathiinyl, 1,4-oxathianyl, 2H-1,2-oxazinyl, trioxanyl, hexahydro-1,3,5-triazinyl, 1,3-dioxolyl, 1,3-dioxolanyl, 1,3-dithiolyl, 1,3-dithiolanyl, isoxazolinyl, isoxazolidinyl, oxazolinyl, oxazolidinyl, oxazolidinonyl, thiazolinyl, thiazolidinyl, 1,3-oxathiolanyl, indolinyl, isoindolinyl, tetrahydrofuranyl, tetrahydropyranyl, tetrahydrothiophenyl, tetrahydrothiopyranyl, tetrahydro-1,4-thiazinyl, thiamorpholinyl, dihydrobenzofuranyl, benzimidazolidinyl, and tetrahydroquinoline.

A “(heterocyclyl)alkyl” is a heterocyclyl group connected, as a substituent, via an alkylene group. Examples include, but are not limited to, imidazolinylmethyl and indolinylethyl.

As used herein, “acyl” refers to —C(═O)R, wherein R is hydrogen, C₁₋₆ alkyl, C₂₋₆ alkenyl, C₂₋₆ alkynyl, C₃₋₇ carbocyclyl, aryl, 5-10 membered heteroaryl, and 5-10 membered heterocyclyl, as defined herein. Non-limiting examples include formyl, acetyl, propanoyl, benzoyl, and acryl.

An “O-carboxy” group refers to a “—OC(═O)R” group in which R is selected from hydrogen, C₁₋₆ alkyl, C₂₋₆ alkenyl, C₂₋₆ alkynyl, C₃₋₇ carbocyclyl, aryl, 5-10 membered heteroaryl, and 5-10 membered heterocyclyl, as defined herein.

A “C-carboxy” group refers to a “—C(═O)OR” group in which R is selected from hydrogen, C₁₋₆ alkyl, C₂₋₆ alkenyl, C₂₋₆ alkynyl, C₃₋₇ carbocyclyl, aryl, 5-10 membered heteroaryl, and 5-10 membered heterocyclyl, as defined herein. A non-limiting example includes carboxyl (i.e., —C(═O)OH).

A “cyano” group refers to a “—CN” group.

A “cyanato” group refers to an “—OCN” group.

An “isocyanato” group refers to a “—NCO” group.

A “thiocyanato” group refers to a “—SCN” group.

An “isothiocyanato” group refers to an “—NCS” group.

A “sulfinyl” group refers to an “—S(═O)R” group in which R is selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

A “sulfonyl” group refers to an “—SO₂R” group in which R is selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

An “S-sulfonamido” group refers to a “—SO₂NR_{A}R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

An “N-sulfonamido” group refers to a “—N(R_{A})SO₂R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

An “O-carbamyl” group refers to a “—OC(═O)NR_{A}R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

An “N-carbamyl” group refers to an “—N(R_{A})OC(═O)R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

An “O-thiocarbamyl” group refers to a “—OC(═S)NR_{A}R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

An “N-thiocarbamyl” group refers to an “—N(R_{A})OC(═S)R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

A “C-amido” group refers to a “—C(═O)NR_{A}R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

An “N-amido” group refers to a “—N(R_{A})C(═O)R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

An “amino” group refers to a “—NR_{A}R_{B}” group in which R_{A}and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

An “aminoalkyl” group refers to an amino group connected via an alkylene group.

An “alkoxyalkyl” group refers to an alkoxy group connected via an alkylene group, such as a “C₂₋₈ alkoxyalkyl” and the like.

As used herein, a substituted group is derived from the unsubstituted parent group in which there has been an exchange of one or more hydrogen atoms for another atom or group. Unless otherwise indicated, when a group is deemed to be “substituted,” it is meant that the group is substituted with one or more subsitutents independently selected from C₁-C₆ alkyl, C₁-C₆ alkenyl, C₁-C₆ alkynyl, C₁-C₆ heteroalkyl, C₃-C₇ carbocyclyl (optionally substituted with halo, C₁-C₆ alkyl, C₁-C₆ alkoxy, C₁-C₆ haloalkyl, and C₁-C₆ haloalkoxy), C₃-C₇-carbocyclyl-C₁-C₆-alkyl (optionally substituted with halo, C₁-C₆ alkyl, C₁-C₆ alkoxy, C₁-C₆ haloalkyl, and C₁-C₆ haloalkoxy), 5-10 membered heterocyclyl (optionally substituted with halo, C₁-C₆ alkyl, C₁-C₆ alkoxy, C₁-C₆ haloalkyl, and C₁-C₆ haloalkoxy), 5-10 membered heterocyclyl-C₁-C₆-alkyl (optionally substituted with halo, C₁-C₆ alkyl, C₁-C₆ alkoxy, C₁-C₆ haloalkyl, and C₁-C₆ haloalkoxy), aryl (optionally substituted with halo, C₁-C₆ alkyl, C₁-C₆ alkoxy, C₁-C₆ haloalkyl, and C₁-C₆ haloalkoxy), aryl(C₁-C₆)alkyl (optionally substituted with halo, C₁-C₆ alkyl, C₁-C₆ alkoxy, C₁-C₆ haloalkyl, and C₁-C₆ haloalkoxy), 5-10 membered heteroaryl (optionally substituted with halo, C₁-C₆ alkyl, C₁-C₆ alkoxy, C₁-C₆ haloalkyl, and C₁-C₆ haloalkoxy), 5-10 membered heteroaryl(C₁-C₆)alkyl (optionally substituted with halo, C₁-C₆ alkyl, C₁-C₆ alkoxy, C₁-C₆ haloalkyl, and C₁-C₆ haloalkoxy), halo, cyano, hydroxy, C₁-C₆ alkoxy, C₁-C₆ alkoxy(C₁-C₆)alkyl (i.e., ether), aryloxy, sulfhydryl (mercapto), halo(C₁-C₆)alkyl (e.g., —CF₃), halo(C₁-C₆)alkoxy (e.g., —OCF₃), C₁-C₆ alkylthio, arylthio, amino, amino(C₁-C₆)alkyl, nitro, O-carbamyl, N-carbamyl, O-thiocarbamyl, N-thiocarbamyl, C-amido, N-amido, S-sulfonamido, N-sulfonamido, C-carboxy, O-carboxy, acyl, cyanato, isocyanato, thiocyanato, isothiocyanato, sulfinyl, sulfonyl, and oxo (═O). Wherever a group is described as “optionally substituted” that group can be substituted with the above substituents.

In some embodiments, substituted group(s) is (are) substituted with one or more substituent(s) individually and independently selected from C₁-C₄ alkyl, amino, hydroxy, and halogen.

It is to be understood that certain radical naming conventions can include either a mono-radical or a di-radical, depending on the context. For example, where a substituent requires two points of attachment to the rest of the molecule, it is understood that the substituent is a di-radical. For example, a substituent identified as alkyl that requires two points of attachment includes di-radicals such as —CH₂—, —CH₂CH₂—, —CH₂CH(CH₃)CH₂—, and the like. Other radical naming conventions clearly indicate that the radical is a di-radical such as “alkylene” or “alkenylene.”

As used herein, “alkylene” means a branched, or straight chain fully saturated di-radical chemical group containing only carbon and hydrogen that is attached to the rest of the molecule via two points of attachment (i.e., an alkanediyl). The alkylene group may have 1 to 20 carbon atoms, although the present definition also covers the occurrence of the term alkylene where no numerical range is designated. The alkylene group may also be a medium size alkylene having 1 to 9 carbon atoms. The alkylene group could also be a lower alkylene having 1 to 4 carbon atoms. The alkylene group may be designated as “C₁₋₄ alkylene” or similar designations. By way of example only, “C₁₋₄ alkylene” indicates that there are one to four carbon atoms in the alkylene chain, i.e., the alkylene chain is selected from the group consisting of methylene, ethylene, ethan-1,1-diyl, propylene, propan-1,1-diyl, propan-2,2-diyl, 1-methyl-ethylene, butylene, butan-1,1-diyl, butan-2,2-diyl, 2-methyl-propan-1,1-diyl, 1-methyl-propylene, 2-methyl-propylene, 1,1-dimethyl-ethylene, 1,2-dimethyl-ethylene, and 1-ethyl-ethylene.

As used herein, “alkenylene” means a straight or branched chain di-radical chemical group containing only carbon and hydrogen and containing at least one carbon-carbon double bond that is attached to the rest of the molecule via two points of attachment. The alkenylene group may have 2 to 20 carbon atoms, although the present definition also covers the occurrence of the term alkenylene where no numerical range is designated. The alkenylene group may also be a medium size alkenylene having 2 to 9 carbon atoms. The alkenylene group could also be a lower alkenylene having 2 to 4 carbon atoms. The alkenylene group may be designated as “C₂₋₄ alkenylene” or similar designations. By way of example only, “C₂₋₄ alkenylene” indicates that there are two to four carbon atoms in the alkenylene chain, i.e., the alkenylene chain is selected from the group consisting of ethenylene, ethen-1,1-diyl, propenylene, propen-1,1-diyl, prop-2-en-1,1-diyl, 1-methyl-ethenylene, but-1-enylene, but-2-enylene, but-1,3-dienylene, buten-1,1-diyl, but-1,3-dien-1,1-diyl, but-2-en-1,1-diyl, but-3-en-1,1-diyl, 1-methyl-prop-2-en-1,1-diyl, 2-methyl-prop-2-en-1,1-diyl, 1-ethyl-ethenylene, 1,2-dimethyl-ethenylene, 1-methyl-propenylene, 2-methyl-propenylene, 3-methyl-propenylene, 2-methyl-propen-1,1-diyl, and 2,2-dimethyl-ethen-1,1-diyl.

When two R groups are said to form a ring (e.g., a carbocyclyl, heterocyclyl, aryl, or heteroaryl ring) “together with the atom to which they are attached,” it is meant that the collective unit of the atom and the two R groups are the recited ring. The ring is not otherwise limited by the definition of each R group when taken individually. For example, when the following substructure is present:

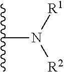

and R¹ and R² are defined as selected from the group consisting of hydrogen and alkyl, or R¹ and R² together with the nitrogen to which they are attached form a heterocyclyl, it is meant that R¹ and R² can be selected from hydrogen or alkyl, or alternatively, the substructure has structure:

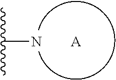

where ring A is a heterocyclyl ring containing the depicted nitrogen.

Similarly, when two “adjacent” R groups are said to form a ring “together with the atoms to which they are attached,” it is meant that the collective unit of the atoms, intervening bonds, and the two R groups are the recited ring. For example, when the following substructure is present:

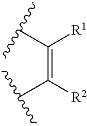

and R¹ and R² are defined as selected from the group consisting of hydrogen and alkyl, or R¹ and R² together with the atoms to which they are attached form an aryl or carbocylyl, it is meant that R¹ and R² can be selected from hydrogen or alkyl, or alternatively, the substructure has structure:

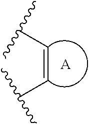

where A is an aryl ring or a carbocylyl containing the depicted double bond.

Wherever a substituent is depicted as a di-radical (i.e., has two points of attachment to the rest of the molecule), it is to be understood that the substituent can be attached in any directional configuration unless otherwise indicated. Thus, for example, a substituent depicted as —AE— or

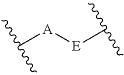

includes the substituent being oriented such that the A is attached at the leftmost attachment point of the molecule as well as the case in which A is attached at the rightmost attachment point of the molecule.

As used herein, “isosteres” of a chemical group are other chemical groups that exhibit the same or similar properties. For example, tetrazole is an isostere of carboxylic acid because it mimics the properties of carboxylic acid even though they both have very different molecular formulae. Tetrazole is one of many possible isosteric replacements for carboxylic acid. Other carboxylic acid isosteres contemplated include —SO₃H, —SO₂HNR, —PO₂(R)₂, —PO₃(R)₂, —CONHNHSO₂R, —COHNSO₂R, and —CONRCN, where R is selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 3-10 membered heterocyclyl, as defined herein. In addition, carboxylic acid isosteres can include 5-7 membered carbocycles or heterocycles containing any combination of CH₂, O, S, or N in any chemically stable oxidation state, where any of the atoms of said ring structure are optionally substituted in one or more positions. The following structures are non-limiting examples of carbocyclic and heterocyclic isosteres contemplated. The atoms of said ring structure may be optionally substituted at one or more positions with R as defined above.

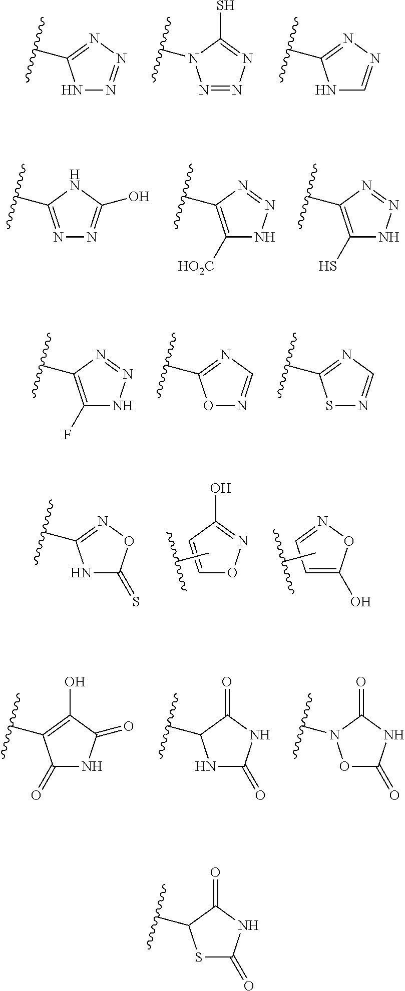

It is also contemplated that when chemical substituents are added to a carboxylic isostere, the compound retains the properties of a carboxylic isostere. It is contemplated that when a carboxylic isostere is optionally substituted with one or more moieties selected from R as defined above, then the substitution and substitution position is selected such that it does not eliminate the carboxylic acid isosteric properties of the compound. Similarly, it is also contemplated that the placement of one or more R substituents upon a carbocyclic or heterocyclic carboxylic acid isostere is not a substitution at one or more atom(s) that maintain(s) or is/are integral to the carboxylic acid isosteric properties of the compound, if such substituent(s) would destroy the carboxylic acid isosteric properties of the compound.

Other carboxylic acid isosteres not specifically exemplified in this specification are also contemplated.

The term “agent” or “test agent” includes any substance, molecule, element, compound, entity, or a combination thereof. It includes, but is not limited to, e.g., protein, polypeptide, peptide or mimetic, small organic molecule, polysaccharide, polynucleotide, and the like. It can be a natural product, a synthetic compound, or a chemical compound, or a combination of two or more substances. Unless otherwise specified, the terms “agent”, “substance”, and “compound” are used interchangeably herein.

The term “analog” is used herein to refer to a molecule that structurally resembles a reference molecule but which has been modified in a targeted and controlled manner, by replacing a specific substituent of the reference molecule with an alternate substituent. Compared to the reference molecule, an analog would be expected, by one skilled in the art, to exhibit the same, similar, or improved utility. Synthesis and screening of analogs, to identify variants of known compounds having improved characteristics (such as higher binding affinity for a target molecule) is an approach that is well known in pharmaceutical chemistry.

The term “mammal” is used in its usual biological sense. Thus, it specifically includes, but is not limited to, primates, including simians (chimpanzees, apes, monkeys) and humans, cattle, horses, sheep, goats, swine, rabbits, dogs, cats, rats and mice but also includes many other species.

The term “microbial infection” refers to the invasion of the host organism, whether the organism is a vertebrate, invertebrate, fish, plant, bird, or mammal, by pathogenic microbes. This includes the excessive growth of microbes that are normally present in or on the body of a mammal or other organism. More generally, a microbial infection can be any situation in which the presence of a microbial population(s) is damaging to a host mammal. Thus, a mammal is “suffering” from a microbial infection when excessive numbers of a microbial population are present in or on a mammal's body, or when the effects of the presence of a microbial population(s) is damaging the cells or other tissue of a mammal. Specifically, this description applies to a bacterial infection. Note that the compounds of preferred embodiments are also useful in treating microbial growth or contamination of cell cultures or other media, or inanimate surfaces or objects, and nothing herein should limit the preferred embodiments only to treatment of higher organisms, except when explicitly so specified in the claims.

The term “pharmaceutically acceptable carrier” or “pharmaceutically acceptable excipient” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. In addition, various adjuvants such as are commonly used in the art may be included. Considerations for the inclusion of various components in pharmaceutical compositions are described, e.g., in Gilman et al. (Eds.) (1990); Goodman and Gilman's: The Pharmacological Basis of Therapeutics, 8th Ed., Pergamon Press, which is incorporated herein by reference in its entirety.

“Subject” as used herein, means a human or a non-human mammal, e.g., a dog, a cat, a mouse, a rat, a cow, a sheep, a pig, a goat, a non-human primate or a bird, e.g., a chicken, as well as any other vertebrate or invertebrate.

An “effective amount” or a “therapeutically effective amount” as used herein refers to an amount of a therapeutic agent that is effective to relieve, to some extent, or to reduce the likelihood of onset of, one or more of the symptoms of a disease or condition, and includes curing a disease or condition. “Curing” means that the symptoms of a disease or condition are eliminated; however, certain long-term or permanent effects may exist even after a cure is obtained (such as extensive tissue damage).

“Treat,” “treatment,” or “treating,” as used herein refers to administering a pharmaceutical composition for prophylactic and/or therapeutic purposes. The term “prophylactic treatment” refers to treating a subject who does not yet exhibit symptoms of a disease or condition, but who is susceptible to, or otherwise at risk of, a particular disease or condition, whereby the treatment reduces the likelihood that the patient will develop the disease or condition. The term “therapeutic treatment” refers to administering treatment to a subject who exhibits symptoms of a disease or condition.

### Methods of Preparation

The compounds disclosed herein may be synthesized by methods described below, or by modification of these methods. Ways of modifying the methodology include, among others, temperature, solvent, reagents etc., known to those skilled in the art. In general, during any of the processes for preparation of the compounds disclosed herein, it may be necessary and/or desirable to protect sensitive or reactive groups on any of the molecules concerned. This may be achieved by means of conventional protecting groups, such as those described in Protective Groups in Organic Chemistry (ed. J. F. W. McOmie, Plenum Press, 1973); and P. G. M. Green, T. W. Wutts, Protecting Groups in Organic Synthesis (3rd ed.) Wiley, New York (1999), which are both hereby incorporated herein by reference in their entirety. The protecting groups may be removed at a convenient subsequent stage using methods known from the art. Synthetic chemistry transformations useful in synthesizing applicable compounds are known in the art and include e.g. those described in R. Larock, Comprehensive Organic Transformations, VCH Publishers, 1989, or L. Paquette, cd., Encyclopedia of Reagents for Organic Synthesis, John Wiley and Sons, 1995, which are both hereby incorporated herein by reference in their entirety. The routes shown and described herein are illustrative only and are not intended, nor are they to be construed, to limit the scope of the claims in any manner whatsoever. Those skilled in the art will be able to recognize modifications of the disclosed syntheses and to devise alternate routes based on the disclosures herein; all such modifications and alternate routes are within the scope of the claims.

In the following schemes, protecting groups for oxygen atoms are selected for their compatibility with the requisite synthetic steps as well as compatibility of the introduction and deprotection steps with the overall synthetic schemes (P. G. M. Green, T. W. Wutts, Protecting Groups in Organic Synthesis (3rd ed.) Wiley, New York (1999)). Handling of protecting and/or stereodirecting groups specific to boronic acid derivatives is described in a recent review of chemistry of boronic acids: D. G. Hall (Ed.), Boronic Acids. Preparation and Application in Organic Synthesis and Medicine, Wiley VCH (2005) and in earlier reviews: Matteson, D. S. (1988). Asymmetric synthesis with boronic esters. Accounts of Chemical Research, 21(8), 294-300, and Matteson, D. S. (1989). Tetrahedron, 45(7), 1859-1885), all of which are incorporated herein by reference in their entirety. The latter review articles also describe methodology for stereoselective insertion of halomethine functionality next to the boronate which is employed in the synthetic schemes below.

In addition to standard acid-catalyzed deprotection, special methods for removal of boronic acid protecting and/or stereodirecting groups include methods using fluorides (Yuen, A. K. L., & Hutton, C. A. (2005). Tetrahedron Letters, 46(46), 7899-7903—incorporated herein by reference in its entirety) or periodate oxidation (Coutts, S. J., et al. (1994). Tetrahedron Letters, 35(29), 5109-5112—incorporated herein by reference in its entirety) can also be employed in preparations of the compounds disclosed herein.

In strategies employing pinanediol or other diol-based chiral auxiliaries for stereospecific introduction of new chiral centers, the early stages of chemistry on boronic intermediates can be performed on chiral boronate esters or alternatively nonchiral borate/boronate intermediates can be used in early stages followed by transesterification with chiral diols prior to the step where stereoselection is required.

### Synthesis of Compounds of Formula I

The following example schemes are provided for the guidance of the reader, and collectively represent an example method for making the compounds encompassed herein. Furthermore, other methods for preparing compounds described herein will be readily apparent to the person of ordinary skill in the art in light of the following reaction schemes and examples. Unless otherwise indicated, all variables are as defined above.

Compounds of Formula (Ia) where R is H can be prepared as depicted in Schemes 1-11 from key intermediates V′, XIII′ and XX′ and XXVI′, which may be assembled by known reactions (Boronic Acids: Preparations and Applications in Organic Synthesis, Medicine and Materials, D. G. Hall, ed., Wiley-VCH, Weinheim, 2011, which is incorporated herein by reference in its entirety). Methods in the following section are defined for pure enantiomers of Formula (Ia). These methods are also applicable to make compounds of other enantiomer, Formula (Ic) or to make a racemic mixture by modifying the stereo-defining step.

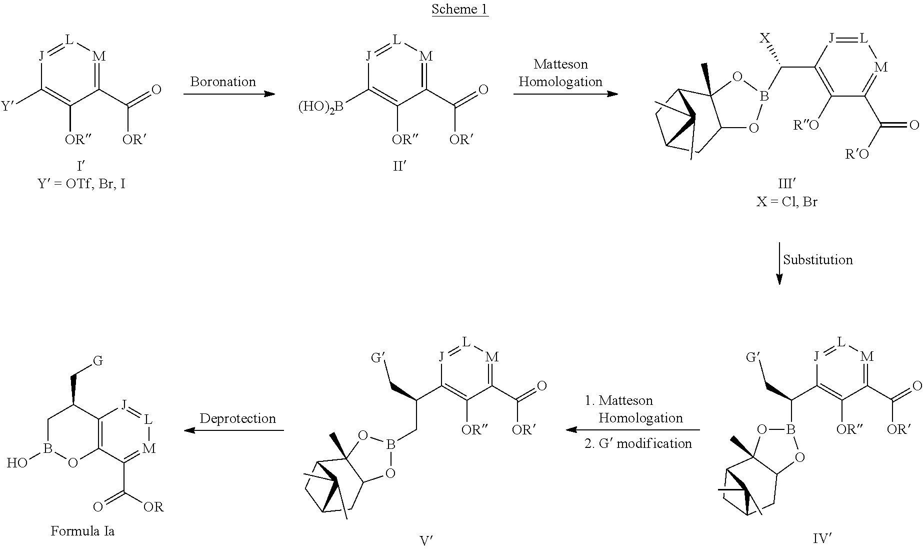

Compounds of Formula (Ia) can be made starting from protected aryl or heteroaryl intermediates of Formula II′ via a double Matteson homologation sequence (J. Org. Chem., 2013, 78, 10009-10023, which is incorporated herein by reference in its entirety). The compounds of Formula II′ may be attained from compounds of Formula I′ by means of several earlier known methods (WO0458679, which is incorporated herein by reference in its entirety) with conventional protecting groups for R′ and R″, such as those described in Protective Groups in Organic Chemistry (ed. J. F. W. McOmie, Plenum, 1973, which is incorporated herein by reference in its entirety); and Protecting Groups in Organic Synthesis P. G. M. Wutts, T. W. Green, Wiley, New York, 1999, which is incorporated herein by reference in its entirety) from commercially available salicylic acid derivatives. Aryl compounds of Formula I′ upon boronation by well-known available methods (Chem. Rev. 2010, 110, 890-931, which is incorporated herein by reference in its entirety) and boronate ester formation with desired chiral auxiliary give precursor for Matteson homologation. Compounds of Formula III′ where X=Cl and R′ is Boc and R″ is t-Butyl or R′ and R″ are protected together as isopropylidine or any other groups protected separately or together in cyclic form may be made from compounds of Formula II′ via homologation upon chloromethylene insertion with good stereocontrol by Matteson reaction conditions (WO0946098, which is incorporated herein by reference in its entirety). Compounds of Formula III′ where X is bromo may be made analogously to the chloro compounds of Scheme 1, utilizing dibromomethane (J. Am. Chem. Soc. 1990, 112, 3964-969, which is incorporated herein by reference in its entirety). The halo derivatives of Formula III′ where X is Cl or Br undergo stereospecific substitution by vinyl magnesium halide or allyl magnesium halide or enolate of t-butyl acetate (Tetrahedron 2005, 61, 4427-4536, which is incorporated herein by reference in its entirety), to give compounds of Formula IV′. These intermediates of Formula IV′ can be further treated to a non-substituted methylene homologation under Matteson reaction conditions. Such resulting intermediates can be further modified by conversion of G′ groups to G substitution. G′ groups such as vinyl or allyl functionalities can be converted to corresponding alcohols or acids or aldehydes by periodate oxidation or ozonolysis. Aldehyde functionalities can be converted to substituted amines by reductive amination to G-substituted compounds of Formula V′.

Simultaneous deprotection of pinane ester and salicylic acid protective groups of compounds of Formula V′ can be achieved by heating with dilute HCl, affording the desired compounds of Formula (Ia). This transformation may also be achieved by treatment with BCl₃ or BBr₃ (WO09064414, which is incorporated herein by reference in its entirety). Alternatively, the deprotection may be attained via trans-esterification with isobutyl boronic acid in presence of dilute acid (WO09064413, which is incorporated herein by reference in its entirety) or via other known methods (J. Org. Chem. (2010), 75, 468-471, which is incorporated herein by reference in its entirety).

Salicylic acid derivatives of Formula I′ where Y′ is a leaving group undergo coupling reaction with Reformatsky reagent of acetate in Negishi conditions to give intermediates of Formula VI′ where X′ is OR″′ (Tetrahedron, 2014, 1508-1515, J. Org. Chem., 2013, 78, 8250-8266, which is incorporated herein by reference in its entirety) (Scheme 2). Such intermediates may be alkylated with halomethylene boronate derivative VIIA, followed by modification of ester by selective hydrolysis and reduction to give compounds of Formula V′ in high stereoselectivity (J. Am. Chem. Soc., 2011, 133, 11936-11939, which is incorporated herein by reference in its entirety). Intermediates of Formula VI′ undergo methylenation to give derivatives of VII′ (J. Org. Chem., 1986, 51, 2981-2988, which is incorporated herein by reference in its entirety). Intermediates of Formula VII′ undergo asymmetric boronation in known conditions to give compounds of Formula V′ (J. Am. Chem. Soc., 2010, 132, 10630-10633, which is incorporated herein by reference in its entirety). Such asymmetric boronation may also feasible where X′ is-NOR¹. Intermediates of Formula V′ can be further transformed to compound of Formula (Ia) under the conditions described in Scheme 1.

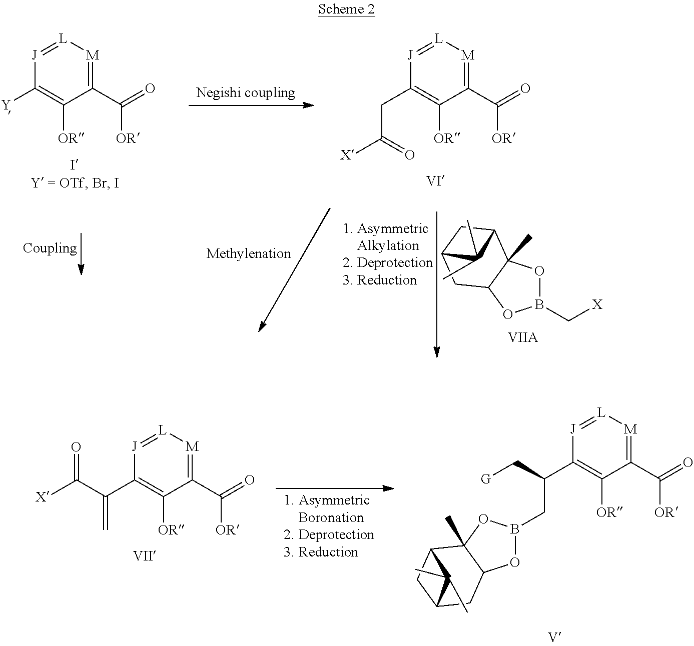

In an alternative sequence, compounds of Formula (Ia) can be made via boracarboxylation followed by asymmetric hydrogenation of acetylene intermediates of Formula VIII′ as shown in Scheme 3. Aryl or heteroaryl derivatives Formula I′ undergo a Pd-mediated coupling reaction to give an acetylene-substituted compound with TMS-acetylene. Boracarboxylation of alkynes with a diborane compound and carbon dioxide in presence of an N-heterocyclic carbene copper (I) complex as a catalyst gives α,β-unsaturated β-boralactone derivatives regio- and stereoselectively via a borylcupration/carboxylation (J. Am. Chem. Soc. 2012, 134, 14314-14317, which is incorporated herein by reference in its entirety). Such resulting derivatives can be transformed to esters of carboxylate and boronate to give intermediates of Formula IX′. Asymmetric hydrogenation of intermediates of Formula IX′ (Chem. Rev. 2003, 103, 3029-3070, which is incorporated herein by reference in its entirety) can be utilized to give enantiomerically pure compounds of Formula X′. Such compounds may be further transformed to compounds of Formula V′ by selective hydrolysis and reduction to give appropriate G substitution which on final deprotection gives compounds of Formula (Ia) via the steps as described above in Scheme 1.

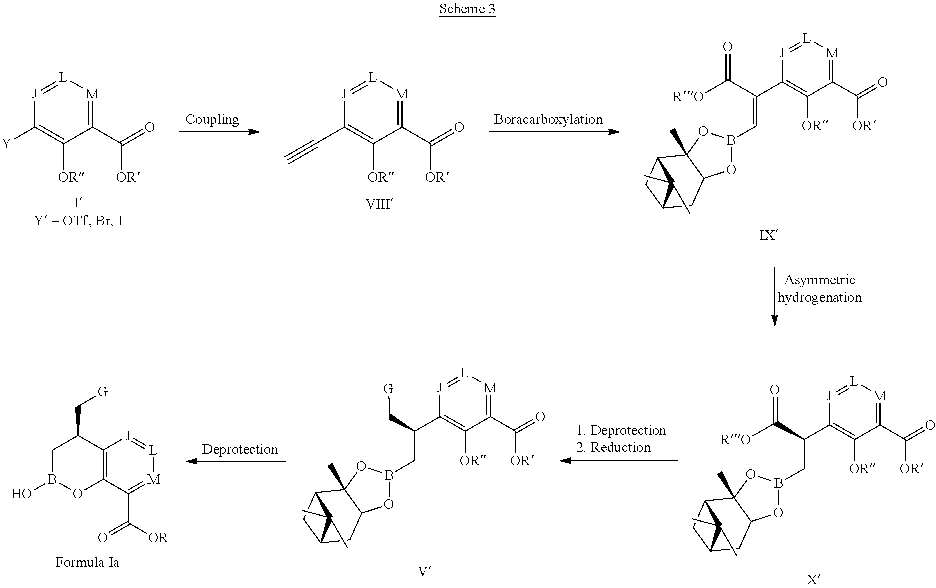

Compounds of Formula (Ia) where G=—NR¹C(O)R⁴, —NR¹C(O)NR¹R², or —NR¹C(O)OR³ may be prepared from carboxylic acid esters of Formula XI′ (obtained via compounds of Formula V′ where G is —CH₂CO₂^{t}Bu and R′ and R″ together are protected isopropylidene) as shown in Scheme 4. Such compounds may be converted to amides via selective t-Butyl ester hydrolysis and Curtius rearrangement (Chem. Rev. 1988, 88, 297-368; Org. Lett., 2005, 4107-4110, which are incorporated herein by reference in the entirety) followed by deprotection and amide formation to give compounds of Formula XIII′. Compounds of Formula XII′ may also be transformed to compounds of Formula (Ia) where G is —NHC(O)—O—R by hydrolysis.

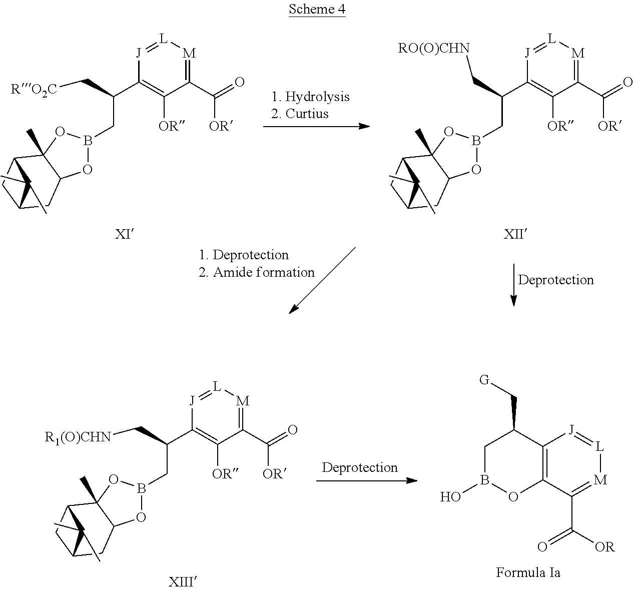

In an alternate route as shown in Scheme 5, compounds of Formula (Ia) can be obtained via intermediates of Formula XVII′. Such intermediates of Formula XVII′ can be assembled by asymmetric hydroboration (J. Am. Chem. Soc. 2014, 136, 15501-15504) and trans-esterification of 1,1′-disubstituted alkenes of Formula XVI′. Intermediates of Formula XVI′ can be obtained by coupling of substituted 2-bromo-propene derivatives with boronic acids of Formula XIV′ by palladium-catalyzed reactions. Intermediates of Formula XVII′ can be further transformed to compounds of Formula V′ by converting the G′ group to G (from esters to acids by selective hydrolysis followed by conversion to alcohols or amides) utilizing transformations shown in Schemes 3 and 4.

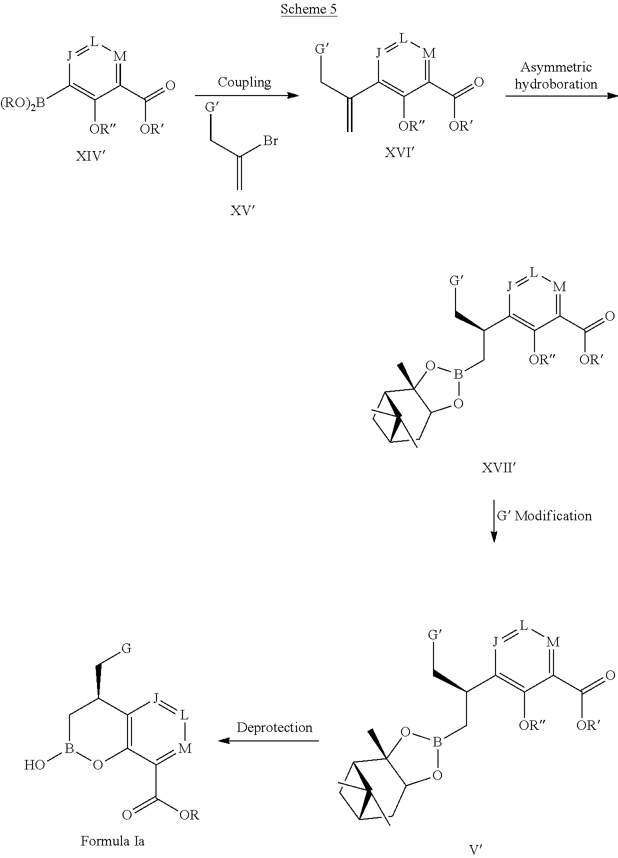

Compounds of Formula (Ia) can also be assembled in a convergent method as shown in Scheme 6 via intermediates of Formula XX′. Such intermediates of Formula XX′ are made by coupling of substituted diboronate precursors of Formula XIX′ (Org. Lett., 2014, 16, 6240-6243) with precursors of Formula I′ by palladium catalysis. Diboronates of Formula XIX′ are prepared from propargyl derivatives of Formula XVIII′ by utilizing an Ir-catalyzed method (J. Am. Chem. Soc., 2010, 132, 2548-2549). Such intermediates of Formula XX′ (where G′ can be —OTIPS or —CO₂Me or —CONR′R″ for further modification or deprotection) are known to undergo enantioselective hydrogenation (Angew. Chem. Int. Ed., 2011, 50, 1-6; Chem. Eur. J., 2012, 18, 6724-6728) to result in intermediates of Formula XXI′. Modification of G′ group to G and deprotection of XXI′, as described above leads to compounds of Formula (Ia).

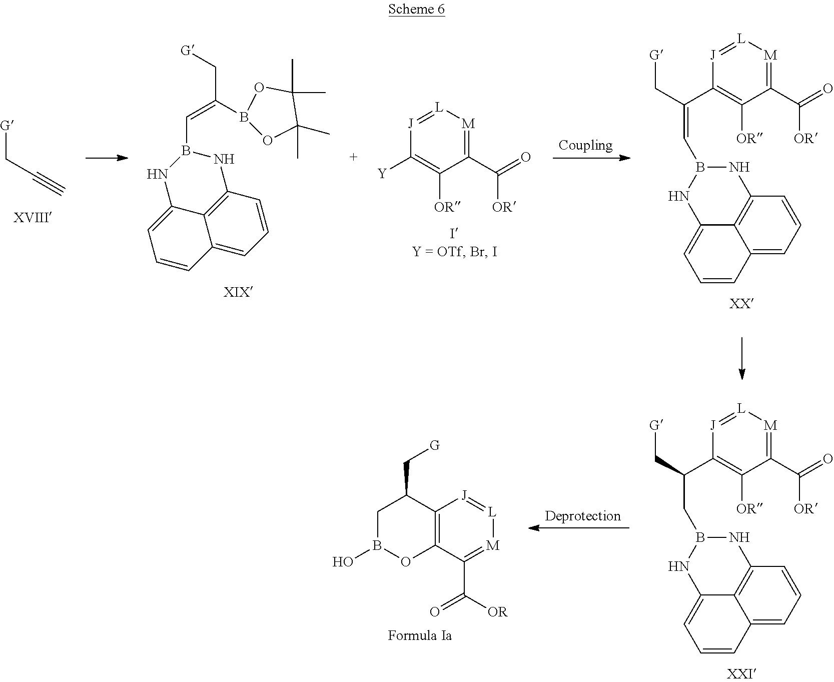

In an alternative synthetic route shown below, compounds of Formula (Ia) can be made via nickel-catalyzed boron-insertion (J. Am. Chem. Soc., 2016, 138, 15315-15318) of benzofuran derivatives of Formula XXV′ to give intermediates of Formula XXVI′. Such oxaborinane intermediates upon enantioselective hydrogenation (Angew. Chem. Int. Ed., 2011, 50, 1-6; Chem. Eur. J., 2012, 18, 6724-6728) followed by modification of G′ group and deprotection give compounds of Formula (Ia). Benzofuran derivatives of Formula XXV′ can be made by several known diverse methods including cyclization of intermediates of Formula XXIV′ (Org. Biomol. Chem., 2016, 14, 8074-8087). Such intermediates of Formula XXIV′ can be obtained via alkylation of appropriately substituted phenol derivatives of Formula XXIII′ with substituted bromoacetone derivatives of Formula XXII′ (Tetrahedron, 2013, 69, 5937-5944).

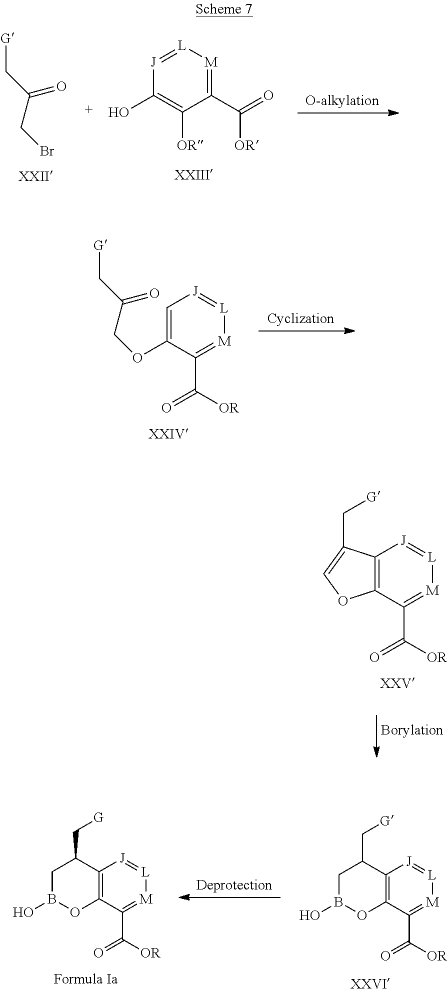

Intermediates of Formula XXIX′, which may alternatively be featured in routes to attain compounds of Formula (Ia) may be prepared as shown in Scheme 8. Such intermediates of Formula XXIX′ can be synthesized from compounds of Formula XXVII′ where X′ is a triflate, bromo, or iodo group by utilizing a Reformatsky reagent of a bromomethylene acetate ester (J. Org. Chem., 2013, 78, 8250-8266; Chem Lett., 1993, 845-848, which are incorporated herein by reference in the entirety). Compounds of Formula XXVIII′ where X′ is substituted with bromo or iodo groups can be attained from appropriately protected commercial 2,5-hydroxy-benzoic acid derivatives (J. Med. Chem., 2003, 46, 3437-3440, which is incorporated herein by reference in its entirety). Intermediates of Formula XXXVIII′ can also be prepared via carboxylation of derivatives of Formula XXXVII′ where Z′ is a fluoro or OR′ or SR′ by earlier described methods (WO12106995, which is incorporated herein by reference in its entirety).

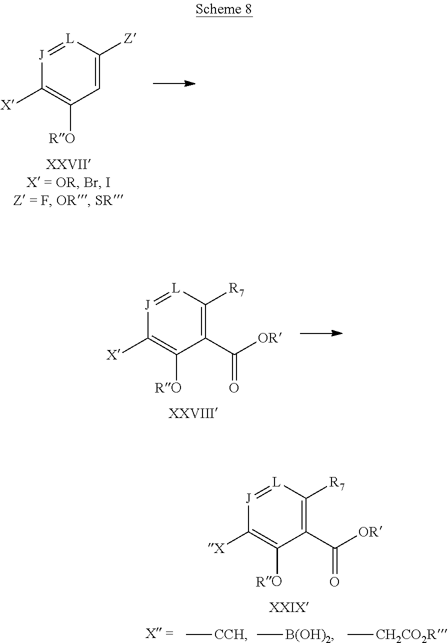

In another exemplary synthetic route, as shown in Scheme 9, the compounds of Formula XXXII′ can be prepared from a salicylic acid derivative of a compound of Formula XXXI′. The compounds of Formula XXX′ upon diallylation under basic conditions followed by thermal Claisen rearrangement (Org. React. 1975, 22, 1-252, which is incorporated herein by reference in its entirety) and ester hydrolysis give compounds of Formula XXXI′. Such compounds upon protection and oxidation followed by esterification result in phenylacetic acid derivatives of Formula XXXII′. Compounds of Formula XXXII′ can be further transformed as shown above in Scheme 2. The compound of formula XXX′ can also undergo the steps listed above in Scheme 8 to form an ortho-carboxylate-substituted compound of Formula XXIX′.

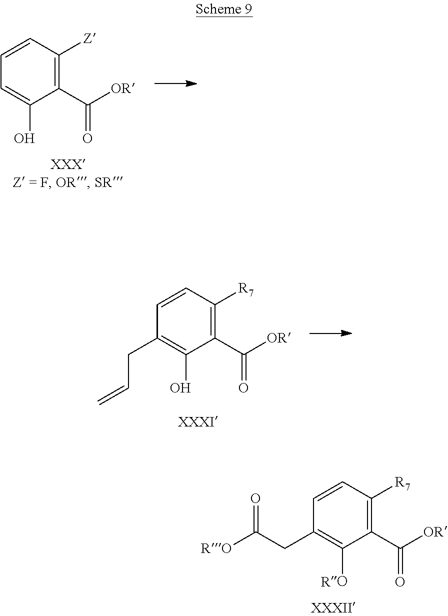

### Synthesis of Prodrugs

Compounds of Formula (Ia) where the R is a prodrug moiety may be synthesized by a variety of known methods of producing different carboxylic acid prodrugs (Prodrugs: Challenges and Rewards, V. J. Stella, et al., ed., Springer, New York, 2007, which is incorporated herein by reference in its entirety). These prodrugs include but are not limited to substituted or non-substituted alkyl esters, (acyloxy)alkyl esters (Synthesis 2012, 44, 207, which is incorporated herein by reference in its entirety), [(alkoxycarbonyl)oxy]methyl esters (WO10097675, which is incorporated herein by reference in its entirety), or (oxodioxolyl)methyl esters (J. Med. Chem. 1996, 39, 323-338, which is incorporated herein by reference in its entirety). Such prodrugs can be made from compounds of Formula (Ia) where R=H (Formula XXXIII′) by treatment with acid or in neutral conditions (e.g., carbodiimide coupling) in the presence of alcohols (ROH) or via base-promoted esterification with RX where X is a leaving group in the presence of an appropriate base.

One exemplary but non-limiting general synthetic route for preparing prodrugs is shown in Scheme 10 below. The boronic acid of Formula XXXIII′ can react with a chloro- or bromo-substituted prodrug moiety to form a prodrug of Formula (Ia) where R is a prodrug moiety. Examples of the prodrug moiety R can be —C₁₋₉alkyl, —CR⁹R¹⁰OC(O)C₁₋₉alkyl, —CR⁹R¹⁰OC(O)OC₁₋₉alkyl, —CR⁹R¹⁰OC(O) C₃₋₇ carbocyclyl, —CR⁹R¹⁰OC(O)O C₃₋₇ carbocyclyl; —CR⁹R¹⁰OC(O)(5-10 membered heterocyclyl), —CR⁹R¹⁰OC(O)O(5-10 membered heterocyclyl), and

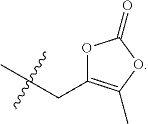 [SMILES: *Cc1oc(=O)oc1C]

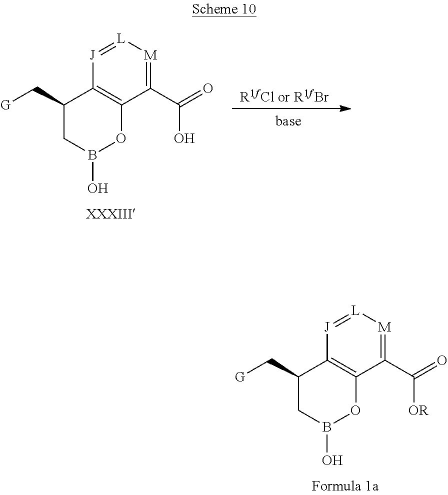

Alternatively, boronate esters of Formula XXXIV′ or corresponding trifluoroborates (Chem. Rev. 2008, 108, 288-325, which is incorporated herein by reference in its entirety) may be also utilized for introduction of prodrugs and to convert them to final prodrugs (Scheme 11). Such carboxylic acids (XXXIV′) can be made from compounds of Formula V′ by selective deprotection of OR′. The prodrug group may also be introduced earlier in the sequence in compounds of Formula IV′ where R′is R. Such a sequence in which a prodrug is introduced in earlier intermediates is only feasible when the ester is stable enough under the final deprotection conditions to remove the phenol protective group and boronate ester group.

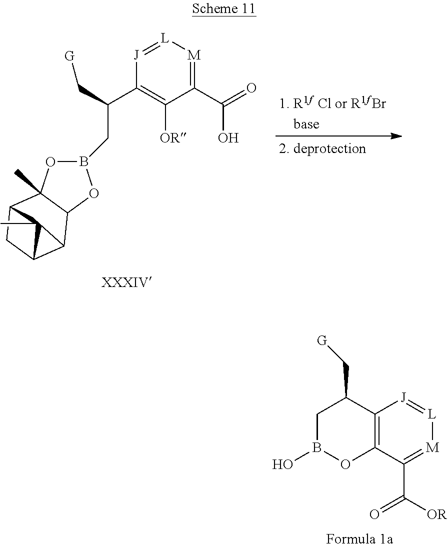

### Administration and Pharmaceutical Compositions

The compounds are administered at a therapeutically effective dosage. While human dosage levels have yet to be optimized for the compounds described herein, generally, a daily dose may be from about 0.25 mg/kg to about 120 mg/kg or more of body weight, from about 0.5 mg/kg or less to about 70 mg/kg, from about 1.0 mg/kg to about 50 mg/kg of body weight, or from about 1.5 mg/kg to about 10 mg/kg of body weight. Thus, for administration to a 70 kg person, the dosage range would be from about 17 mg per day to about 8000 mg per day, from about 35 mg per day or less to about 7000 mg per day or more, from about 70 mg per day to about 6000 mg per day, from about 100 mg per day to about 5000 mg per day, or from about 200 mg to about 3000 mg per day. The amount of active compound administered will, of course, be dependent on the subject and disease state being treated, the severity of the affliction, the manner and schedule of administration and the judgment of the prescribing physician.

Administration of the compounds disclosed herein or the pharmaceutically acceptable salts thereof can be via any of the accepted modes of administration for agents that serve similar utilities including, but not limited to, orally, subcutaneously, intravenously, intranasally, topically, transdermally, intraperitoneally, intramuscularly, intrapulmonarilly, vaginally, rectally, or intraocularly. Oral and parenteral administrations are customary in treating the indications that are the subject of the preferred embodiments.

The compounds useful as described above can be formulated into pharmaceutical compositions for use in treatment of these conditions. Standard pharmaceutical formulation techniques are used, such as those disclosed in Remington's The Science and Practice of Pharmacy, 21st Ed., Lippincott Williams & Wilkins (2005), incorporated by reference in its entirety. Accordingly, some embodiments include pharmaceutical compositions comprising: (a) a safe and therapeutically effective amount of a compound described herein (including enantiomers, diastereoisomers, tautomers, polymorphs, and solvates thereof), or pharmaceutically acceptable salts thereof; and (b) a pharmaceutically acceptable carrier, diluent, excipient or combination thereof.

In addition to the selected compound useful as described above, come embodiments include compositions containing a pharmaceutically-acceptable carrier. The term “pharmaceutically acceptable carrier” or “pharmaceutically acceptable excipient” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. In addition, various adjuvants such as are commonly used in the art may be included. Considerations for the inclusion of various components in pharmaceutical compositions are described, e.g., in Gilman et al. (Eds.) (1990); Goodman and Gilman's: The Pharmacological Basis of Therapeutics, 8th Ed., Pergamon Press, which is incorporated herein by reference in its entirety.

Some examples of substances, which can serve as pharmaceutically-acceptable carriers or components thereof, are sugars, such as lactose, glucose and sucrose; starches, such as corn starch and potato starch; cellulose and its derivatives, such as sodium carboxymethyl cellulose, ethyl cellulose, and methyl cellulose; powdered tragacanth; malt; gelatin; talc; solid lubricants, such as stearic acid and magnesium stearate; calcium sulfate; vegetable oils, such as peanut oil, cottonseed oil, sesame oil, olive oil, corn oil and oil of theobroma; polyols such as propylene glycol, glycerine, sorbitol, mannitol, and polyethylene glycol; alginic acid; emulsifiers, such as the TWEENS; wetting agents, such sodium lauryl sulfate; coloring agents; flavoring agents; tableting agents, stabilizers; antioxidants; preservatives; pyrogen-free water; isotonic saline; and phosphate buffer solutions.

The choice of a pharmaceutically-acceptable carrier to be used in conjunction with the subject compound is basically determined by the way the compound is to be administered.

The compositions described herein are preferably provided in unit dosage form. As used herein, a “unit dosage form” is a composition containing an amount of a compound that is suitable for administration to an animal, preferably mammal subject, in a single dose, according to good medical practice. The preparation of a single or unit dosage form however, does not imply that the dosage form is administered once per day or once per course of therapy. Such dosage forms are contemplated to be administered once, twice, thrice or more per day and may be administered as infusion over a period of time (e.g., from about 30 minutes to about 2-6 hours), or administered as a continuous infusion, and may be given more than once during a course of therapy, though a single administration is not specifically excluded. The skilled artisan will recognize that the formulation does not specifically contemplate the entire course of therapy and such decisions are left for those skilled in the art of treatment rather than formulation.

The compositions useful as described above may be in any of a variety of suitable forms for a variety of routes for administration, for example, for oral, nasal, rectal, topical (including transdermal), ocular, intracerebral, intracranial, intrathecal, intra-arterial, intravenous, intramuscular, or other parental routes of administration. The skilled artisan will appreciate that oral and nasal compositions comprise compositions that are administered by inhalation, and made using available methodologies. Depending upon the particular route of administration desired, a variety of pharmaceutically-acceptable carriers well-known in the art may be used. Pharmaceutically-acceptable carriers include, for example, solid or liquid fillers, diluents, hydrotropics, surface-active agents, and encapsulating substances. Optional pharmaceutically-active materials may be included, which do not substantially interfere with the inhibitory activity of the compound. The amount of carrier employed in conjunction with the compound is sufficient to provide a practical quantity of material for administration per unit dose of the compound. Techniques and compositions for making dosage forms useful in the methods described herein are described in the following references, all incorporated by reference herein: Modern Pharmaceutics, 4th Ed., Chapters 9 and 10 (Banker & Rhodes, editors, 2002); Lieberman et al., Pharmaceutical Dosage Forms: Tablets (1989); and Ansel, Introduction to Pharmaceutical Dosage Forms 8th Edition (2004).

Various oral dosage forms can be used, including such solid forms as tablets, capsules, granules and bulk powders. Tablets can be compressed, tablet triturates, enteric-coated, sugar-coated, film-coated, or multiple-compressed, containing suitable binders, lubricants, diluents, disintegrating agents, coloring agents, flavoring agents, flow-inducing agents, and melting agents. Liquid oral dosage forms include aqueous solutions, emulsions, suspensions, solutions and/or suspensions reconstituted from non-effervescent granules, and effervescent preparations reconstituted from effervescent granules, containing suitable solvents, preservatives, emulsifying agents, suspending agents, diluents, sweeteners, melting agents, coloring agents and flavoring agents.

The pharmaceutically-acceptable carrier suitable for the preparation of unit dosage forms for peroral administration is well-known in the art. Tablets typically comprise conventional pharmaceutically-compatible adjuvants as inert diluents, such as calcium carbonate, sodium carbonate, mannitol, lactose and cellulose; binders such as starch, gelatin and sucrose; disintegrants such as starch, alginic acid and croscarmelose; lubricants such as magnesium stearate, stearic acid and talc. Glidants such as silicon dioxide can be used to improve flow characteristics of the powder mixture. Coloring agents, such as the FD&C dyes, can be added for appearance. Sweeteners and flavoring agents, such as aspartame, saccharin, menthol, peppermint, and fruit flavors, are useful adjuvants for chewable tablets. Capsules typically comprise one or more solid diluents disclosed above. The selection of carrier components depends on secondary considerations like taste, cost, and shelf stability, which are not critical, and can be readily made by a person skilled in the art.

Peroral compositions also include liquid solutions, emulsions, suspensions, and the like. The pharmaceutically-acceptable carriers suitable for preparation of such compositions are well known in the art. Typical components of carriers for syrups, elixirs, emulsions and suspensions include ethanol, glycerol, propylene glycol, polyethylene glycol, liquid sucrose, sorbitol and water. For a suspension, typical suspending agents include methyl cellulose, sodium carboxymethyl cellulose, AVICEL RC-591, tragacanth and sodium alginate; typical wetting agents include lecithin and polysorbate 80; and typical preservatives include methyl paraben and sodium benzoate. Peroral liquid compositions may also contain one or more components such as sweeteners, flavoring agents and colorants disclosed above.

Such compositions may also be coated by conventional methods, typically with pH or time-dependent coatings, such that the subject compound is released in the gastrointestinal tract in the vicinity of the desired topical application, or at various times to extend the desired action. Such dosage forms typically include, but are not limited to, one or more of cellulose acetate phthalate, polyvinylacetate phthalate, hydroxypropyl methyl cellulose phthalate, ethyl cellulose, Eudragit coatings, waxes and shellac.

Compositions described herein may optionally include other drug actives.

Other compositions useful for attaining systemic delivery of the subject compounds include sublingual, buccal and nasal dosage forms. Such compositions typically comprise one or more of soluble filler substances such as sucrose, sorbitol and mannitol; and binders such as acacia, microcrystalline cellulose, carboxymethyl cellulose and hydroxypropyl methyl cellulose. Glidants, lubricants, sweeteners, colorants, antioxidants and flavoring agents disclosed above may also be included.

A liquid composition, which is formulated for topical ophthalmic use, is formulated such that it can be administered topically to the eye. The comfort should be maximized as much as possible, although sometimes formulation considerations (e.g. drug stability) may necessitate less than optimal comfort. In the case that comfort cannot be maximized, the liquid should be formulated such that the liquid is tolerable to the patient for topical ophthalmic use. Additionally, an ophthalmically acceptable liquid should either be packaged for single use, or contain a preservative to prevent contamination over multiple uses.

For ophthalmic application, solutions or medicaments are often prepared using a physiological saline solution as a major vehicle. Ophthalmic solutions should preferably be maintained at a comfortable pH with an appropriate buffer system. The formulations may also contain conventional, pharmaceutically acceptable preservatives, stabilizers and surfactants.

Preservatives that may be used in the pharmaceutical compositions disclosed herein include, but are not limited to, benzalkonium chloride, PHMB, chlorobutanol, thimerosal, phenylmercuric, acetate and phenylmercuric nitrate. A useful surfactant is, for example, Tween 80. Likewise, various useful vehicles may be used in the ophthalmic preparations disclosed herein. These vehicles include, but are not limited to, polyvinyl alcohol, povidone, hydroxypropyl methyl cellulose, poloxamers, carboxymethyl cellulose, hydroxyethyl cellulose and purified water.

Tonicity adjustors may be added as needed or convenient. They include, but are not limited to, salts, particularly sodium chloride, potassium chloride, mannitol and glycerin, or any other suitable ophthalmically acceptable tonicity adjustor.

Various buffers and means for adjusting pH may be used so long as the resulting preparation is ophthalmically acceptable. For many compositions, the pH will be between 4 and 9. Accordingly, buffers include acetate buffers, citrate buffers, phosphate buffers and borate buffers. Acids or bases may be used to adjust the pH of these formulations as needed.

In a similar vein, an ophthalmically acceptable antioxidant includes, but is not limited to, sodium metabisulfite, sodium thiosulfate, acetylcysteine, butylated hydroxyanisole and butylated hydroxytoluene.

Other excipient components, which may be included in the ophthalmic preparations, are chelating agents. A useful chelating agent is edetate disodium, although other chelating agents may also be used in place or in conjunction with it.

For topical use, creams, ointments, gels, solutions or suspensions, etc., containing the compound disclosed herein are employed. Topical formulations may generally be comprised of a pharmaceutical carrier, co-solvent, emulsifier, penetration enhancer, preservative system, and emollient.

For intravenous administration, the compounds and compositions described herein may be dissolved or dispersed in a pharmaceutically acceptable diluent, such as a saline or dextrose solution. Suitable excipients may be included to achieve the desired pH, including but not limited to NaOH, sodium carbonate, sodium acetate, HCl, and citric acid. In various embodiments, the pH of the final composition ranges from 2 to 8, or preferably from 4 to 7. Antioxidant excipients may include sodium bisulfite, acetone sodium bisulfite, sodium formaldehyde, sulfoxylate, thiourea, and EDTA. Other non-limiting examples of suitable excipients found in the final intravenous composition may include sodium or potassium phosphates, citric acid, tartaric acid, gelatin, and carbohydrates such as dextrose, mannitol, and dextran. Further acceptable excipients are described in Powell, et al., Compendium of Excipients for Parenteral Formulations, PDA J Pharm Sci and Tech 1998, 52 238-311 and Nema et al., Excipients and Their Role in Approved Injectable Products: Current Usage and Future Directions, PDA J Pharm Sci and Tech 2011, 65 287-332, both of which are incorporated herein by reference in their entirety. Antimicrobial agents may also be included to achieve a bacteriostatic or fungistatic solution, including but not limited to phenylmercuric nitrate, thimerosal, benzethonium chloride, benzalkonium chloride, phenol, cresol, and chlorobutanol.

The compositions for intravenous administration may be provided to caregivers in the form of one more solids that are reconstituted with a suitable diluent such as sterile water, saline or dextrose in water shortly prior to administration. In other embodiments, the compositions are provided in solution ready to administer parenterally. In still other embodiments, the compositions are provided in a solution that is further diluted prior to administration. In embodiments that include administering a combination of a compound described herein and another agent, the combination may be provided to caregivers as a mixture, or the caregivers may mix the two agents prior to administration, or the two agents may be administered separately.

The actual dose of the active compounds described herein depends on the specific compound, and on the condition to be treated; the selection of the appropriate dose is well within the knowledge of the skilled artisan.

### Methods of Treatment

Some embodiments of the present invention include methods of treating bacterial infections with the compounds and compositions comprising the compounds described herein. Some methods include administering a compound, composition, pharmaceutical composition described herein to a subject in need thereof. In some embodiments, a subject can be an animal, e.g., a mammal (including a human). In some embodiments, the bacterial infection comprises a bacterium described herein. As will be appreciated from the foregoing, methods of treating a bacterial infection include methods for preventing bacterial infection in a subject at risk thereof.

In some embodiments, the subject is a human.

Further embodiments include administering a combination of compounds to a subject in need thereof. A combination can include a compound, composition, pharmaceutical composition described herein with an additional medicament.

Some embodiments include co-administering a compound, composition, and/or pharmaceutical composition described herein, with an additional medicament. By “co-administration,” it is meant that the two or more agents may be found in the patient's bloodstream at the same time, regardless of when or how they are actually administered. In one embodiment, the agents are administered simultaneously. In one such embodiment, administration in combination is accomplished by combining the agents in a single dosage form. In another embodiment, the agents are administered sequentially. In one embodiment, the agents are administered through the same route, such as orally. In another embodiment, the agents are administered through different routes, such as one being administered orally and another being administered intravenously.

Examples of additional medicaments include an antibacterial agent, antifungal agent, an antiviral agent, an anti-inflammatory agent and an anti-allergic agent.

Preferred embodiments include combinations of a compound, composition or pharmaceutical composition described herein with an antibacterial agent such as a β-lactam. Examples of such β-lactams include Amoxicillin, Ampicillin (e.g., Pivampicillin, Hetacillin, Bacampicillin, Metampicillin, Talampicillin), Epicillin, Carbenicillin (Carindacillin), Ticarcillin, Temocillin, Azlocillin, Piperacillin, Mezlocillin, Mecillinam (Pivmecillinam), Sulbenicillin, Benzylpenicillin (G), Clometocillin, Benzathine benzylpenicillin, Procaine benzylpenicillin, Azidocillin, Penamecillin, Phenoxymethylpenicillin (V), Propicillin, Benzathine phenoxymethylpenicillin, Pheneticillin, Cloxacillin (e.g., Dicloxacillin, Flucloxacillin), Oxacillin, Methicillin, Nafcillin, Faropenem, Biapenem, Doripenem, Ertapenem, Imipenem, Meropenem, Panipenem, Cefazolin, Cefacetrile, Cefadroxil, Cefalexin, Cefaloglycin, Cefalonium, Cefaloridine, Cefalotin, Cefapirin, Cefatrizine, Cefazedone, Cefazaflur, Cefradine, Cefroxadine, Ceftezole, Cefaclor, Cefamandole, Cefminox, Cefonicid, Ceforanide, Cefotiam, Cefprozil, Cefbuperazone, Cefuroxime, Cefuzonam, Cefoxitin, Cefotetan, Cefmetazole, Loracarbef, Cefixime, Ceftazidime, Ceftriaxone, Cefcapene, Cefdaloxime, Cefdinir, Cefditoren, Cefetamet, Cefmenoxime, Cefodizime, Cefoperazone, Cefotaxime, Cefpimizole, Cefpiramide, Cefpodoxime, Cefsulodin, Cefteram, Ceftibuten, Ceftiolene, Ceftizoxime, Flomoxef, Latamoxef, Cefepime, Cefozopran, Cefpirome, Cefquinome, Ceftobiprole, Ceftaroline, Ceftiofur, Cefquinome, Cefovecin, Aztreonam, Tigemonam, and Carumonam.

Preferred embodiments include β-lactams such as Ceftazidime, Biapenem, Doripenem, Ertapenem, Imipenem, Meropenem, Tebipenem, Tebipenem pivoxil, Apapenem, and Panipenem.

Additional preferred embodiments include β-lactams such as Aztreonam, Tigemonam, and Carumonam.

Some embodiments include a combination of the compounds, compositions and/or pharmaceutical compositions described herein with an additional agent, wherein the additional agent comprises a monobactam. Examples of monobactams include aztreonam, tigemonam, nocardicin A, carumonam, and tabtoxin. In some such embodiments, the compound, composition and/or pharmaceutical composition comprises a class A, C, or D β-lactamase inhibitor. Some embodiments include co-administering the compound, composition or pharmaceutical composition described herein with one or more additional agents.

Some embodiments include a combination of the compounds, compositions and/or pharmaceutical compositions described herein with an additional agent, wherein the additional agent comprises a class B β-lactamase inhibitor. An example of a class B β-lactamase inhibitor includes ME1071 (Yoshikazu Ishii et al, “In Vitro Potentiation of Carbapenems with ME1071, a Novel Metallo-β-Lactamase Inhibitor, against Metallo-β-lactamase Producing Pseudomonas aeruginosa Clinical Isolates.” Antimicrob. Agents Chemother. doi:10.1128/AAC.01397-09 (July 2010)). Some embodiments include co-administering the compound, composition or pharmaceutical composition described herein with one or more additional agents.

Some embodiments include a combination of the compounds, compositions and/or pharmaceutical compositions described herein with an additional agent, wherein the additional agent comprises one or more agents that include a class A, B, C, or D β-lactamase inhibitor. Some embodiments include co-administering the compound, composition or pharmaceutical composition described herein with the one or more additional agents.

### Indications

The compounds and compositions comprising the compounds described herein can be used to treat bacterial infections. Bacterial infections that can be treated with the compounds, compositions and methods described herein can comprise a wide spectrum of bacteria. Example organisms include gram-positive bacteria, gram-negative bacteria, aerobic and anaerobic bacteria, such as Staphylococcus, Lactobacillus, Streptococcus, Sarcina, Escherichia, Enterobacter, Klebsiella, Pseudomonas, Acinetobacter, Mycobacterium, Proteus, Campylobacter, Citrobacter, Nisseria, Baccillus, Bacteroides, Peptococcus, Clostridium, Salmonella, Shigella, Serratia, Haemophilus, Brucella, and other organisms.

More examples of bacterial infections include Pseudomonas aeruginosa, Pseudomonas fluorescens, Pseudomonas acidovorans, Pseudomonas alcaligenes, Pseudomonas putida, Stenotrophomonas maltophilia, Burkholderia cepacia, Aeromonas hydrophilia, Escherichia coli, Citrobacter freundii, Salmonella typhimurium, Salmonella typhi, Salmonella paratyphi, Salmonella enteritidis, Shigella dysenteriae, Shigella flexneri, Shigella sonnei, Enterobacter cloacae, Enterobacter aerogenes, Klebsiella pneumoniae, Klebsiella oxytoca, Serratia marcescens, Francisella tularensis, Morganella morganii, Proteus mirabilis, Proteus vulgaris, Providencia alcalifaciens, Providencia rettgeri, Providencia stuartii, Acinetobacter baumannii, Acinetobacter calcoaceticus, Acinetobacter haemolyticus, Yersinia enterocolitica, Yersinia pestis, Yersinia pseudotuberculosis, Yersinia intermedia, Bordetella pertussis, Bordetella parapertussis, Bordetella bronchiseptica, Haemophilus influenzae, Haemophilus parainfluenzae, Haemophilus haemolyticus, Haemophilus parahaemolyticus, Haemophilus ducreyi, Pasteurella multocida, Pasteurella haemolytica, Branhamella catarrhalis, Helicobacter pylori, Campylobacter fetus, Campylobacter jejuni, Campylobacter coli, Borrelia burgdorferi, Vibrio cholerae, Vibrio parahaemolyticus, Legionella pneumophila, Listeria monocytogenes, Neisseria gonorrhoeae, Neisseria meningitidis, Kingella, Moraxella, Gardnerella vaginalis, Bacteroides fragilis, Bacteroides distasonis, Bacteroides 3452A homology group, Bacteroides vulgatus, Bacteroides ovalus, Bacteroides thetaiotaomicron, Bacteroides uniformis, Bacteroides eggerthii, Bacteroides splanchnicus, Clostridium difficile, Mycobacterium tuberculosis, Mycobacterium avium, Mycobacterium intracellulare, Mycobacterium leprae, Corynebacterium diphtheriae, Corynebacterium ulcerans, Streptococcus pneumoniae, Streptococcus agalactiae, Streptococcus pyogenes, Enterococcus faecalis, Enterococcus faecium, Staphylococcus aureus, Staphylococcus epidermidis, Staphylococcus saprophyticus, Staphylococcus intermedius, Staphylococcus hyicus subsp. hyicus, Staphylococcus haemolyticus, Staphylococcus hominis, or Staphylococcus saccharolyticus.

To further illustrate this invention, the following examples are included. The examples should not, of course, be construed as specifically limiting the invention. Variations of these examples within the scope of the claims are within the purview of one skilled in the art and are considered to fall within the scope of the invention as described, and claimed herein. The reader will recognize that the skilled artisan, armed with the present disclosure, and skill in the art is able to prepare and use the invention without exhaustive examples. The following examples will further describe the present invention, and are used for the purposes of illustration only, and should not be considered as limiting.

### EXAMPLES

### General Procedures

Materials used in preparing the cyclic boronic acid ester derivatives described herein may be made by known methods or are commercially available. It will be apparent to the skilled artisan that methods for preparing precursors and functionality related to the compounds claimed herein are generally described in the literature including, for example, procedures described in U.S. Pat. No. 7,271,186 and WO2009064414, each of which is incorporated by reference in its entirety. In these reactions, it is also possible to make use of variants which are themselves known to those of ordinary skill in this art, but are not mentioned in greater detail. The skilled artisan given the literature and this disclosure is well equipped to prepare any of the compounds.

It is recognized that the skilled artisan in the art of organic chemistry can readily carry out manipulations without further direction, that is, it is well within the scope and practice of the skilled artisan to carry out these manipulations. These include reduction of carbonyl compounds to their corresponding alcohols, oxidations, acylations, aromatic substitutions, both electrophilic and nucleophilic, etherifications, esterification and saponification and the like. These manipulations are discussed in standard texts such as March Advanced Organic Chemistry (Wiley), Carey and Sundberg, Advanced Organic Chemistry (incorporated herein by reference in their entirety) and the like.

The skilled artisan will readily appreciate that certain reactions are best carried out when other functionality is masked or protected in the molecule, thus avoiding any undesirable side reactions and/or increasing the yield of the reaction. Often the skilled artisan utilizes protecting groups to accomplish such increased yields or to avoid the undesired reactions. These reactions are found in the literature and are also well within the scope of the skilled artisan. Examples of many of these manipulations can be found for example in T. Greene and P. Wuts Protecting Groups in Organic Synthesis, 4th Ed., John Wiley & Sons (2007), incorporated herein by reference in its entirety.

The following example schemes are provided for the guidance of the reader, and represent preferred methods for making the compounds exemplified herein. These methods are not limiting, and it will be apparent that other routes may be employed to prepare these compounds. Such methods specifically include solid phase based chemistries, including combinatorial chemistry. The skilled artisan is thoroughly equipped to prepare these compounds by those methods given the literature and this disclosure. The compound numberings used in the synthetic schemes depicted below are meant for those specific schemes only, and should not be construed as or confused with same numberings in other sections of the application.

Trademarks used herein are examples only and reflect illustrative materials used at the time of the invention. The skilled artisan will recognize that variations in lot, manufacturing processes, and the like, are expected. Hence the examples, and the trademarks used in them are non-limiting, and they are not intended to be limiting, but are merely an illustration of how a skilled artisan may choose to perform one or more of the embodiments of the invention.

The following abbreviations have the indicated meanings:
ACN or MeCN=acetonitrile
cod=cyclooctadiene
DCM=dichloromethane
DMF=N,N-dimethylformamide
DMAP=4-dimethylaminopyridine
dppf=1,1′-bis(diphenylphosphino)ferrocene
EDCI=N-(3-dimethylaminopropyl)-N′-ethylcarbodiimide hydrochloride
ESBL=extended-spectrum β-lactamase
EtOAc or EA=ethyl acetate
HATU=2-(7-aza-1H-benzotriazole-1-yl)-1,1,3,3-tetramethyluronium hexafluorophosphate
LDA=lithium diisopropylamide
MIC=minimum inhibitory concentration
NMR=nuclear magnetic resonance
PE=petroleum ether
RFC=radial flow chromatography
rt=room temperature
TBAF=tetrabutylammonium fluoride
TBSCl=tert-butyldimethylsilyl chloride
TBS=tert-butyldimethylsilyl
TES=triethylsilane
TFA=trifluoroacetic acid
TFAA=trifluoroacetic anhydride
THF=tetrahydrofuran
TIPS=triisopropylsilyl
TLC=thin layer chromatography
The following example schemes are provided for the guidance of the reader, and collectively represent an example method for making the compounds provided herein. Furthermore, other methods for preparing compounds described herein will be readily apparent to the person of ordinary skill in the art in light of the following reaction schemes and examples. Unless otherwise indicated, all variables are as defined above.

### Example 1

### 2-Hydroxy-4-(hydroxymethyl)-3.4-dihydro-1,2-benzoxaborinine-8-carboxylic Acid (Compound 1)

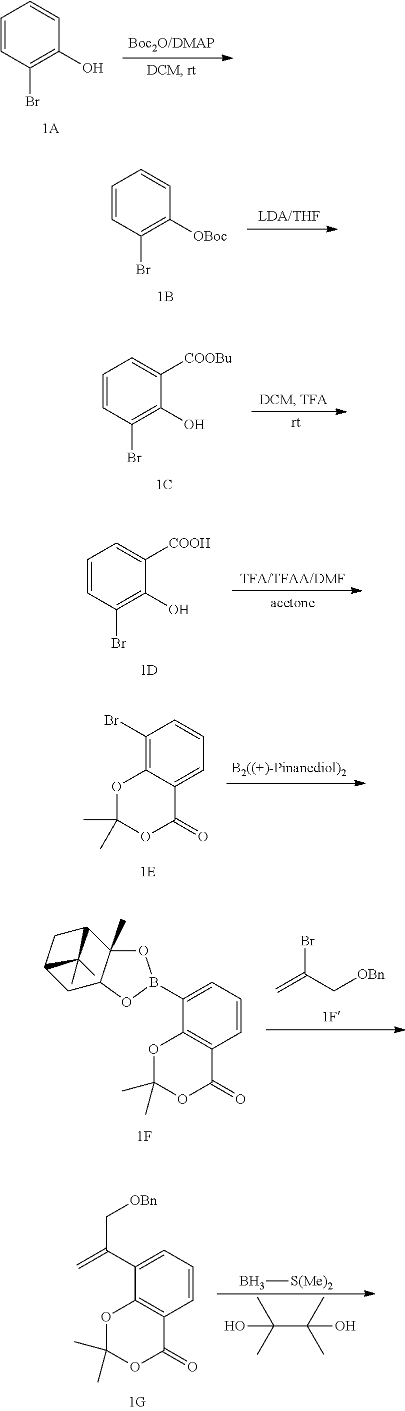

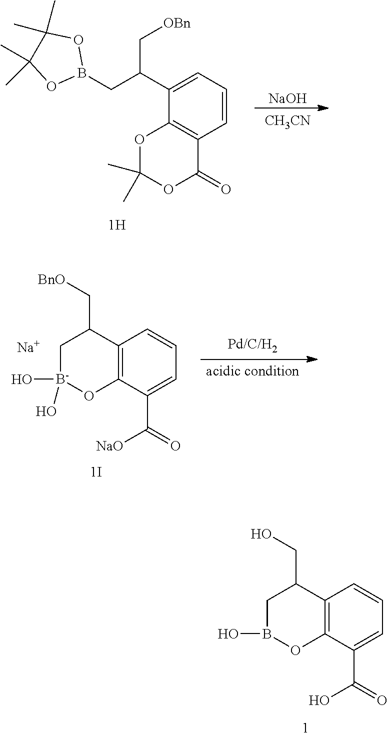

### Step 1: Synthesis of 1B

To a mixture of compound 1A (20 g, 116 mmol, 1.0 eq) and DMAP (4.2 g, 34 mmol, 0.3 eq) in DCM (200 mL) was added Boc₂O (37.8 g, 173 mmol, 1.5 eq) and the resulting solution was stirred at rt for 1 hour. The reaction was monitored by TLC. The mixture was concentrated under reduced pressure and the residue was purified by flash chromatography on silica gel (PE/EA=50:1 to 20:1) to give compound 1B (31 g, 98%) as light yellow oil.

### Step 2: Synthesis of 1C

To the solution of compound 1B (34 g, 125 mmol, 1.0 eq) in THF (350 mL) was added LDA (75 mL, 150 mmol, 1.2 eq) dropwise at −78° C. The resulting solution was slowly warmed up to rt and stirred for 16 hours. The reaction was monitored by TLC. The mixture was concentrated under reduced pressure and the residue was purified by flash chromatography on silica gel (PE/EA=50:1 to 20:1) to give compound 1C (21.8 g, 64%) as light yellow oil.

### Step 3: Synthesis of 1D

To the solution of compound 1C (21.8 g, 79.8 mmol, 1.0 eq) in DCM (110 mL) was added TFA (110 mL) at rt. After 16 hours at this temperature, the mixture was concentrated under reduced pressure and the residue was purified by flash chromatography on silica gel (PE/EA=50:1 to 10:1) to give compound 1D (13.9 g, 80%) as a white solid.

### Step 4: Synthesis of 1E

To the solution of compound 1D (14.7 g, 68 mmol, 1.0 eq) in TFA (95 mL) was added DMF (65 mL) at 0° C., followed by slow addition of acetone (50.6 mL) and TFAA (65 mL) at the same time. After 16 hours at 100° C. under nitrogen atmosphere, the mixture was concentrated under reduced pressure and the residue was purified by flash chromatography on silica gel (PE/EA=50:1 to 10:1) to give compound 1E (7.7 g, 44%) as a yellow solid.

### Step 5: Synthesis of 1F

To a mixture of compound 1E (5 g, 19.53 mmol, 1.0 eq) in dioxane (50 mL) was added B₂((+)-pinanediol)₂ (10.5 g, 29.30 mmol, 1.5 eq), PdCl₂(dppf) (797 mg, 0.98 mmol, 0.05 eq) and KOAc (3.8 g, 39.06 mmol, 2.0 eq). The mixture was stirred at 95° C. overnight under nitrogen atmosphere. The mixture was filtered and the filtrate was diluted with EA and water. The organic layer was washed with brine, dried over Na₂SO₄, concentrated in vacuum. The residue was purified by column chromatography on silica gel (PE/EA=1:0 to 10:1) to give compound 1F (2.0 g, 29%).

### Step 6: Synthesis of 1G

To a solution of compound 1F (1.0 g, 2.81 mmol, 1.0 eq) in THF (10 mL) was added 1F′ (1.3 g, 5.62 mmol, 2.0 eq), Pd(PPh₃)₄ (162 mg, 0.14 mmol, 0.05 eq) and 2 N Na₂CO₃ (7.0 mL, 14.0 mmol, 5 eq). The mixture was stirred at 80° C. overnight under nitrogen atmosphere. Then the mixture was diluted with EA washed with water and brine, dried over Na₂SO₄, concentrated in vacuum. The residue was purified by column chromatography on silica gel (PE/EA=1:0 to 10:1) to give compound 1G (715 mg, 78%).

### Step 7: Synthesis of 1H

To a solution of 2 M BH₃—S(Me)₂ (1.2 mL, 2.47 mmol, 2.0 eq) in dry THF (10 mL) at −15° C. under nitrogen atmosphere was added a solution of compound 1G (400 mg, 1.24 mmol, 1.0 eq) in dry THF (1 mL), slowly. The reaction mixture was stirred at rt for 2 h, quenched by water, and extracted with EA. The organic layer was washed with brine, dried over Na₂SO₄, and concentrated in vacuum. The residue was dissolved in dry THF (8 mL), and 2,3-Dimethylbutane-2,3-diol (292 mg, 2.47 mmol, 2.0 eq) was added. The reaction mixture was stirred at rt overnight. Then the reaction was filtered and the filtrate was concentrated. The residue was purified by column chromatography on silica gel (PE/EA=30:1 to 10:1) to give compound 1H (106 mg, 19%).

### Step 8: Synthesis of 1I

To a solution of compound 1H (120 mg, 0.266 mmol, 1.0 eq) in H₂O/ACN (1 mL/1 mL) was added 0.5 M NaOH (1 mL, 0.5 mmol, 1.8 eq) and the resulting mixture stirred at rt for 3 h. Then the mixture was purified by prep-HPLC (under neutral conditions) to give compound 1I (50 mg, 60%).

### Step 9: Synthesis of 1

To a solution of compound 1I (110 mg, 0.70 mmol, 1.0 eq) in MeOH (10 mL) was added Pd/C (11 mg, 10%, w/w). The resulting mixture was stirred at rt for 4 h under 1 atm of H₂. After filtration through a pad of Celite®, the filtrate was purified by prep-HPLC (under acidic conditions) to give compound 1 (20 mg, 26%).

### LC-MS: 221 [M−H]⁻

¹H NMR (400 MHz, CD₃OD) δ7.75-7.70 (m, 1H), 7.46-7.35 (m, 1H), 6.87-6.81 (dd, J=7.6, 8.0 Hz, 1H), 4.09 (d, J=6.8 Hz, 1H), 3.76-3.72 (m, 1H), 3.59-3.57 (m, 1H), 1.20-1.18 (m, 2H)

### Example 2

### Disodium Salt of 4-(benzyloxymethyl)-2-hydroxy-7-methoxy-3,4-dihydro-1,2-benzoxaborinine-8-carboxylic Acid (Compound 2)

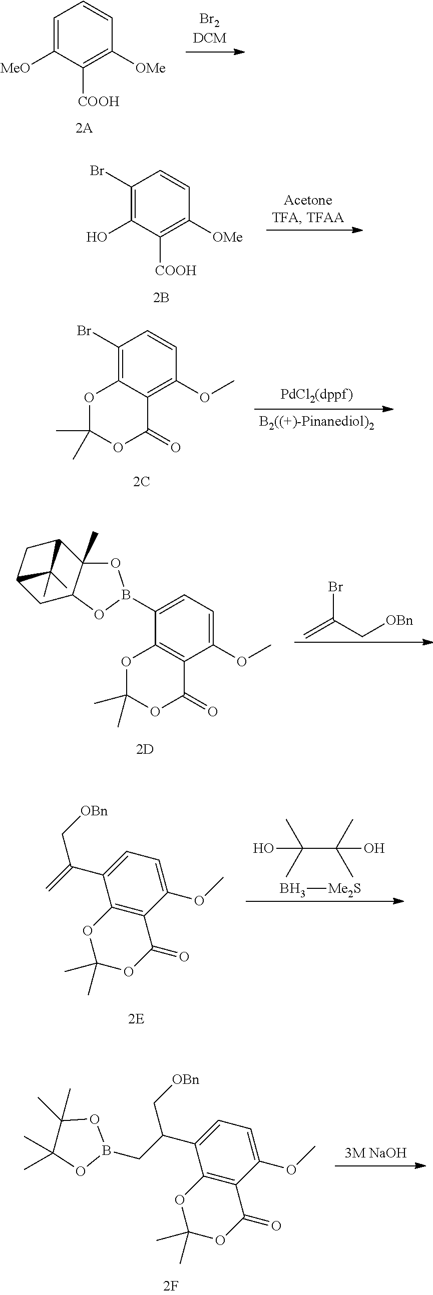

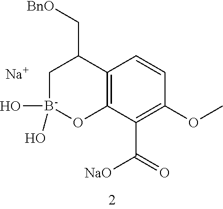

### Step 1: Synthesis of 2B

A solution of bromine (14.06 mL, 274 mmol, 1 eq.) in CH₂Cl₂ (20 mL) was added slowly over 8 h to a suspension of 2,6-dimethoxybenzoic acid (2A) (50 g, 274 mmol) in CH₂Cl₂ (200 mL). After stirring at rt overnight, the light orange slurry was heated and a portion of the solvent (methyl bromide, hydrogen bromide and CH₂Cl₂) was removed by distillation at atmospheric pressure (total volume distilled 100 mL). Ethanol (150 mL) was added and the remaining CH₂Cl₂ was distilled off at atmospheric pressure, slowly increasing the bath temperature to 90° C. Upon completion of the distillation (1 h), the heterogeneous mixture was cooled to rt. After stirring 1 h at rt, the slurry was cooled to 0° C. After stirring at 0° C. for 2 h, the solids were collected by filtration. The filtrate was recirculated to rinse the flask and stir bar. The solids were rinsed with ethanol at 0° C. (2×50 mL), air dried, then dried under high vacuum to give compound 2B as fine white needles (58.23 g, 85.9%).

### Step 2: Synthesis of 2C

A 10-ml syringe filled with trifluoroacetic anhydride (11.25 mL, 81 mmol, 2 eq) and a 20-ml syringe filled with acetone (17 mL, 232 mmol, 5.7 eq) simultaneously dispensed their contents via syringe pump over 24 hours into a clear solution of 2B (10 g, 40 mmol) in TFA (10 mL) at 70° C. After 1 hour, the starting material began to crystallize out. TFA (5 mL) was added, affording a clear solution. After another hour at 70° C. the solution became slightly heterogeneous. Upon completion of the addition, HPLC showed 89:11 product to starting material. After stirring at 70° C. overnight, the ratio was 92:8. The reaction mixture was cooled to rt, diluted with ethyl acetate (15 mL), filtered over Celite®, and the pad and flask were rinsed with ethyl acetate (2×10 mL). The clear black filtrate was concentrated to dryness. The solids were taken up in ethyl acetate (50 mL) and CH₂Cl₂ (10 mL, to improve solubility of the product) and washed twice with a saturated solution of NaHCO₃ (50 and 30 mL). The brown/black solution was concentrated to dryness. The residue was taken up in ethyl acetate (10 mL) and the mixture was heated to reflux. Heptane (3×10 mL) was added and the mixture was brought to reflux (after the last addition of heptane, the product started crystallizing). The heterogeneous mixture was refluxed for 15 min and was allowed to cool to rt. After stirring at rt for 2 hours and 0° C. for 2 hours, the solids were collected by filtration. The filtrate was recirculated to rinse the flask. The solids were rinsed with 3:1 heptane/ethyl acetate at 0° C. (2 ×10 mL), air dried, then dried under high vacuum to give compound 2C as a light tan powder (8.83 g, 76%).

### Step 3: Synthesis of 2D

To a mixture of compound 2C (10.0 g, 3.5 mmol, 1.0 eq) in dioxane (300 mL) was added B₂((+)-pinanediol)₂ (18.8 g, 52.5 mmol, 1.5 eq), PdCl₂(dppf) (2.8 g, 3.5 mmol, 0.1 eq) and KOAc (6.86 g, 70 mmol, 2.0 eq). The mixture was stirred at 96° C. overnight under nitrogen atmosphere. Then the mixture was filtered and the filtrate was and diluted with EA, washed with water and brine, dried over Na₂SO₄, and concentrated in vacuum. The residue was purified by column chromatography on silica gel (PE/EA=30:1 to 5:1) and triturated with PE/EA (10:1) to give compound 2D (7.9 g, 58%).

### Step 4: Synthesis of 2E

To a mixture of compound 2D (7.3 g, 19.06 mmol, 1.0 eq) in THF (120 mL) was added bromide 1F′ (5.6 g, 24.8 mmol, 1.3 eq), Pd(PPh₃)₄ (1.1 g, 0.95 mmol, 0.05 eq) and 2 N Na₂CO₃ (48 mL, 95 mmol, 5.0 eq). The mixture was stirred at 80° C. for 12 h under nitrogen atmosphere. The mixture was filtered and the filtrate was and extracted with EA and H₂O, and the organic layer was separated and washed with brine, dried over Na₂SO₄, and concentrated in vacuum. The residue was purified by column chromatography on silica gel (PE/EA=20:1 to 5:1) to give compound 2E (9.0 g, 100%, contained some pinanediol).

### Step 5: Synthesis of 2F

To a solution of 2 M BH₃—S(Me)₂ (32 mL, 63.56 mmol, 1.5 eq) in dry THF (300 mL) at −15° C. under nitrogen atmosphere was added a solution of compound 2E (15 g, 42.37 mmol, 1.0 eq) in dry THF (50 mL) slowly. The mixture was then moved away from the ice-bath and stirred for 2 h at rt. The mixture was quenched with water and extracted with EA. The organic layer was washed with brine, dried over Na₂SO₄, and concentrated in vacuum. The residue was dissolved in dry THF (150 mL), pinacol (7.5 g, 63.56 mmol, 1.5 eq) was added, and the resulting mixture stirred at rt overnight. The reaction was filtered and the filtrate was concentrated. The residue was purified by column chromatography on silica gel (PE/EA=30:0 to 5:1) to give compound 2F (8 g, 39%).

### Step 6: Synthesis of 2

To a solution of compound 2F (8 g, 16.59 mmol, 1.0 eq) in H₂O/ACN (7 mL/15 mL) was added 3 M NaOH (11.1 mL, 33.3 mmol, 2 eq), and the mixture was stirred at rt for 20 h. The mixture was purified by prep-HPLC to give compound 2 (6 g, 100%).

### LC-MS: 343 [M+H]⁺

¹H NMR (400 MHz, CD₃OD): δ7.41-7.18 (m, 5H), 7.04-6.93 (m, 1H), 6.29-6.16 (m, 1H), 4.51 (m, 2H), 3.75-3.68 (m, 3H), 3.67-3.59 (m, 2H), 2.98-2.85 (m, 1H), 0.61-0.50 (m, 1H), 0.41-0.29 (m, 1H).

### Example 3

### 2-Hydroxy-4-(hydroxymethyl)-7-methoxy-3,4-dihydro-1,2-benzoxaborinine-8-carboxylic Acid (Compound 3)

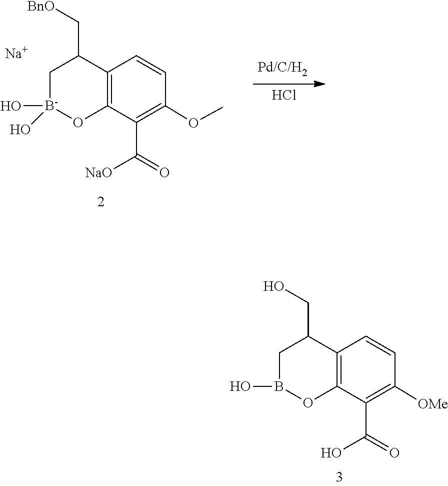

To a solution of compound 2 (2.0 g, 5.85 mmol, 1.0 eq) in methanol (20 mL) was added 1 N HCl (to adjust the solution to pH 5-6) and Pd/C (200 mg, 10% W/W). The mixture was stirred under H₂ at rt for 16 h, filtered through Celite® and purified by prep-HPLC (under acidic conditions) to give compound 3 (310 mg, 21%).

### LC-MS: 253 [M+H]⁺

¹H NMR (400 MHz, CD₃OD) δ7.21-7.20 (m, 1H), 6.67-6.65 (d, J=8.4 Hz, 1H), 3.84 (s, 3H), 3.58-3.57 (m, 2H), 3.02-2.85 (m, 1 H), 1.15-1.13 (m, 2H).

### Example 4

(4R)-2-hydroxy-4-(hydroxymethyl)-7-methoxy-3,4-dihydro-1,2-benzoxaborinine-8-carboxylic Acid and (4S)-2-hydroxy-4-(hydroxymethyl)-7-methoxy-3,4-dihydro-1,2-benzoxaborinine-8-carboxylic Acid (Compounds 4 and 5)

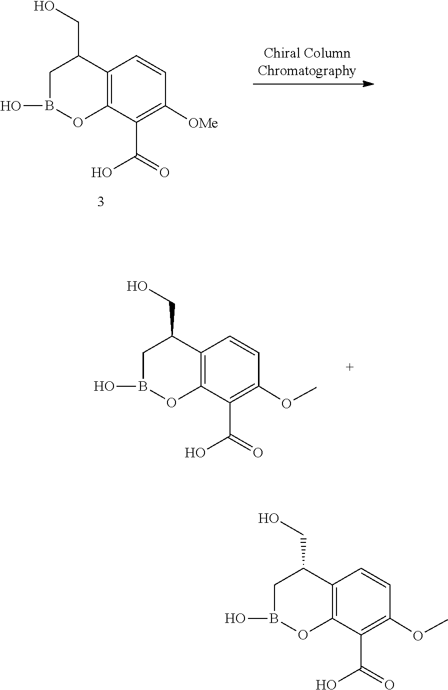 [SMILES: COc1ccc2c(c1C(=O)O)OB(O)CC2CO ; COc1ccc2c(c1C(=O)O)OB(O)C[C@H]2CO ; COc1ccc2c(c1C(=O)O)OB(O)C[C@@H]2CO]

2-Hydroxy-4-(hydroxymethyl)-7-methoxy-3,4-dihydro-1,2-benzoxaborinine-8-carboxylic acid (Compound 3) (1.03 g) was separated by chiral column (Superchiral S-AD, Hexane/EtOH/MeOH/formic acid=60/13/27/0.01, v/v/v/v) to give Compound 4 344 mg, 33%) and Compound 5, 410 mg, 39%. Compound 4 and Compound 5 are depicted as the stereoisomers in the scheme above, although absolute stereochemistry of the individual isomers is yet to be determined.

### Compound 4

### LC-MS: 251 [M−H]⁻

¹H NMR (400 MHz, CD₃OD) δ7.21-7.20 (m, 1H), 6.67-6.65 (d, J=8.4 Hz, 1H), 3.84 (s, 3H), 3.58-3.57 (m, 2H), 3.02-2.85 (m, 1 H), 1.15-1.13 (m, 2H).

### Compound 5:

### LC-MS: 251 [M−H]⁻

¹H NMR (400 MHz, CD₃OD) δ7.21-7.20 (m, 1H), 6.67-6.65 (d, J=8.4 Hz, 1H), 3.84 (s, 3H), 3.58-3.57 (m, 2H), 3.02-2.85 (m, 1 H), 1.15-1.13 (m, 2H).

### Example 5

### 7-fluoro-2-hydroxy-4-(hydroxymethyl)-3,4-dihydro-1,2-benzoxaborinine-8-carboxylic Acid (Compound 6)

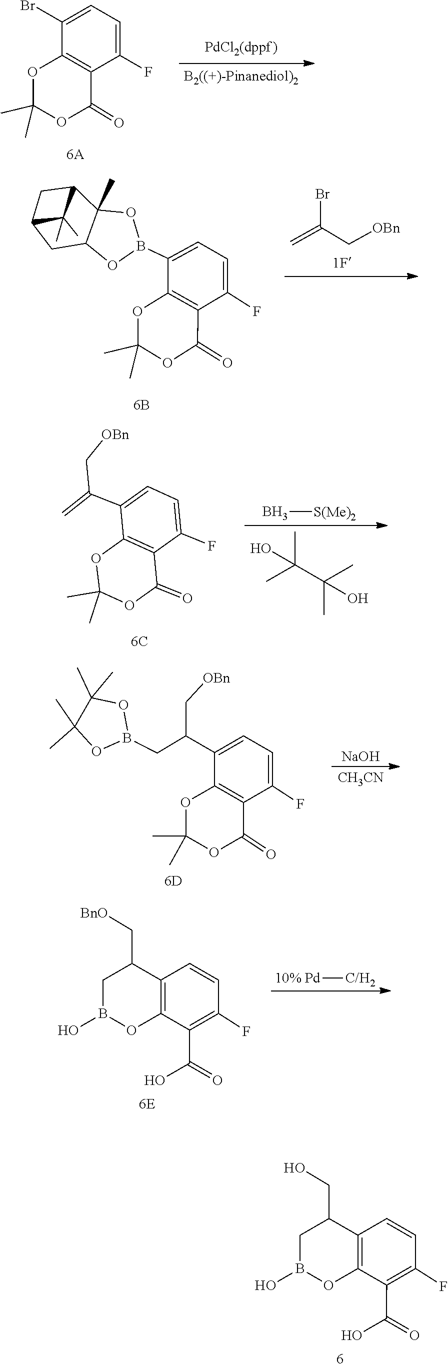

### Step 1: Synthesis of Compound 6A

Compound 6A was prepared from a Boc-t-Butyl ester intermediate (previously disclosed in WO 2015/179308) by TFA deprotection (as described in step 3 of Example 1) followed by isopropylidene protection (as described in step 2 of Example 2).

### Step 2: Synthesis of Compound 6B

To a mixture of bromide compound 6A (20.0 g, 72.99 mmol, 1.0 eq) in dioxane (200 mL) was added B₂(+)-Pinanediol)₂ (39.2 g, 109.5 mmol, 1.5 eq), PdCl₂(dppf) (3.0 g, 3.65 mmol, 0.05 eq) and KOAc (14.3 g, 145.9 mmol, 2.0 eq). The mixture was stirred at 95° C. overnight under nitrogen atmosphere. The mixture was filtered and the filtrate diluted with EtOAc and washed with water, brine, dried over Na₂SO₄, and concentrated in vacuum. The residue was purified by column chromatography on silica gel (PE/EA=1:0 to 10:1) to give compound 6B (13.5 g, 49%).

### Step 3: Synthesis of Compound 6C

To a mixture of compound 6B (13.5 g, 36.1 mmol, 1.0 eq) in THF (90 mL) was added 1F′ (12.2 g, 54.14 mmol, 1.5 eq), Pd(PPh₃)₄ (2.1 g, 1.80 mmol, 0.05 eq) and 2 N Na₂CO₃ (90 mL, 180.5 mmol, 5.0 eq). The mixture was stirred at 80° C. overnight under nitrogen atmosphere. Then the mixture was filtered through a pad of Celite®, and water was added. The mixture was extracted with EA. The combined organic layers were washed with water, brine, dried over Na₂SO₄, and concentrated in vacuum. The residue was purified by column chromatography on silica gel (PE/EA=50:1 to 10:1) to give compound 6C (9.8 g, 79%).

### Step 4: Synthesis of Compound 6D

To a solution of 2 M BH₃—S(Me)₂ (1.5 mL, 2.92 mmol, 2.0 eq) in dry THF (10 mL) at −15° C. under nitrogen atmosphere was added a solution of compound 6C (500 mg, 1.462 mmol, 1.0 eq) in dry THF (2 mL) slowly. The reaction mixture was stirred at rt for 2 h, quenched with water, and extracted with EA. The organic layer was washed with brine, dried over Na₂SO₄, and concentrated in vacuum. The residue was dissolved in dry THF (8 mL). To the resulting mixture was added 2,3-dimethylbutane-2,3-diol (345 mg, 2.92 mmol, 2.0 eq). The reaction was stirred at rt overnight. Then the reaction was filtered and the filtrate was concentrated. The residue was purified by column chromatography on silica gel (PE/EA=30:0 to 10:1) to give compound 6D (188 mg, 27%).

### Step 5: Synthesis of Compound 6E

To a solution of compound 6D (188 mg, 0.4 mmol, 1.0 eq) in H₂O/ACN (1 mL/1 mL) was added 3 M NaOH (0.26 mL, 0.8 mmol, 2.0 eq) and the resulting mixture was stirred at rt overnight. The crude product 6E was used for next step directly.

### Step 6: Synthesis of Compound 6

To the crude of 6E was added Pd/C (18 mg, 10% w/w). The mixture was stirred at rt overnight under H₂. After filtration, the mixture was purified by prep-HPLC (under neutral conditions) to give 8 mg of compound 6.

LC-MS: 282 [M+MeCN+H]⁺; 239 [M−H]⁻
¹H NMR (400 MHz, CD₃OD) δ7.03 (m, 1H), 6.40-6.38 (m, 1H), 3.87-3.83 (m, 1H), 3.71-3.68 (m, 1H), 3.04-3.03 (m, 1H), 0.71 (m, 2H)

### Example 6

(4R)-7-fluoro-2-hydroxy-4-(hydroxymethyl)-3,4-dihydro-1,2-benzoxaborinine-8-carboxylic acid and (4S)-7-fluoro-2-hydroxy-4-(hydroxymethyl)-3,4-dihydro-1,2-benzoxaborinine-8-carboxylic Acid (Compounds 7 and 8)

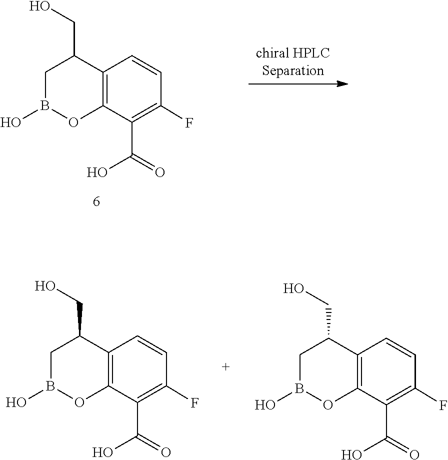 [SMILES: O=C(O)c1c(F)ccc2c1OB(O)CC2CO ; O=C(O)c1c(F)ccc2c1OB(O)C[C@H]2CO ; O=C(O)c1c(F)ccc2c1OB(O)C[C@@H]2CO]

7-Fluoro-2-hydroxy-4-(hydroxymethyl)-3,4-dihydro-1,2-benzoxaborinine-8-carboxylic acid (Compound 6) was separated by HPLC with a chiral column (Superchiral S-AD, Hexane/EtOH/MeOH/TFA=90/3.3/6.7/0.05 (v/v/v)) to give Compound 7 (40.8 mg, 16%) and Compound 8 (33.0 mg, 13%). Compound 7 and Compound 8 are depicted as the stereoisomers in the scheme above, although absolute stereochemistry of the individual isomers is yet to be determined.

### Compound 7

### LC-MS: 239 [M−H]⁻

¹H NMR (400 MHz, CD₃OD) δ7.32-7.29 (m, 1H), 6.71-6.67 (m, 1H), 3.97-3.79 (m, 1H), 3.76-3.54 (m, 2H), 1.18-1.13 (m, 2H).

### Compound 8

### LC-MS: 239[M−H]⁻

¹H NMR (400 MHz, CD₃OD) δ7.32-7.29 (m, 1H), 6.71-6.67 (m, 1H), 3.97-3.79 (m, 1H), 3.76-3.54 (m, 2H), 1.18-1.13 (m, 2H).

### Example 7

### 2-Hydroxy-4-(hydroxymethyl)-3,4-dihydrooxaborinino[6,5-c]pyridine-8-carboxylic Acid (Compound 9)

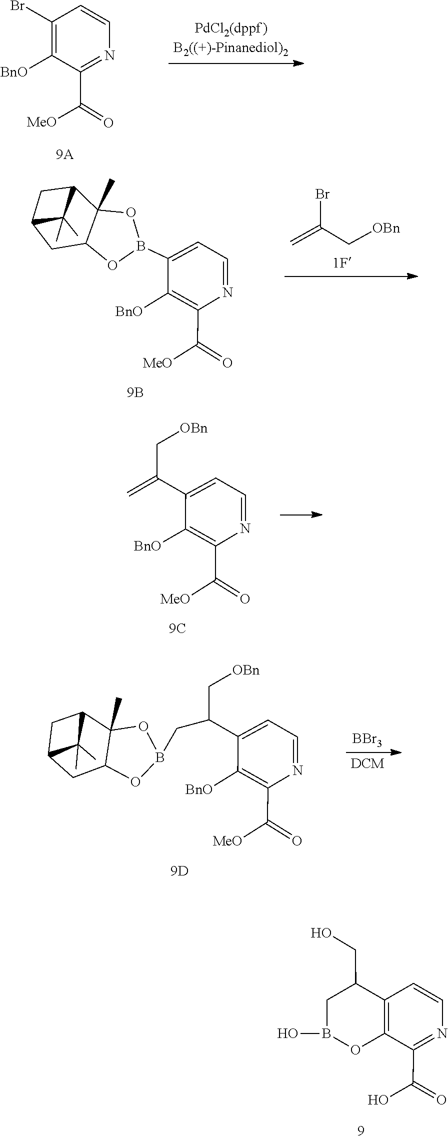

### Step 1: Synthesis of Compound 9B

To a mixture of compound 9A (Tetrahedron, 2011, 67, 8757-8762) (1.0 g, 3.12 mmol, 1.0 eq) in dioxane (10 mL) was added B2((+)-pinanediol)₂ (1.67 g, 4.67 mmol, 1.5 eq), PdCl₂(dppf) (255 mg, 0.31 mmol, 0.1 eq) and KOAc (916 mg, 9.35 mmol, 3.0 eq). The mixture was stirred at 55° C. overnight under nitrogen atmosphere. The mixture was filtered and the filtrate was extracted with EA and H₂O, and the organic layer was separated, washed with brine, dried over Na₂SO₄, and concentrated in vacuum. The residue was purified by column chromatography on silica gel (PE/EA/DCM=30:1:0 to 5:1:1) to give compound 9B (1.1 g, 84%).

### Step 2: Synthesis of Compound 9C

To a mixture of compound 9B (1.1 g, 2.61 mmol, 1.0 eq) in THF (90 mL) was added 1F′ (1.18 g, 5.22 mmol, 2.0 eq), Pd(PPh₃)₄ (151 mg, 0.13 mmol, 0.05 eq), and 2 N Na₂CO₃ (6.5 mL, 13.0 mmol, 5 eq). The mixture was stirred at 80° C. overnight under nitrogen atmosphere. Then the mixture was filtered and the filtrate was washed with ethyl acetate. The organic layer was washed with water and brine, dried over Na₂SO₄, and concentrated in vacuum. The crude product was purified by column chromatography on silica gel (PE/EA=20:1 to 5:1) to give compound 9C (978 mg, 96%).

### Step 3: Synthesis of Compound 9D

To a mixture of compound 9C (350 mg, 0.90 mmol, 1.0 eq) in methanol (2 mL) was added B2((+)-pinanediol)₂ (370 mg, 1.03 mmol, 1.15 eq), Cu₂O (10 mg, 0.072 mmol, 0.08 eq), PPh₃ (26 mg, 0.099 mmol, 0.11 eq), and KH₂PO₄ (188 mg, 1.079 mmol, 1.2 eq). The mixture was stirred at 40° C. for 2.5 h under nitrogen atmosphere. Then the mixture was filtered and the filtrate was washed with EA, and the organic layer was washed with brine, dried over Na₂SO₄, and concentrated in vacuum. The residue was purified by column chromatography on silica gel (PE/EA=20:1 to 2:1) to give compound 9D (350 mg, 68%).

### Step 4: Synthesis of Compound 9

To a solution of compound 9D (197 mg, 0.411 mmol, 1.0 eq) in DCM (1.5 mL) was added 1 M BBr₃ (1.3 mL, 1.30 mmol, 5 eq), the mixture was stirred at rt for 1 h, and then the mixture was purified by prep-HPLC (under acidic conditions) to give compound 9 (5.5 mg).

### LC-MS: 224 [M+H]⁺

¹H NMR (400 MHz, D₂O) δ8.20-8.19 (d, J=5.6 Hz, 1H), 7.90-7.89 (d, J=5.6 Hz, 1H), 3.91-3.89 (m, 1H), 3.85-3.81 (m, 1H), 3.73-3.70 (m, 1H), 1.25-1.20 (m, 2H).

### Example 8

### Disodium Salt 4-(benzyloxymethyl)-2-hydroxy-3,4-dihydrooxaborinino[6,5-c]pyridine-8-carboxylic Acid (Compound 10)

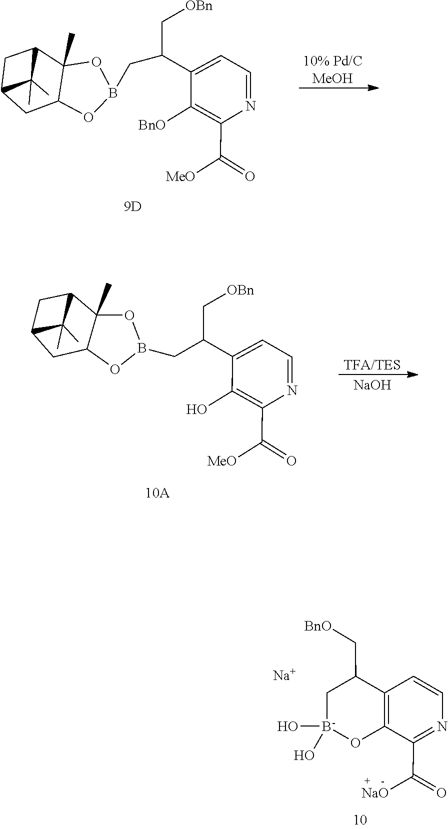

Step 1: Synthesis of compound 10A

A mixture of compound 9D (130 mg, 0.228 mmol, 1.0 eq) and Pd/C (13 mg, 10% w/w) in methanol (2 mL) was stirred at rt for 2 days under H₂. The resulting mixture was filtered and purified by prep-TLC to give compound 10A (70 mg, 64%).

### Step 2: Synthesis of Compound 10

To a solution of compound 10A (30 mg, 0.063 mmol, 1.0 eq) in H₂O/ACN (0.5 mL/0.5 mL) was added TFA (0.01 mL, 0.125 mmol, 2 eq), TES (0.05 mL), and i-BuB(OH)₂ (13 mg, 0.125 mmol, 2 eq). The mixture was stirred at 25° C. overnight, and adjusted to pH 10-11 with 0.5 N NaOH (0.25 mL). The resulting mixture was stirred at rt for 2 h, and purified by prep-HPLC (under neutral conditions) to give compound 10 (3.4 mg).

### LC-MS: 314 [M+H]⁺

¹H NMR (400 MHz, CD₃OD) δ7.73-7.71 (m, 1H), 7.33-7.20 (m, 6H), 4.56-4.53 (m, 2H), 3.70-3.69 (d, J=6.0 Hz, 2H), 3.05-3.04 (m, 1H), 0.68-0.64 (m, 1H), 0.39-0.35 (m, 1H)

### Example 9

### 2-Hydroxy-7-methoxy-4-(methoxymethyl)-3,4-dihydro-1,2-benzoxaborinine-8-carboxylic Acid (Compound 11)

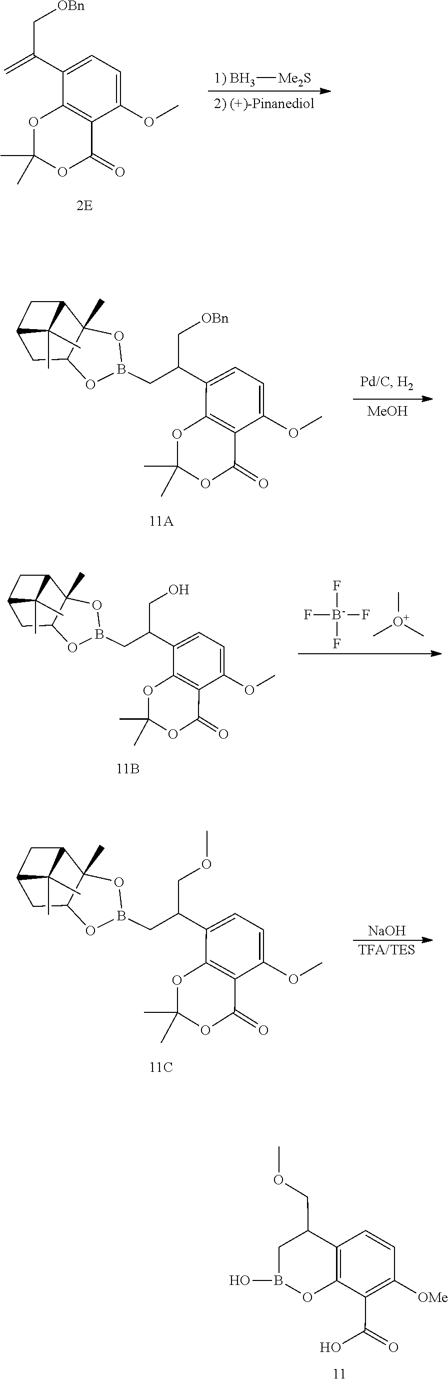

### Step 1: Synthesis of Compound 11A

To a solution of BH₃—S(Me)₂ (28.2 mL, 56.4 mmol, 2.0 eq) in dry THF (60 mL) at −15° C. was added compound 2E (10 g, 28.2 mmol, 1 eq). The mixture was warmed to rt and stirred for 3.5 h. TLC showed no compound 2E left. The mixture was diluted with water (30 mL) and extracted with EA (2×60 mL). The organic phase was dried over Na₂SO₄ and concentrated to give a residue, which was used for next step without further purification.

The residue prepared above was dissolved with dry THF (60 mL). (+)-Pinanediol (9.6 g, 56.4 mmol, 2.0 eq) was added into the solution. The mixture was stirred overnight at rt, and concentrated. The residue was purified by flash column chromatography (PE/EA=5:1) to give compound 11A (11 g, impure).

### Step 2: Synthesis of Compound 11B

To a solution of crude compound 11A (5.3 g) in methanol (53 mL) was added 10% Pd/C (530 mg, 10% w/w) at rt. The mixture was stirred under hydrogen atmosphere (balloon) overnight. The resulting mixture was filtered and the filtrate was concentrated. The residue was purified by flash column chromatography (PE/EA=2:1) to give compound 11B (1.2 g, 20%, 2 steps).

### Step 3: Synthesis of Compound 11C

To a mixture of compound 11B (250 mg, 0.56 mmol, 1.0 eq) in dry DCM (5 mL) was added trimethyloxonium tetrafluoroborate (167 mg, 1.13 mmol, 2.0 eq) and Cs₂CO₃ (183.5 mg, 0.563 mmol, 1.0 eq). The mixture was stirred at 25° C. overnight under nitrogen atmosphere. The resulting mixture was diluted with water and extracted with DCM. The DCM layer was washed with brine, dried over sodium sulfate, and concentrated in vacuo. The crude residue was purified by column chromatography on silica gel to give compound 11C (161 mg, 62%).

### Step 2: Synthesis of Compound 11

To a solution of compound 11C (155 mg, 0.34 mmol, 1.0 eq) in H₂O/ACN (1.5 mL/1.5 mL) was added 3 N NaOH (0.34 mL, 1.02 mmol, 3 eq). The mixture was stirred at 25° C. overnight. To the mixture were added TFA (0.06 mL), TES (0.06 mL) and i-BuB(OH)₂ (68 mg, 0.68 mmol, 2 eq). The resulting mixture was stirred at 25° C. for 1 h, and purified by prep-HPLC (under acidic conditions) to give compound 11 (57 mg, 63%).

### LC-MS: 267 [M+H]⁺

¹H NMR (400 MHz, CD₃OD) δ7.19 (d, J=8.0 Hz, 1H), 6.67 (d, J=8.8 Hz, 1H), 3.82 (s, 3H), 3.42-3.30 (m, 2 H), 3.25 (s, 3 H), 3.07-3.05 (m, 1H), 1.15-1.05 (m, 2H).

### Example 10

### 2-Hydroxy-4-(2-hydroxyethyl)-7-methoxy-3,4-dihydro-1,2-benzoxaborinine-8-carboxylic Acid Compound 12)

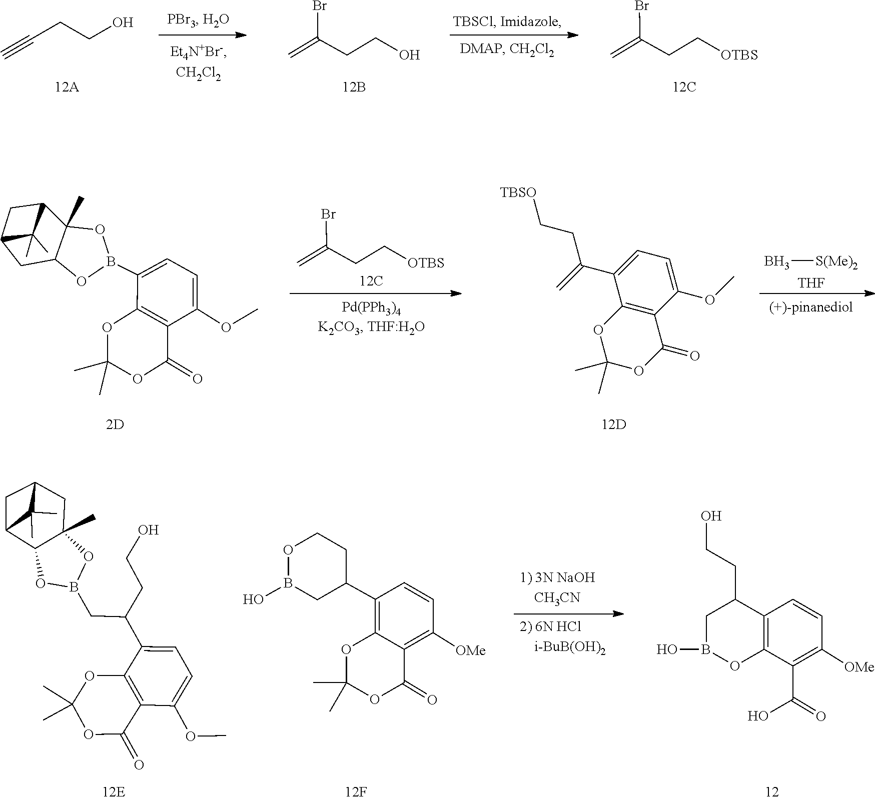

### Step 1: Synthesis of Compound 12B

Gaseous HBr was produced by adding PBr₃ (10.5 mL, 110 mmol) dropwise to water (6.0 mL, 330 mmol) at room temperature. The HBr gas produced was bubbled into a solution of Et₄N⁺Br— (97.0 g, 0.3 mol, 1.2 eq) in DCM (300 mL) at 0° C. To the HBr solution was added 3-butyn-1-ol (12A, 19.0 mL, 0.25 mol, 1.0 eq) and the solution was heated at 40° C. for 5 hrs. Removal of the solvent, followed by distillation, afforded 3-bromo-3-buten-1-ol, 12B (27.9 g, 73%).

### Step 2: Synthesis of Compound 12C

To a solution of 3-bromo-3-buten-1-ol (12B) (13.0 g, 86.1 mmol, 1.0 eq) in DCM (250 mL) at room temperature was added imidazole (7.74 g, 114 mmol, 1.3 eq), DMAP (2.15 g, 18.0 mmol, 0.2 eq), and TBSCl (14.5 g, 96.2 mmol, 1.1 eq). The reaction was stirred at room temperature for 3 hrs, filtered, washed with brine, dried over MgSO₄, concentrated, and purified via FCC (SiO₂, hexanes) to afford compound 12C (16.8 g, 73%).

### Step 3: Synthesis of Compound 12D

To a solution of 2D (503 mg, 1.30 mmol, 1.0 eq) in THF (3.6 mL) was added alkene 12C (688 mg, 2.60 mmol, 2.0 eq), followed by a 2 M solution of K₂CO₃ (1.6 mL, 3.2 mmol, 2.5 eq). After bubbling the solution with N₂ for 10 min, Pd(PPh₃)₄ (147 mg, 0.13 mmol, 0.1 eq) was added and the reaction mixture was heated to 70° C. for 16 hrs. The reaction was quenched with NaHCO₃ (saturated aq), extracted with EtOAc (2×), dried over Na₂SO₄, concentrated, and purified via FCC (SiO₂, 10% EtOAc/hexanes) to afford compound 12D (360 mg, 70%) as an orange oil.

### Step 4: Synthesis of Compounds 12E and 12F

To a solution of 12D (274 mg, 0.70 mmol, 1.0 eq) in THF (14 mL) was added BH₃—S(Me)₂ (1.2 eq, 0.42 mL, 2M in THF, 0.84 mmol, 1.2 eq) at 0° C. The reaction was allowed to warm up to room temperature slowly and stirred for 2 hrs. (+)-Pinanediol (291 mg, 1.7 mmol, 2.4 eq) was added and the reaction was stirred at room temperature overnight. The reaction mixture was quenched with H₂O, extracted with EtOAc (3×), dried over Na₂SO₄, and concentrated. The residue was purified with FCC (SiO₂, 40% EtOAc/hexanes), affording a mixture of 12E and 12F (109 mg), which were used for next step without further purification.

### Step 5: Synthesis of Compound 12

The mixture of compounds 12E and 12F (109 mg) was dissolved in acetonitrile (3.0 mL), and a 3 M solution of NaOH (1.2 mL) was added at room temperature. The reaction mixture was allowed to stir for 24 hrs. The resulting solution was adjusted to pH 2-3 using 6 N HCl. i-Bu(OH)₂ (53 mg) was added. The reaction was stirred overnight, and purified by prep-HPLC to afford compound 12 (7.5 mg, 4%) as white fluffy solid.

### LC-MS: 267.2 [M+1]⁺

¹H NMR (300 MHz, CD₃OD) δ7.28 (d, 1H), 6.80 (d, 1H), 4.05 (m, 2H), 3.92 (s, 3H), 3.30-3.18 (m, 1H), 1.90-1.68 (m, 2H), 1.20 (dd, 1H), 0.90 (dd, 1H).

### Example 11

### 2-Hydroxy-4-(hydroxymethyl)-7-methoxy-3,4-dihydro-1,2-benzoxaborinine-8-carboxylic Acid (Compound 3)

Compound 3 (described in Example 3) was also prepared using the following alternative synthetic sequence.

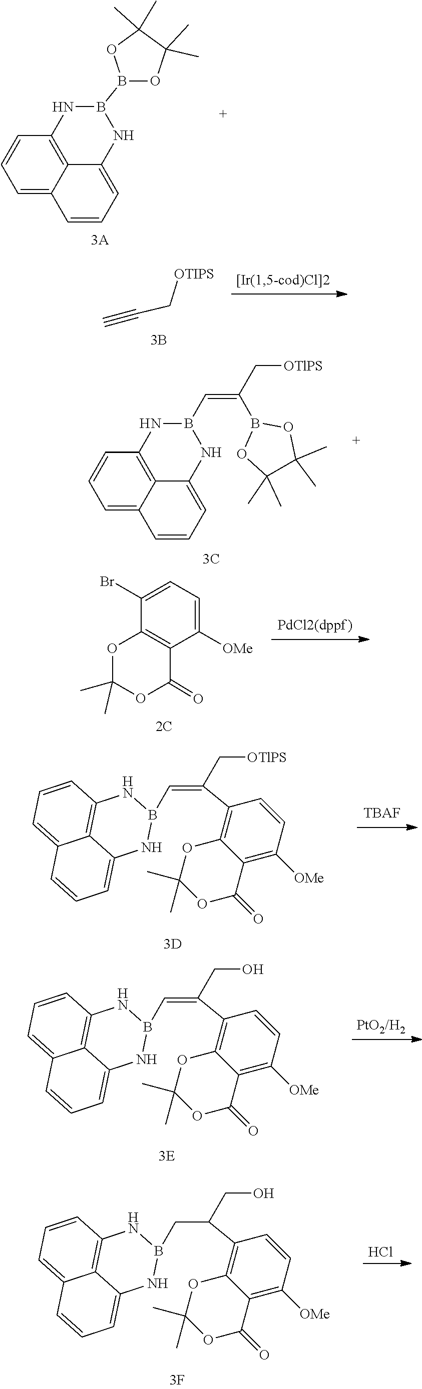

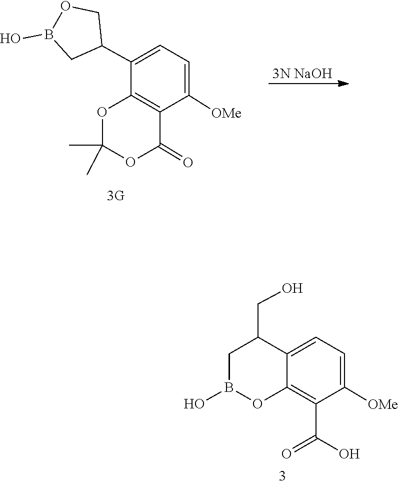 [SMILES: COc1ccc(C2COB(O)C2)c2c1C(=O)OC(C)(C)O2 ; "3N NaOH" ; COc1ccc2c(c1C(=O)O)OB(O)CC2CO]

### Step 1: Synthesis of Compound 3C

A solution of compound 3A (J. Org. Chem., 2016, 81, 4269-4279) (26 g, 0.123 mol, 1.5 eq), triisopropylsilyl propargyl ether (3B) (24 g, 0.082 mol, 1.0 eq), and Bis(1,5-cyclooctadiene)diiridium(I) dichloride (0.826 g, 1.23 mmol, 0.015 eq) in toluene (260 mL) was heated to 80° C. and stirred overnight under N₂. The resulting mixture was cooled to rt and filtered. The filtrate was concentrated and the residue was purified by flash column chromatography (PE/EA=5:1) to give compound 3C (35.7 g, 86%).

### Step 2: Synthesis of Compound 3D

To a solution of compound 3C (5.0 g, 9.881 mmol, 1.1 eq) in THF (50 mL) was added compound 2C (2.57 g, 8.983 mmol, 1.0 eq), PdCl₂(dppf) (368 mg, 0.494 mmol, 0.05 eq), water (1.625 g, 98.81 mmol, 10 eq), and K₃PO₄·3H₂O (7.175 g, 29.644 mmol, 3.0 eq). The mixture was stirred at 80° C. overnight under nitrogen atmosphere. The mixture was filtered and the filtrate was diluted with EA and H₂O. The layers were separated and the organic layer was washed with brine, dried over Na₂SO₄, and concentrated in vacuum. The residue was purified by column chromatography on silica gel to give compound 3D (4.79 g, 82%).

### Step 3: Synthesis of Compound 3E

To a solution of compound 3D (34 g, 58.0 mmol, 1.0 eq) in THF (300 mL) at rt was added TBAF (18.1 g, 69.6 mmol, 1.2 eq). The mixture was stirred for 3 h at rt and TLC showed no compound 3D left. The resulting mixture was diluted with water (150 mL) and extracted with EA (2×300 mL). The organic phase was dried over Na₂SO₄ and concentrated. The residue was purified with flash column chromatography (PE/EA=1:1) to give compound 3E (16.1 g, 81%).

### Step 4: Synthesis of Compound 3F

To a solution of compound 3E (16 g, 37.21 mmol) in methanol (160 mL) was added PtO₂ (1.6 g, 10% w/w) under N₂, then replaced with hydrogen. The mixture was stirred at rt for 6 h. The resulting mixture was filtered and the filtrate was concentrated. The residue was purified by silica gel column chromatography (PE/EA=1:1) to give compound 3F (13.6 g, 87%).

### Step 5: Synthesis of Compound 3G

To a solution of compound 3F (13.4 g, 31.02 mmol, 1.0 eq) in THF (150 mL) at rt was added 3 N aq. HCl (51.7 mL, 155.1 mmol, 5.0 eq). The mixture was stirred overnight and LC-MS showed 10% compound 3F remained. Then additional 3 N HCl (20.7 mL, 61.02 mmol, 2.0 eq) was added. The mixture was stirred at rt for 3 h, at which point LC-MS showed complete consumption of the starting material. Water (75 mL) was added to the reaction mixture, and the resulting solution was extracted with EA (2×75 mL). The organic phase was washed with water (75 mL), brine (75 mL), dried over sodium sulfate, and concentrated. The crude was purified by silica gel column chromatography (PE/EA=1:1) to give compound 3G (6.7 g, 75%).

### Step 6: Synthesis of Compound 3

To a solution of compound 3G (7.0 g, 23.9 mmol, 1.0 eq) in H₂O/CH₃CN (35 mL/35 mL) at rt was added 3 N NaOH (16 mL, 47.8 mmol, 2.0 eq). The mixture was stirred at rt for 1.5 h, and TLC showed no 3G was left. Then 3 N HCl was added to adjust the solution to pH ˜2. The resulting solution was stirred at rt overnight, and lyophilized. The solid was dissolved in water (20 mL) and extracted with EA (5×20 mL). The organic layers were dried and concentrated to give crude compound 3 (5.4 g). The compound was further purified by prep-HPLC (under acidic conditions) to give pure compound 3.

### LC-MS: 253 [M+H]⁺

¹H NMR (400 MHz, CD₃OD) δ7.08-7.06 (d, J=8.4 Hz, 1H), 6.43-6.41 (d, J=8.0 Hz, 1H), 3.81-3.78 (m, 1H), 3.77-3.74 (m, 3H), 3.67-3.63 (m, 1H), 2.96 (m, 1H), 0.78-0.76 (m, 2H).

### Example 12

### Disodium Salt of 4-(formamidomethyl)-2-hydroxy-7-methoxy-3,4-dihydro-1,2-benzoxaborinine-8-carboxylic Acid (Compound 13)

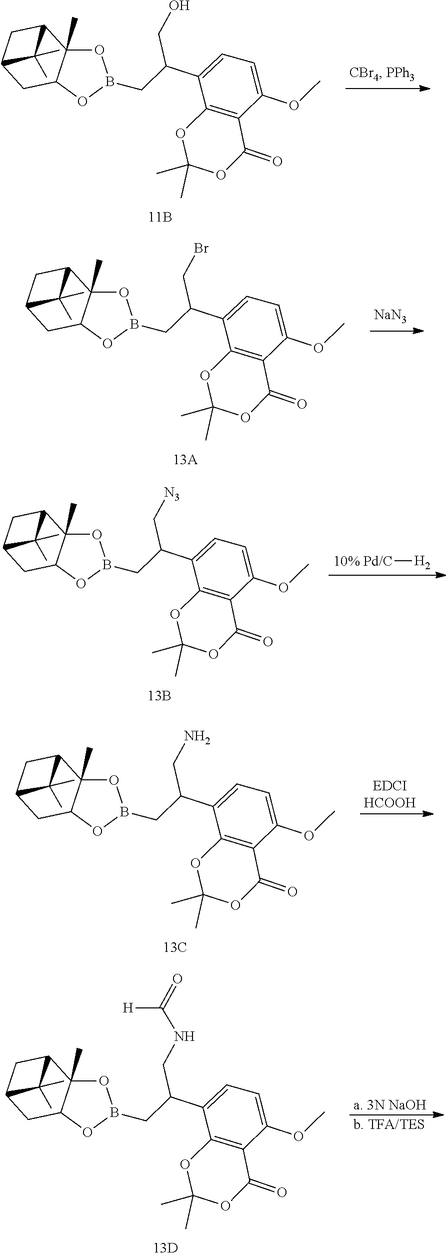

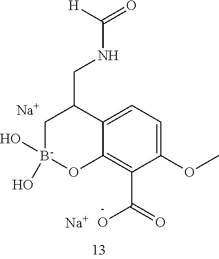 [SMILES: COc1ccc2c(c1C(=O)O)O[B-](O)(O)CC2CNC=O ; [Na+] ; [Na+]]

### Step 1: Synthesis of Compound 13A

To a mixture of compound 11B (1.5 g, 3.38 mmol, 1.0 eq) in DCM (10 mL) was added CBr₄ (1.68 g, 5.07 mmol, 1.5 eq) and PPh₃ (1.33 g, 5.07 mmol, 1.5 eq). The mixture was stirred at rt for 2 h under nitrogen atmosphere. Then the mixture was concentrated in vacuum. The residue was purified by column chromatography on silica gel (PE/EA=30:1 to 5:1) to give compound 13A (1.1 g, 64%).

### Step 2: Synthesis of Compound 13B

To a solution of compound 13A (280 mg, 0.55 mmol, 1.0 eq) in DMF (8 mL) was added NaN₃ (108 mg, 1.66 mmol, 3.0 eq). The mixture was stirred at 48° C. for 19 h under nitrogen atmosphere. Then the mixture was filtered and the filtrate was diluted with EA. The organic layer was washed water and with brine, dried over Na₂SO₄, and concentrated in vacuum to give crude compound 13B (250 mg, 96%), which was used for next step without further purification.

### Step 3: Synthesis of Compound 13C

To a mixture of compound 13B (250 mg, 0.533 mmol, 1.0 eq) in MeOH (30 mL) was added Pd/C (25 mg, 10% w/w) under hydrogen atmosphere. The mixture was stirred at rt for 2 days. Then the mixture was filtered and the filtrate was concentrated in vacuum to give crude compound 13C (220 mg, 93%), which was used for next step without further purification.

### Step 4: Synthesis of Compound 13D

To a mixture of compound 13C (500 mg, 1.13 mmol, 1.0 eq) in DCM (10 mL) was added EDCI (433 mg, 2.26 mmol, 2.0 eq), DMAP (14 mg, 0.113 mmol, 0.1 eq), and formic acid (104 mg, 2.26 mmol, 2.0 eq) under N₂. The mixture was stirred at rt for 4 h. Then the resulting mixture was purified by prep-HPLC to give compound 13D (230 mg, 45%).

### Step 5: Synthesis of Compound 13

To a solution of compound 13D (120 mg, 0.255 mmol, 1.0 eq) in H₂O/ACN (1 mL/1 mL) was added 3 N NaOH (0.17 mL, 0.509 mmol, 2 eq) and the mixture was stirred at rt for 2 h. To the reaction mixture were slowly added TFA (0.06 mL) and TES (0.06 mL), followed by i-BuB(OH)₂ (52 mg, 0.509 mmol, 2 eq). The resulting mixture was stirred at rt for 1 h, concentrated and purified by prep-HPLC (under acidic conditions) to give an acid form of compound 13 (25 mg).

To a solution of the acid form of compound 13 (25 mg, 0.089 mmol, 1.0 eq) in H₂O/ACN (0.2 mL/0.2 mL) was added 3 N NaOH (0.06 mL, 0.18 mmol, 2 eq). The mixture was stirred at rt for 0.5 h, and purified by prep-HPLC (under neutral conditions) to give compound 13 (17 mg, 55%).

LC-MS: 280 [M+H]⁺; 278 [M−H]⁻
¹H NMR (400 MHz, D₂O) δ8.01 (s, 1H), 7.01-6.99 (d, J=8.4 Hz, 1H), 6.44-6.42 (d, J=8.0 Hz, 1H), 3.75 (s, 3H), 3.51-3.49 (d, J=5.6 Hz, 2H), 2.89-2.85 (m, 1H), 0.63-0.59 (m, 1H), 0.36-0.30 (m, 1H).

### Example 13

### Disodium Salt of 4-(aminomethyl)-2-hydroxy-7-methoxy-3,4-dihydro-1,2-benzoxaborinine-8-carboxylic Acid (Compound 14)

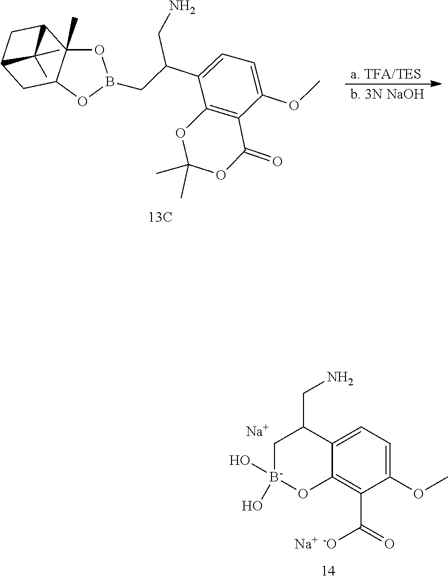

To a solution of compound 13C (180 mg, 0.406 mmol, 1.0 eq) in H₂O/ACN (1 mL/1 mL) was added TFA (0.2 mL) and TES (0.2 mL), followed by i-BuB(OH)₂ (83 mg, 0.812 mmol, 2 eq). The mixture was stirred at rt for 5 h. The reaction mixture was concentrated under reduced pressure. The resulting residue was dissolved in CH₃CN/H₂O and the pH of the solution was adjusted with 3 N NaOH to pH ˜10. The mixture was purified by prep-HPLC (under neutral conditions) to give compound 14 (30 mg, 29%).

### LC-MS: 252 [M+H]⁺

¹H NMR (400 MHz, D₂O) δ6.95-6.93 (d, J=8.4 Hz, 1H), 6.41-6.39 (d, J=8.8 Hz, 1H), 3.71 (s, 3H), 3.15-3.07 (m, 3H), 0.62-0.60 (m, 1H), 0.45-0.41 (m, 1H).

### Example 14

### Disodium Salt of 4-(Azidomethyl)-2-hydroxy-7-methoxy-3,4-dihydro-1,2-benzoxaborinine-8-carboxylic Acid (Compound 15)

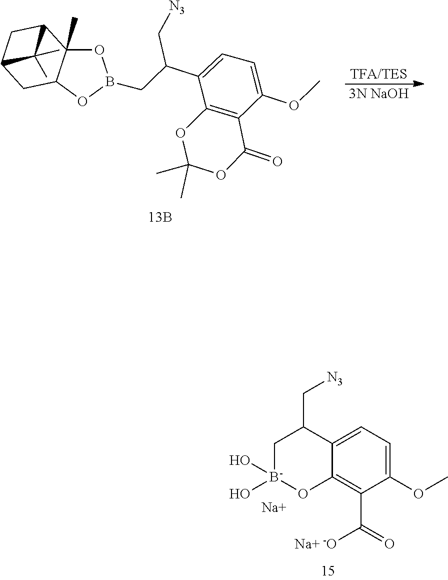

To a solution of compound 13B (200 mg, 0.255 mmol, 1.0 eq) in H₂O/ACN (0.5 mL/0.5 mL) was added TFA (0.06 mL) and TES (0.06 mL), followed by i-BuB(OH)₂ (87 mg, 0.853 mmol, 2 eq). The mixture was stirred at 27° C. for 8 h. The reaction mixture was subsequently concentrated at rt and 3 N NaOH (0.28 mL, 0.84 mmol, 2 eq) was added to the crude. The mixture was stirred at rt overnight, concentrated, and purified by prep-HPLC (under neutral conditions) to give compound 15 (39 mg, 33%).

LC-MS: 278 [M+H]⁺, 555 [2M+H]⁺, and 535 [2M−H]⁻
¹H NMR (400 MHz, D₂O) δ7.03-7.01 (d, J=8.0 Hz, 1H), 6.45-6.42 (d, J=8.4 Hz, 1H), 3.72(s, 3H), 3.58-3.52 (m, 2H), 2.92 (m, 1H), 0.73-0.71 (m, 1H), 0.48 (m, 1H).

### Example 15

### Disodium Salt of 2-hydroxy-4-[[4-(hydroxymethyl)triazol-1-yl]methyl]-7-methoxy-3,4-dihydro-1,2-benzoxaborinine-8-carboxylic Acid (Compound 16)

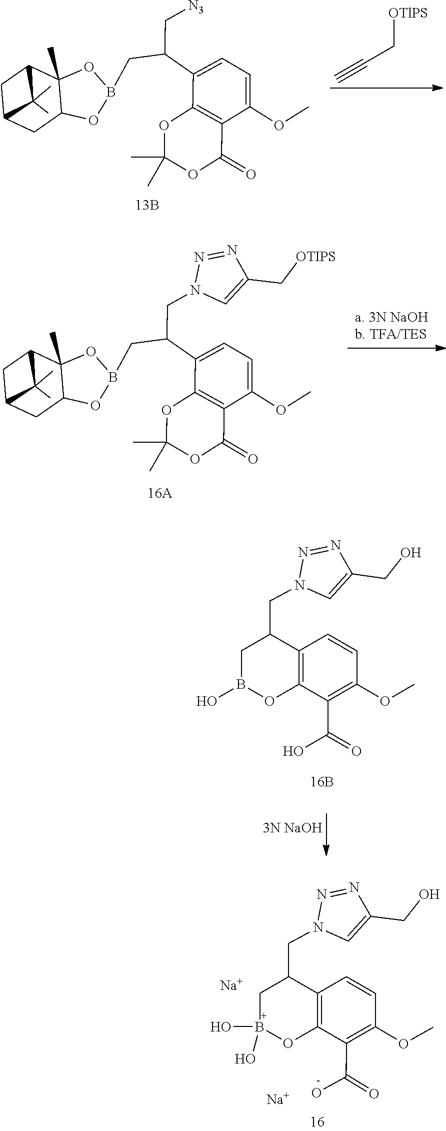

### Step 1: Synthesis of Compound 16A

To a solution of compound 13B (140 mg, 0.30 mmol, 1.0 eq) in 1,2-dichlorobenzene (2 mL) was added triisopropyl(prop-2-ynoxy)silane (127 mg, 0.60 mmol, 2.0 eq). The reaction was stirred at 120° C. overnight under nitrogen atmosphere. The resulting mixture was concentrated under vacuum. The residue was purified by prep-TLC (PE/EA=1:1) to give compound 16A (80 mg, 51%).

Step 2: Synthesis of compound 16

To a solution of compound 16A (80 mg, 0.12 mmol, 1.0 eq) in H₂O/ACN (1 mL/1 mL) was added 3 N NaOH (0.12 mL, 0.36 mmol, 3 eq). The mixture was stirred at rt overnight, followed by a slow addition of TFA (1 mL), TES (1 mL), and i-BuB(OH)₂ (23 mg, 0.235 mmol, 2 eq). The reaction was stirred at rt for an additional 1 h, concentrated, and purified by prep-HPLC (under acidic conditions) to give compound 16B (27 mg) in acid form. To a solution of the compound 16B acid (27 mg, 0.081 mmol, 1.0 eq) in H₂O/ACN (0.2 mL/0.2 mL) was added 3 N NaOH (0.06 mL, 0.18 mmol, 2 eq). The mixture was stirred at rt for 30 min, then triturated with H₂O/acetone (1:20) to give compound 16 (17 mg, 53%).

### LC-MS: 334 [M+H]⁺

¹H NMR (400 MHz, CD₃OD) δ7.58 (s, 1H), 6.40-6.38 (d, J=8.4 Hz, 1H), 6.15-6.13 (d, J=8.4 Hz, 1H), 4.64-4.61 (m, 3H), 4.46 (m, 1H), 3.69 (s, 3H), 3.22-3.19 (m, 1H), 0.51-0.49 (d, J=5.6 Hz, 2H).

### Example 16

### Disodium Salt of 4-(acetamidomethyl)-2-hydroxy-7-methoxy-3,4-dihydro-1,2-benzoxaborinine-8-carboxylic Acid (Compound 17)

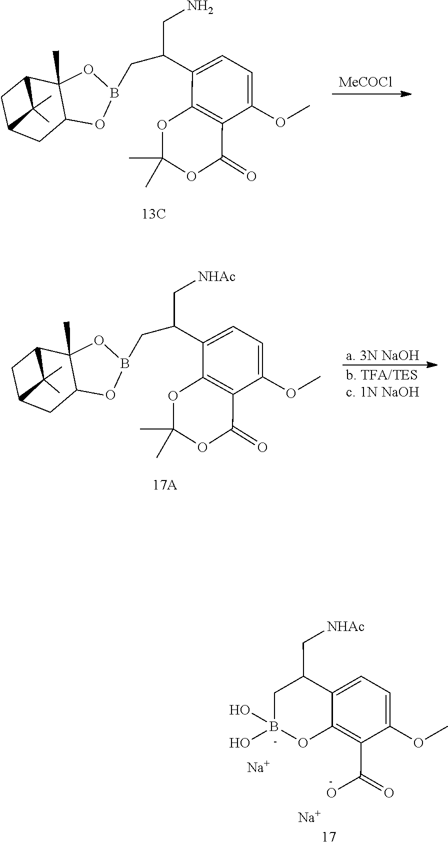

### Step 1: Synthesis of Compound 17A

To a mixture of compound 13C (150 mg, 0.34 mmol, 1.0 eq) in DCM (5 mL) was added acetyl chloride (52 mg, 0.68 mmol, 2.0 eq) and triethylamine (0.14 mL, 1.02 mmol, 3.0 eq) under N₂. The reaction was stirred at rt for 1 h. The resulting mixture was diluted with DCM and washed with water to give crude compound 17A (160 mg, 92%), which was used for next step without further purification.

### Step 2: Synthesis of Compound 17

To a solution of compound 17A (150 mg, 0.31 mmol, 1.0 eq) in H₂O/ACN (1.5 mL/1.5 mL) was added 3 N NaOH (0.31 mL, 0.93 mmol, 3 eq). The mixture was stirred at rt for 3 h, then TFA (1.5 mL) and TES (1.5 mL) were slowly added, followed by i-BuB(OH)₂ (62 mg, 0.62 mmol, 2 eq). The resulting mixture was stirred at rt for 1 h, then purified by prep-HPLC (under acidic conditions) to give an acid form of 17. To a solution of the acid form of 17 (62 mg, 0.21 mmol, 1.0 eq) in H₂O/ACN (0.2 mL/0.2 mL) was added 1 N NaOH (0.41 mL, 0.41 mmol, 2 eq). The mixture was stirred at rt for 30 min, and lyophilized. The solid was triturated with a solution of water and acetone (1:20), and dried to give compound 17 (40 mg, 22%).

LC-MS: 294 [M+H]⁺, 292 [M−H]⁻
¹H NMR (400 MHz, CD₃OD) δ6.83-6.82 (d, J=8.0 Hz, 1H), 6.26-6.24 (d, J=8.4 Hz, 1H), 3.77 (s, 3H), 3.39-3.38 (m, 2H), 2.88-2.85 (m, 1H), 1.88-1.86 (m, 3H), 0.63-0.58 (dd, J=6.8 Hz and 6.0 Hz, 1H), 0.49-0.44 (dd, J=5.2 Hz and 5.2 Hz, 1H).

### Example 17

### Potentiation of Aztreonam

The potency and spectrum of β-lactamase inhibitors (BLIs) was determined by assessing their aztreonam potentiation activity in a dose titration potentiation assay using strains of various bacteria that are resistant to aztreonam due to expression of various β-lactamases. Aztreonam is a monobactam antibiotic and is hydrolyzed by the majority of β-lactamases that belong to class A or C (but not class B or D). The potentiation effect was observed as the ability of BLI compounds to inhibit growth in the presence of sub-inhibitory concentration of aztreonam. MICs of test strains varied from 64 μg/mL to >128 μg/mL. Aztreonam was present in the test medium at 4 μg/mL. Compounds were tested at concentrations up to 20 μg/mL. In this assay, potency of compounds was reported as the minimum concentration of BLI required to inhibit growth of bacteria in the presence of 4 μg/mL of aztreonam (MPC_{@4}). Table 1 summarizes the BLI potency of aztreonam potentiation (MPC_{@4}) for various strains overexpressing class A (ESBL and KPC), and class C β-lactamases. Aztreonam MIC for each strain is also shown.

TABLE 1
Activity of BLIs to potentiate aztreonam against
strains expressing class A and class C enzymes.
Table 1.

| | Aztreonam MIC (μg/mL) | | | | | | | |
|---|---|---|---|---|---|---|---|---|
| | >128 | >128 | >128 | 64 | 128 | >128 | | >128 |
| | AZT | AZT | AZT | AZT | AZT | AZT | 64 | AZT |
| | MPC4 | MPC4 | MPC4 | MPC4 | MPC4 | MPC4 | AZT | MPC4 |
| | CTX-M-14 | CTX-M-15 | SHV-5 | SHV-12 | TEM-10 | KPC-2 | MPC4 | CMY-6 |
| Compound | KP1005 | KP1009 | ec308 | KP1010 | ec302 | KP1004 | ECL1002 | EC1010 |
| 1 | Z | Y | Y | X | Y | X | Y | X |
| 2 | Z | Z | X | X | Y | Y | Y | Y |
| 3 | X | X | X | X | X | X | X | X |
| 4 | X | X | X | X | X | X | X | X |
| 5 | X | X | X | X | X | X | X | X |
| 6 | X | X | X | X | X | X | X | X |
| 7 | X | X | X | X | X | X | X | X |
| 8 | X | X | X | X | X | X | X | X |
| 9 | X | X | X | X | X | X | X | X |
| 10 | Z | Z | X | X | X | X | Y | Y |
| 11 | X | X | X | X | X | X | X | X |
| 12 | Z | Z | X | X | Z | X | Y | Y |
| 13 | X | X | X | X | X | X | X | X |
| 14 | Y | Y | Y | Y | Y | X | Y | Y |
| 15 | X | X | X | X | X | X | X | X |
| 16 | X | X | X | X | X | X | X | X |
| 17 | Y | X | X | X | X | X | X | X |
| Tazobactam | Y | Y | Y | X | X | Z | Z | Y |
| Clavulanic | X | X | X | X | X | Z | Z | Z |
| Acid | | | | | | | | |

X = MPC_{@4} ≤ 5 μg/mL
Y = 5 μg/mL < MPC_{@4} ≤ 20 μg/mL
Z = MPC_{@4} > 20 μg/mL

### Example 18

### Potentiation of Tigemonam

Selected β-lactamase inhibitors were also tested for their ability to potentiate the monobactam tigemonam. The potentiation effect was observed as the ability of BLI compounds to inhibit growth in the presence of sub-inhibitory concentration of tigemonam. MICs of test strains varied from 16 μg/mL to >64 μg/mL. Tigemonam was present in the test medium at 4 μg/mL. Compounds were tested at concentrations up to 20 μg/mL. In this assay, potency of compounds was reported as the minimum concentration of BLI required to inhibit growth of bacteria in the presence of 4 μg/mL of tigemonam (MPC@4). Table 2 summarizes the BLI potency of tigemonam potentiation (MPC@4) for various strains overexpressing class A (ESBL) and class C β-lactamases. Tigemonam MIC for each strain is also shown.

TABLE 2
Activity of BLIs to potentiate tigemonam against
strains expressing class A and class C enzymes.
Table 2.

| | Tigemonam MIC (μg/mL) | | | | | | |
|---|---|---|---|---|---|---|---|
| | >64 | >64 | >64 | >64 | >64 | | 16 |
| | TIG | TIG | TIG | TIG | TIG | 32 | TIG |
| | MPC₄ | MPC₄ | MPC₄ | MPC₄ | MPC₄ | TIG | MPC₄ |
| | CTX-M-14 | CTX-M-15 | SHV-5 | SHV-12 | TEM-10 | MPC₄ | CMY-6 |
| Compound | KP1005 | KP1009 | ec308 | KP1010 | ec302 | ECL1002 | EC1010 |
| 1 | Z | Z | Z | Z | Z | X | X |
| 2 | Z | Z | Y | Y | Z | X | X |
| 3 | X | X | X | X | X | X | X |
| 4 | Y | X | X | X | Y | X | X |
| 5 | X | X | X | X | X | X | X |
| 6 | X | X | X | X | X | X | X |
| 7 | Y | X | X | X | X | X | X |
| 8 | X | X | X | X | X | X | X |
| 9 | Y | X | X | X | X | X | X |
| 10 | Z | Z | X | X | Y | X | X |
| 11 | X | X | X | X | X | X | X |
| 12 | Z | Z | Z | Y | Z | X | X |
| 13 | Y | X | X | X | Y | X | X |
| 14 | Z | Z | Z | Y | Z | X | X |
| 15 | X | X | X | X | X | X | X |
| 16 | Y | X | X | X | Y | X | X |
| 17 | Y | X | X | X | Y | X | X |
| Tazobactam | Y | Y | X | X | X | Y | X |
| Clavulanic | X | X | X | X | X | Z | Z |
| Acid | | | | | | | |

X = MPC_{@4} ≤ 5 μg/mL
Y = 5 μg/mL < MPC_{@4} ≤ 20 μg/mL
Z = MPC_{@4} > 20 μg/mL

### Example 19

### Potentiation of Biapenem

β-lactamase inhibitors were also tested for their ability to potentiate the carbapenem biapenem against strains producing class A (KPC), class D (OXA-48), and class B (metallo β-lactamases, NDM-1 and VIM-1) carbapenemases. The potentiation effect was observed as the ability of BLI compounds to inhibit growth in the presence of a sub-inhibitory concentration of biapenem. Biapenem MIC of test strains were 16-32 μg/mL. Biapenem was present in the test medium at 1 μg/mL. Compounds were tested at concentrations up to 20 μg/mL. In this assay, potency of compounds was reported as the minimum concentration of BLI required to inhibit growth of bacteria in the presence of 1 μg/mL of biapenem (MPC@1). Table 3 summarizes the BLI potency of biapenem potentiation (MPC@1) for four strains overexpressing class A (KPC), class D (OXA-48), and class B (NDM-1 and VIM-1) carbapenemases. Biapenem MIC for each strain is also shown.

TABLE 3
Activity of BLIs to potentiate biapenem against
strains expressing class A (KPC), class D (OXA-48),
and class B (NDM-1 and VIM-1) cabapenemases.
Table 3.

| | Biapenem MIC (μg/mL) | | | |
|---|---|---|---|---|
| | 32 | 16 | 16 | 16 |
| | BPM MPC₁ | BPM MPC₁ | BPM MPC₁ | BPM MPC₁ |
| | KP1004 | OXA-48 | KP1081 | KP1054 |
| Compound | KPC-2 | KP1086 | NDM-1 | VIM-1 |
| 1 | X | X | X | Z |
| 2 | X | X | Z | Z |
| 3 | X | X | X | Y |
| 4 | X | X | X | Y |
| 5 | X | X | Y | Z |
| 6 | X | X | X | Y |
| 7 | X | X | X | Y |
| 8 | X | X | Y | Z |
| 9 | X | X | X | Z |
| 10 | X | X | Z | Z |
| 11 | X | X | X | Y |
| 12 | X | X | Y | Z |
| 13 | X | X | X | Y |
| 14 | X | Y | X | Z |
| 15 | X | X | X | Y |
| 16 | X | Y | Y | Y |
| 17 | X | Y | Y | Y |
| Tazobactam | Z | Y | Z | Z |
| Clavulanic | Y | Z | Z | Z |
| Acid | | | | |

X = MPC_{@1} ≤ 5 μg/mL
Y = 5 μg/mL < MPC_{@1} ≤ 20 μg/mL
Z = MPC_{@1} > 20 μg/mL

### Example 20

### Potentiation of Meropenem

β-lactamase inhibitors were also tested for their ability to potentiate the carbapenem meropenem against strains of Acinetobacter baumannii producing class D (OXA-23 and OXA-72) carbapenemases. The potentiation effect was observed as the ability of BLI compounds to inhibit growth in the presence of a sub-inhibitory concentration of meropenem. Meropenem MIC of test strains were 32 to >64 μg/mL. Meropenem was present in the test medium at 8 μg/mL. Compounds were tested at concentrations up to 20 μg/mL. In this assay, potency of compounds was reported as the minimum concentration of BLI required to inhibit growth of bacteria in the presence of 8 μg/mL of meropenem (MPC@8). Table 4 summarizes the BLI potency of meropenem potentiation (MPC@s) for two strains overexpressing OXA-72 and OXA-23 carbapenemases. Meropenem MIC for each strain is also shown.

TABLE 4
Activity of BLIs to potentiate meropenem
against strains expressing class D carbapenemases
from Acinetobacter baumannii
Table 4.

Meropenem MIC (μg/mL)

| | >64 | 32 |
|---|---|---|
| | MPM MPC₈ | MPM MPC₈ |
| | AB1053 | AB1054 |
| Compound | OXA-72 | OXA-23 |
| 1 | Z | Z |
| 2 | Y | Y |
| 3 | X | X |
| 4 | X | X |
| 5 | X | X |
| 6 | X | X |
| 7 | Y | Y |
| 8 | X | X |
| 9 | X | Y |
| 10 | Z | Z |
| 11 | X | X |
| 12 | X | Y |
| 13 | X | X |
| 14 | Y | Z |
| 15 | X | X |
| 16 | X | Y |
| 17 | X | Y |

X = MPC_{@1} ≤ 5 μg/mL
Y = 5 μg/mL < MPC_{@1} ≤ 20 μg/mL
Z = MPC_{@1} > 20 μg/mL

What is claimed is:

1. A method of treating a bacterial infection, comprising administering to a subject in need thereof, one or more compounds having the structure of Formula (I) or Formula (II): 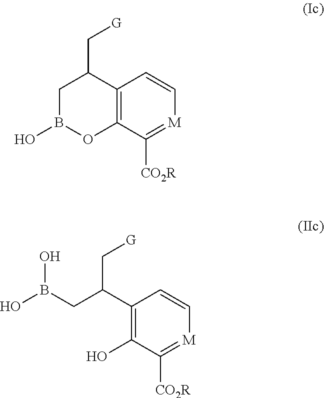
or a pharmaceutically acceptable salt thereof, wherein:
G is selected from the group consisting of —OR¹—C(O)R¹, —C(O)(CH₂)₀₋₃SR¹, —C(O)(CH₂)₁₋₃R¹, —C(O)OR¹, —C(O)NR¹R², —C(O)NR¹OR², —N₃, —NR¹R², —NR¹C(O)R², —NR¹C(O)NR²R³, —NR¹C(O)OR², —NR¹S(O)²R², —NR¹S(O)₂NR²R³, —C(═NR¹)R², —C(═NR¹)NR²R³, —NR¹CR²(═NR³), —NR¹C(═NR²)NR³R⁴, —S(O)₂R¹, optionally substituted C₁₋₁₀ alkyl, optionally substituted C₂₋₁₀ alkenyl, optionally substituted C₂₋₁₀ alkynyl, optionally substituted C₃₋₇ carbocyclyl, optionally substituted 5-10 membered heterocyclyl, optionally substituted C₆₋₁₀ aryl, optionally substituted 5-10 membered heteroaryl, optionally substituted C₃₋₇ carbocyclyl-C₁₋₆ alkyl, optionally substituted 5-10 membered heterocyclyl-C₁₋₆ alkyl, optionally substituted C₆₋₁₀ aryl-C₁₋₆ alkyl, and optionally substituted 5-10 membered heteroaryl-C₁₋₆ alkyl;
R¹, R², R³, and R⁴ are each independently selected from the group consisting of —H, optionally substituted C₁₋₄ alkyl, optionally substituted C₃₋₇ carbocyclyl, optionally substituted 4-10 membered heterocyclyl, optionally substituted C₆₋₁₀ aryl, optionally substituted C₆₋₁₀ aryl-C₁₋₆ alkyl, and optionally substituted 5-10 membered heteroaryl;
J is selected from the group consisting of CR⁵ and N;
L is selected from the group consisting of CR⁶ and N;
M is selected from the group consisting of CR⁷ and N;
R⁵, R⁶, and R⁷ are each independently selected from the group consisting of —H, —OR⁸, halogen, —CF₃, optionally substituted C₂-C₆ alkenyl, optionally substituted C₂-C₆ alkynyl, optionally substituted C₁-C₆ heteroalkyl, optionally substituted C₃-C₇ carbocyclyl, optionally substituted 5-10 membered heterocyclyl, optionally substituted C₆₋₁₀ aryl, optionally substituted 5-10 membered heteroaryl, cyano, C₁-C₆ alkoxy(C₁-C₆)alkyl, aryloxy, and sulfhydryl (mercapto);
R⁸ is selected from the group consisting of hydrogen, optionally substituted C₁₋₄ alkyl, optionally substituted C₃₋₇ carbocyclyl, optionally substituted 4-10 membered heterocyclyl, optionally substituted C₆₋₁₀ aryl, and optionally substituted 5-10 membered heteroaryl;
R is selected from the group consisting of —H, —C₁₋₉ alkyl, —CR⁹R¹⁰OC(O)C₁₋₉alkyl, —CR⁹R¹⁰OC(O)OC₁₋₉alkyl, —CR⁹R¹⁰OC(O)C₆₋₁₀aryl, —CR⁹R¹⁰OC(O)OC₆₋₁₀aryl, 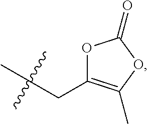 [SMILES: *Cc1oc(=O)oc1C]
—CR⁹R¹⁰OC(O)C₃₋₇carbocyclyl, —CR⁹R¹⁰OC(O)OC₃₋₇carbocyclyl, —CR⁹R¹⁰OC(O)(5-10 membered heterocyclyl), and —CR⁹R¹⁰OC(O)O(5-10 membered heterocyclyl); and
R⁹ and R¹⁰ are independently selected from the group consisting of —H, optionally substituted C₁₋₄ alkyl, optionally substituted C₃₋₇ carbocyclyl, optionally substituted 5-10 membered heterocyclyl, optionally substituted C₆₋₁₀ aryl, and optionally substituted 5-10 membered heteroaryl.

2. The method of claim 1, wherein the one or more compounds have the structure of Formula (Ic) or Formula (IIc): 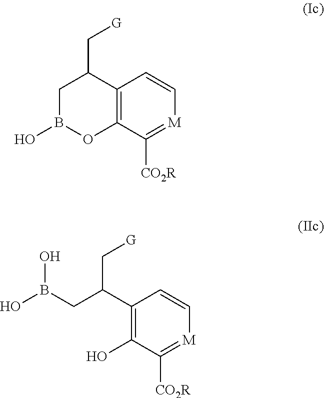
or a pharmaceutically acceptable salt thereof, wherein:
G is selected from the group consisting of —OH, —OMe, —OBn, —CH₂OH, N₃, NH₂, —NHC(═O)H, —NHC(═O)CH₃, and 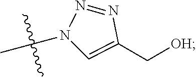 [SMILES: *n1cc(CO)nn1]
R⁷ is selected from the group consisting of —H, —OR⁸, and halogen;
R⁸ is C₁₋₄ alkyl; and
R is —H.

3. The method of claim 1, wherein G is selected from the group consisting of —OH and —OBn.

4. The method of claim 1, wherein the one or more compounds have a structure selected from the group consisting of: 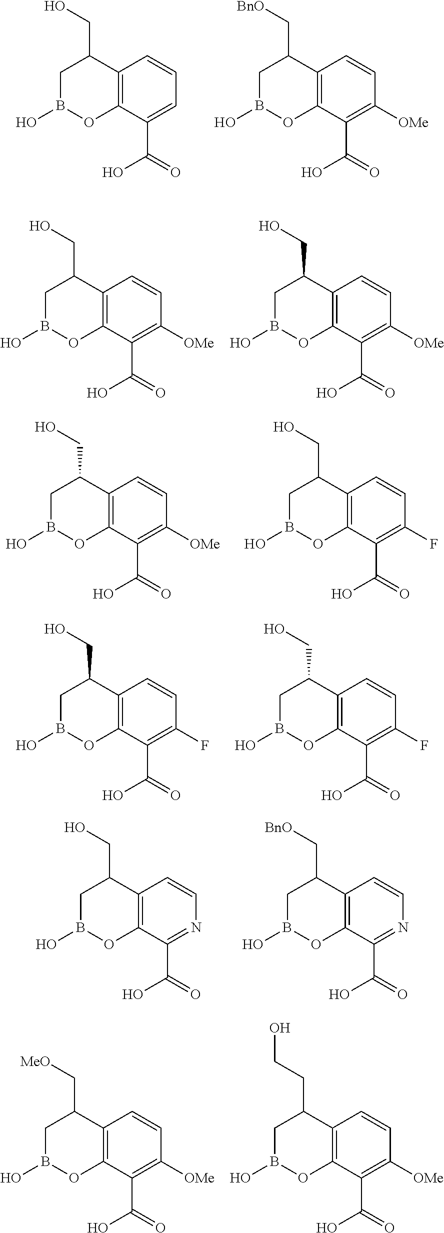 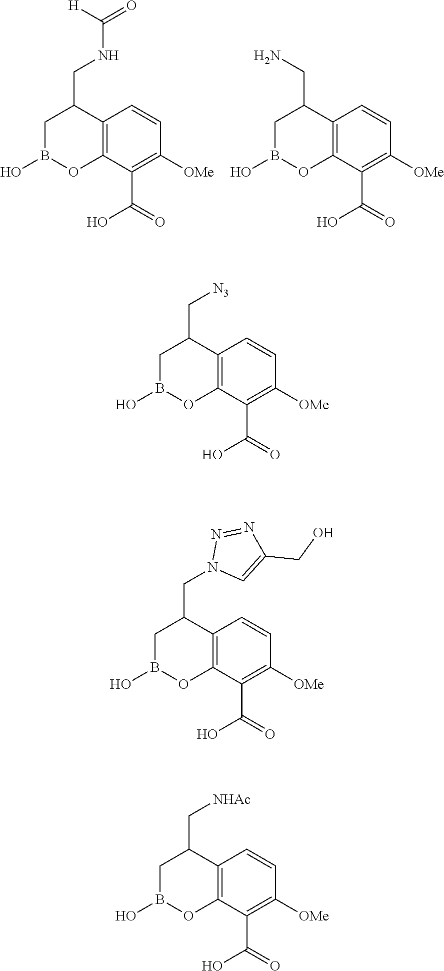 [SMILES: COc1ccc2c(c1C(=O)O)OB(O)CC2CNC=O ; COc1ccc2c(c1C(=O)O)OB(O)CC2CN ; COc1ccc2c(c1C(=O)O)OB(O)CC2CN=[N+]=[N-] ; COc1ccc2c(c1C(=O)O)OB(O)CC2Cn1cc(CO)nn1 ; COc1ccc2c(c1C(=O)O)OB(O)CC2CNC(C)=O]
and pharmaceutically acceptable salts thereof.

5. The method of claim 1, comprising administering to a subject in need thereof, a compound having the structure of: 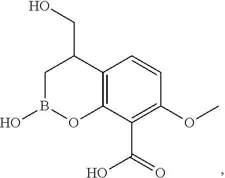 [SMILES: COc1ccc2c(c1C(=O)O)OB(O)CC2CO]
or a pharmaceutically acceptable salt thereof.

6. The method of claim 1, comprising administering to a subject in need thereof, a compound having the structure of: 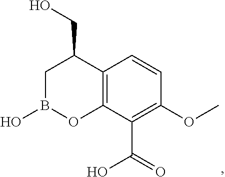 [SMILES: COc1ccc2c(c1C(=O)O)OB(O)C[C@H]2CO]
or a pharmaceutically acceptable salt thereof.

7. The method of claim 1, comprising administering to a subject in need thereof, a compound having the structure of: 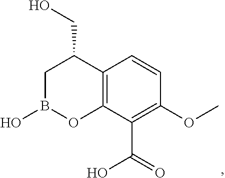 [SMILES: COc1ccc2c(c1C(=O)O)OB(O)C[C@@H]2CO]
or a pharmaceutically acceptable salt thereof.

8. The method of claim 1, comprising administering to a subject in need thereof, a compound having the structure of: 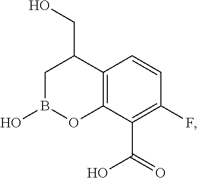 [SMILES: O=C(O)c1c(F)ccc2c1OB(O)CC2CO]
or a pharmaceutically acceptable salt thereof.

9. The method of claim 1, comprising administering to a subject in need thereof, a compound having the structure of: 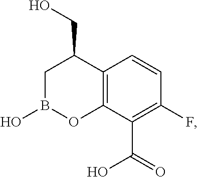 [SMILES: O=C(O)c1c(F)ccc2c1OB(O)C[C@H]2CO]
or a pharmaceutically acceptable salt thereof.

10. The method of claim 1, comprising administering to a subject in need thereof, a compound having the structure of: 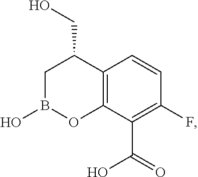 [SMILES: O=C(O)c1c(F)ccc2c1OB(O)C[C@@H]2CO]
or a pharmaceutically acceptable salt thereof.

11. The method of claim 1, further comprising administering to the subject an additional medicament.

12. The method of claim 11, wherein the additional medicament is selected from the group consisting of an antibacterial agent, an antifungal agent, an antiviral agent, an anti-inflammatory agent, and an anti-allergic agent.

13. The method of claim 12, wherein the additional medicament is β-lactam antibacterial agent.

14. The method of claim 13, wherein the β-lactam antibacterial agent is selected from the group consisting of Amoxicillin, Ampicillin (Pivampicillin, Hetacillin, Bacampicillin, Metampicillin, Talampicillin), Epicillin, Carbenicillin (Carindacillin), Ticarcillin, Temocillin, Azlocillin, Piperacillin, Mezlocillin, Mecillinam (Pivmecillinam), Sulbenicillin, Benzylpenicillin (G), Clometocillin, Benzathine benzylpenicillin, Procaine benzylpenicillin, Azidocillin, Penamecillin, Phenoxymethylpenicillin (V), Propicillin, Benzathine phenoxymethylpenicillin, Pheneticillin, Cloxacillin (Dicloxacillin, Flucloxacillin), Oxacillin, Meticillin, Nafcillin, Faropenem, Tomopenem, Razupenem, Cefazolin, Cefacetrile, Cefadroxil, Cefalexin, Cefaloglycin, Cefalonium, Cefaloridine, Cefalotin, Cefapirin, Cefatrizine, Cefazedone, Cefazaflur, Cefradine, Cefroxadine, Ceftezole, Cefaclor, Cefamandole, Cefminox, Cefonicid, Ceforanide, Cefotiam, Cefprozil, Cefbuperazone, Cefuroxime, Cefuzonam, Cefoxitin, Cefotetan, Cefmetazole, Loracarbef, Cefixime, Ceftriaxone, Cefcapene, Cefdaloxime, Cefdinir, Cefditoren, Cefetamet, Cefmenoxime, Cefodizime, Cefoperazone, Cefotaxime, Cefpimizole, Cefpiramide, Cefpodoxime, Cefsulodin, Cefteram, Ceftibuten, Ceftiolene, Ceftizoxime, Flomoxef, Latamoxef, Cefepime, Cefozopran, Cefpirome, Cefquinome, Ceftobiprole, Ceftaroline, CXA-101, RWJ-54428, MC-04,546, ME1036, Ceftiofur, Cefquinome, Cefovecin, RWJ-442831, RWJ-333441, and RWJ-333442.

15. The method of claim 13, wherein the β-lactam antibacterial agent is selected from the group consisting of Ceftazidime, Biapenem, Doripenem, Ertapenem, Imipenem, Meropenem, Tebipenem, Tebipenem pivoxil, Apapenem, and Panipenem.

16. The method of claim 13, wherein the β-lactam antibacterial agent is selected from the group consisting of Aztreonam, Tigemonam, BAL30072, SYN 2416, and Carumonam.

17. The method of claim 1, wherein the subject is a mammal.

18. The method of claim 17, wherein the mammal is a human.

19. The method of claim 1, wherein the infection comprises a bacteria selected from the group consisting of Pseudomonas acidovorans, Pseudomonas alcaligenes, Pseudomonas putida, Burkholderia cepacia, Aeromonas hydrophilia, Francisella tularensis, Morganella morganii, Proteus mirabilis, Proteus vulgaris, Providencia alcalifaciens, Providencia rettgeri, Providencia stuartii, Acinetobacter baumannii, Bordetella pertussis, Bordetella para pertussis, Bordetella bronchiseptica, Haemophilus ducreyi, Pasteurella multocida, Pasteurella haemolytica, Branhamella catarrhalis, Borrelia burgdorferi, Kingella, Gardnerella vaginalis, Bacteroides distasonis, Bacteroides 3452A homology group, Clostridium difficile, Mycobacterium tuberculosis, Mycobacterium avium, Mycobacterium intracellulare, Mycobacterium leprae, Corynebacterium diphtheriae, Corynebacterium ulcerans, Streptococcus pneumoniae, Streptococcus agalactiae, Streptococcus pyogenes, Enterococcus faecalis, Enterococcus faecium, Staphylococcus aureus, Staphylococcus epidermidis, Staphylococcus saprophyticus, Staphylococcus intermedius, Staphylococcus hyicus subsp. hyicus, Staphylococcus haemolyticus, Staphylococcus hominis, and Staphylococcus saccharolyticus.

20. The method of claim 1, wherein the infection comprises a bacteria selected from the group consisting of Pseudomonas aeruginosa, Pseudomonas fluorescens, Stenotrophomonas maltophilia, Escherichia coli, Citrobacter freundii, Salmonella typhimurium, Salmonella typhi, Salmonella paratyphi, Salmonella enteritidis, Shigella dysenteriae, Shigella flexneri, Shigella sonnei, Enterobacter cloacae, Enterobacter aerogenes, Klebsiella pneumoniae, Klebsiella oxytoca, Serratia marcescens, Acinetobacter calcoaceticus, Acinetobacter haemolyticus, Yersinia enterocolitica, Yersinia pestis, Yersinia pseudotuberculosis, Yersinia intermedia, Haemophilus influenzae, Haemophilus parainfluenzae, Haemophilus haemolyticus, Haemophilus parahaemolyticus, Helicobacter pylori, Campylobacter fetus, Campylobacter jejuni, Campylobacter coli, Vibrio cholerae, Vibrio parahaemolyticus, Legionella pneumophila, Listeria monocytogenes, Neisseria gonorrhoeae, Neisseria meningitidis, Moraxella, Bacteroides fragilis, Bacteroides vulgatus, Bacteroides ovalus, Bacteroides thetaiotaomicron, Bacteroides uniformis, Bacteroides eggerthii, and Bacteroides splanchnicus.
